(19) United States
(12) Patent Application Publication
(10) Pub. No.: US 2025/0215059 A1
(43) Pub. Date: Jul. 3, 2025

(54) METHODS AND COMPOSITIONS FOR DISRUPTING TAU AGGREGATES USING POLYSERINE REPEAT SEQUENCES TARGETING EXOGENOUS PROTEINS

(71) Applicant: The Regents of the University of Colorado, a body corporate, Denver, CO (US)

(72) Inventors: Meaghan Van Alstyne, Boulder, CO (US); Roy Parker, Boulder, CO (US); Evan Lester, Denver, CO (US)

(21) Appl. No.: 18/847,671

(22) PCT Filed: Mar. 16, 2023
(86) PCT No.: PCT/US23/64535

§ 371 Date: Sep. 16, 2024

Related U.S. Application Data
(60) Provisional application No. 63/320,288, filed on Mar. 16, 2022.

(51) Int. Cl. C07K 14/47; A61K 38/00; C07K 14/705; C12N 9/14

(52) U.S. Cl. CPC C07K 14/47; C07K 14/705; C12N 9/14; C12Y 306/04006; A61K 38/00; C07K 2319/00

(57) ABSTRACT

The present invention includes novel methods and compositions for targeting tau aggregates, or components thereof. In one specific embodiment, the invention include the novel use of polyserine repeat sequences, and/or serine-rich sequences, which may be coupled with a heterologous peptide, to target tau aggregates, or components thereof, and thereby localize the heterologous peptide to the tau aggregate.

### CROSS-REFERENCE TO RELATED APPLICATIONS

This International PCT Application claims the benefit of and priority to U.S. Provisional Application No. 63/320,288, filed Mar. 16, 2022. The entire specification, claims, and figures of the above-referenced application is hereby incorporated, in its entirety by reference.

### STATEMENT OF GOVERNMENT INTEREST

This invention was made with government support under grant number F30AG063468 awarded by the National Institutes of Health. The government has certain rights in the invention.

### TECHNICAL FIELD

The present invention is related to systems, methods and compositions for generating novel fusion peptides configured to target tau aggregates, or components thereof.

### BACKGROUND

Tau aggregates are insoluble accumulations of tau (MAPT) and other associated components, such as proteins and RNAs that are found in the brains of patients with neurodegenerative tauopathies. There are over 20 different tauopathies including Alzheimer's Disease (AD), frontotemporal dementia (FTD-tau), and Chronic Traumatic Encephalopathy (CTE). Higher order tau assemblies have been shown to be toxic to cells, with smaller soluble aggregates thought to exhibit greater toxicity than large fibrils. What the mechanism of tau aggregate toxicity is and whether this toxicity mediates neurodegeneration in tauopathies are unknown. One possible contribution to the toxicity of tau aggregates is a model where tau aggregates sequester essential RNAs and RNA binding proteins leading to a loss of function and disrupted RNA biogenesis. In patient post-mortem samples, splicing related RNA binding proteins such as SRRM2, U1-70K, and SFPQ have been observed to be enriched in cytoplasmic tau aggregates. Moreover, in both cell line and mouse models of tauopathies tau aggregates can form in nuclear speckles, which are membrane-less organelles containing nascent transcripts and splicing machinery. Some components of splicing speckles can leave the nucleus and abnormally co-localize with cytoplasmic tau aggregates.

As accumulation of misfolded disease-linked proteins is a shared feature across many neurodegenerative diseases, there has been significant investigation into molecular chaperones and disaggregases—proteins which can resolve misfolded toxic species and provide opportunity for refolding or proteolysis. For example, the yeast AAA+ ATPase Hsp104—which is not present in metazoa—has been re-engineered to utilize its potent disaggregase activity against several disease-linked protein aggregates including AB, asynuclein, FUS and TDP-43. Additionally, nuclear import receptors were shown to have chaperone activity that mitigates assembly of intrinsically disordered RNA-binding proteins such as FUS and TDP-43—the accumulation of which is a feature of neurodegenerative diseases such as amyotrophic lateral sclerosis and frontotemporal dementia (ALS/FTD).

Underlining the importance of such cellular disassembly mechanisms in disease, mutations in the main human AAA+ATPase VCP have been linked to TDP-43 accumulation in inclusion body myopathy associated with Paget disease of bone, frontotemporal dementia (IBMPFD), and ALS/FTD (10,11). Further, a hypomorphic mutation in VCP has been associated with tau aggregate formation in vacuolar tauopathy and VCP can disassemble tau aggregates in cellular systems (12,13). One key caveat of aiming to disassemble aggregate-prone species, is that with prion-like proteins such as tau, such intervention may shift the balance from larger aggregated forms to smaller, more potent seeding-competent species which can drive more misfolding (13,14). Nonetheless, identifying proteins that possess disaggregase activity towards tau could provide insight into therapeutic strategies and mechanisms to enhance this function—such as through promoting association with toxic tau species—could further enhance effects.

Herein, the present inventors use a tau aggregate seeding model expressing fluorescently tagged versions of tau and the canonical splicing speckle protein, SRRM2, to address these issues. The present invention describes the novel mechanisms whereby SRRM2 and its binding partner PNN accumulate in cytoplasmic tau aggregates after its translocation to the cytoplasm. Additionally, the present inventors mapped the tau aggregate interacting region of SRRM2 and PNN to polyserine repeats in the c-terminus of the protein and show that polyserine repeats are sufficient to enrich fusion proteins in both nuclear and cytoplasmic tau aggregates. Together this novel inventive feature identifies mechanisms by which nuclear splicing speckle proteins re-localize to the cytoplasm. As demonstrated below, polyserine motifs can be used to target fusion proteins to aggregating tau and thereby limit tau aggregation. Specifically, Applicants identified that TNPO3, VCP, and most potently UBXD8/FAF2 suppress tau aggregation in a manner augmented by polyserine-based targeting. Mechanistically, UBXD8/FAF2 requires domains facilitating ubiquitin recognition, VCP recruitment and membrane localization for its activity in limiting tau aggregation. Thus, the invention highlights polyserine as a modular tau targeting motif and provide mechanistic insight into the suppressive function of, in particular UBXD8/FAF2 on aggregation of tau.

### SUMMARY OF THE INVENTION

The present invention includes systems, methods and compositions to target tau aggregates, in in particular the use of polyserine repeat domains to target heterologous proteins to a tau aggregate. To date, there are not known mechanisms of targeting a protein to a tau aggregate. In a preferred embodiment, using polypeptide repeats such as polyserine repeat or serine rich domains as a targeting mechanism allows to the specific delivery of therapeutically or metabolically active heterologous peptides to target the pathogenic, aggregated form of tau but not the physiologic, soluble form of tau. This may allow for targeted modulation of tau toxicity that preserves tau's normal function in healthy cells.

In another aspect, the invention may include the use of a region of the SRRM2 protein (SEQ ID NO. 2), the PNN protein (c-terminal region according to SEQ ID NO. 26, 27), or polypeptide repeats, and preferably polyserine repeats, to generate fusion peptides targeted proteins to tau aggregates that form in the brains of patients with neurodegenerative tauopathies. These polypeptide targeting motifs can be fused to a variety of other proteins to target insoluble tau aggregates for degradation, imaging, and/or preventing the association of endogenous molecules with tau aggregates. Some of the proteins that could be potentially fused to this targeting motif include the following: E3 ubiquitin ligase to degrade tau aggregates, VCP to unwind and separate tau aggregates, transportin 3, nucleases to degrade RNA locally around the tau aggregate, radioligands and fluorescent ligands for imaging applications, kinases/phosphatases/acetyl transferases and the like, to modify post-translational modification status of tau aggregates, VCP adaptors, such as UBXD8/FAF2. These fusion constructs could be engineered into expression vectors and delivered to a target cell, such as through via pharmaceutical compositions, such as adeno-associated virus vectors to animal model systems and patient brains, or other similar pharmaceutical composition delivery mechanisms such as lipid-nanoparticles (LNPs), and/or exosomes and the like. The present inventors further have successfully made a plurality of such constructs incorporating polyserine repeat motifs and peptides, such as TNPO3, VCP, and most potently UBXD8/FAF2, that inhibit decrease tau aggregation in a HEK293 tau biosensor cell line.

Additional aspects of the invention include novel methods and composition to inhibit tau aggregate formation in a cell. In a preferred embodiment, expression of PNN may be downregulated in a cell, and preferably the cell of a human subject, through one or more inhibitory RNA molecules, and in particular a small inhibitory RNA (siRNA) molecules directed to inhibit the expression of PNN, having a sense strand selected from the group consisting of: SEQ ID NO.'s 41-43.

Additional aspects of the invention may include one or more of the preferred embodiments set forth in the claims.

### BRIEF DESCRIPTION OF THE FIGURES

FIGS. 1A-E: Mechanisms of SRRM2 incorporation into cytoplasmic tau aggregates. A) Still frames the formation of a cytoplasmic SRRM2 assembly in the cytoplasm and the growth of a cytoplasmic tau aggregate growing from the surface of the assembly. B) Stills showing the formation of SRRM2+ MIGs that nucleate tau aggregates from the surface, which then merge with one another. C) Stills showing a nuclear implosion event where a cytoplasmic tau aggregate forms, followed by chromatin condensation, and a sudden movement of nuclear SRRM2 into the cytoplasmic tau aggregate. D) Quantification of 75 SRRM2 relocalization events over 48 hours of imaging at a timestep of 10 minutes showing the percentage of total events made up by each of the three mechanisms. E) Images of dividing and non-dividing cells with SRRM2 positive cytoplasmic assemblies. Arrows indicate the location of cytoplasmic assemblies and highlight the lack of tau-YFP enrichment in these structures in the absence of aggregated tau.

FIGS. 2A-G: SRRM2 c-terminus is necessary for localization to tau aggregates. A) Schematic showing the SRRM2-Halo truncation constructs that were expressed by different CRISPR edited HEK293 tau biosensor cell lines. B) A 4-12% Bis-Tris poly acrylamide gel showing halo tagged SRRM2 constructs. Bands are JF646 halo ligand that was covalently linked to SRRM2-Halo fusion proteins by addition to cell culture at 200nM overnight prior to lysis. C) Representative images of SRRM2_FL-Halo, SRRM2_1-Halo, and SRRM2_2-Halo showing loss of colocalization in cytoplasmic tau aggregates with loss of the c-terminus. For images of all constructs, see FIGS. 6A, D, E) Quantification of Halo enrichment in cytoplasmic and nuclear tau aggregates for twenty-five images per condition. Enrichment was defined as the mean Halo intensity within cytoplasmic or nuclear tau aggregates divided by the mean Halo intensity in the cytoplasm and nucleus, respectively. Mean and 95% confidence intervals are shown. F) Representative images of MIGs in SRRM2_1-Halo and SRRM2_2-Halo where there is a significant drop off the in the number of MIGs that form in cells without tau aggregates. G) Quantification of the number of SRRM2+ MIGs that form per nuclei in cells expressing the various SRRM2-Halo truncation constructs. Twenty-five images per condition were quantified using Ilastik and CellProfiler. Mean and 95% confidence intervals are shown.

FIG. 3A: Representative images of SRRM2-Halo truncations. A) Representative images of the nine cell lines analyzed in FIG. 10. All cell lines were incubated with 200 nM JF646 and seeded with tau aggregates for 24 hours prior to fixation and imaging. Loss of Halo colocalization with cytoplasmic tau aggregates can be seen in truncations 2-7 and loss of nuclear speckles can be seen in truncations 6-7.

FIGS. 4A-C: SRRM2 c-terminus is sufficient for localization to tau aggregates. A) Schematic of the Halo tagged C-terminal fragment constructs that were PCR amplified from the SRRM2_FL-Halo cell line and cloned into a pcDNA mammalian expression plasmid. The locations of the serine repeats are shown in yellow. B) Quantification of the enrichment of the various Halo tagged constructs in cytoplasmic tau aggregates. Cells both expressing the halo tagged construct and containing a tau aggregate were identified by hand, and the average Halo intensity within the tau aggregates were normalized by an adjacent region outside of the tau aggregate to calculate enrichment. Five aggregates per condition were quantified, mean and 95% confidence interval is shown, p-values were calculated using an unpaired t-test. C) Representative images of cells expressing the various Halo tagged C-terminal SRRM2 fragments. Cells were labeled with 200 nM JF646 added directly to culture media for 24 hours prior to fixation and imaging.

FIGS. 5A-F: Polyserine is sufficient for localization to tau aggregates. A) Representative images of HEK293 tau biosensor cells expressing Halo tagged 5, 10, 20, 42 serine repeats. Enrichment can be seen in 20 and 42 serine repeats, but not in 5 or 10 repeats. Cells were labeled with 200 nM JF646 for 24 hours prior to fixation and imaging. B) A 4-12% Bis-Tris poly acrylamide gel showing halo tagged polyserine constructs with and without the presence of tau aggregates. Bands are JF646 halo ligand that was covalently linked to polyserine-Halo fusion proteins by addition to cell culture at 200 nM overnight prior to lysis. lug of plasmid was transfected into cells using lipofectamine 3000 24 hours prior to cell lysis. C) Quantification of the enrichment of the Halo tagged polyserine constructs in cytoplasmic tau aggregates. Cells both expressing the halo tagged construct and containing a tau aggregate were identified by hand, and the average Halo intensity within the tau aggregates were normalized by an adjacent region outside of the tau aggregate to calculate enrichment. 5 aggregates per condition were quantified, mean and 95% confidence interval is shown, p-values were calculated using an unpaired t-test. D) comparison of % of tau aggregates colocalized with SRRM2_FL-halo, Frag_2-Halo, and 42-polyserine-halo. 5 images per condition were quantified, mean and 95% confidence interval is shown, p-values were calculated using an unpaired t-test. E) Image of PNN and SETDIA, two polyserine repeat containing proteins that display enrichment in tau aggregates.

FIGS. 6A-D: SRRM2 C-terminus and 42-polyserine can grow tau aggregates from their surface. A) still images taken from movies of HEK293 tau biosensor cells forming tau aggregates in the presence of overexpressed SRRM2 C-terminus-halo. B) Quantification of 50 tau aggregate nucleation events that occurred in cells expressing SRRM2 C-terminus-halo. Shows the proportion of nucleation events that were associated with SRRM2 C-terminal assemblies. C) Still images taken from movies of HEK293 tau biosensor cells forming tau aggregates in the presence of overexpressed 42-polyserine-halo. D) quantification of 50 tau aggregate nucleation events that occurred in cells expressing 42-polyserine-halo. Shows the proportion of nucleation events that were associated with SRRM2 C-terminal assemblies.

FIGS. 7A-F: Knockdown of PNN decreases tau aggregation. PNN-C-Terminus is recruited to tau aggregates. 42 polyserine is recruited to tau aggregates in H4 cells expressing ON4R full length tau. Polyserine containing proteins create assemblies in cells. Knockdown of PNN using siRNAs in HEK293 tau biosensor cells. Tau aggregation was measured two ways using flow cytometry A) integrated fret density and B) % of FRET+cells. C) PNN C-terminus-halo is recruited to tau aggregates in HEK293 tau biosensor cells. D) 42-polyserine is recruited to tau aggregates in H4 cells expressing full length ON4R tau. E, F) both SRRM2 c-terminus-halo and 42-polyserine-halo form cytoplasmic assemblies when overexpressed in HEK293 tau biosensor cells.

FIGS. 8A-J: Polyserine targets a range of proteins to tau aggregates. (A) Schematic of constructs used for transient transfection in B. (B) Immunostaining of DAPI, Tau-YFP and JF646 labeled Halo ligand in HEK293T tau biosensor cells transiently transfected with Halo, 42 polyserine (PS), and SRRM2 C-terminal constructs. (C) Schematic of constructs used for transient transfection in D. (D) Immunostaining of DAPI, Tau-YFP and JF646 labeled Halo ligand in HEK293T tau biosensor cells transiently transfected with HSC70 fused to Halo, 42 polyserine (PS), or the SRRM2 C-terminal fragment at the N-terminus. (E) Schematic of constructs used for transient transfection in F. (F) Immunostaining of DAPI, Tau-YFP and JF646 labeled Halo ligand in HEK293T tau biosensor cells transiently transfected with LC3B fused to Halo, 42 polyserine (PS), or the SRRM2 C-terminal fragment at the C-terminus. (G) Schematic of constructs used for transient transfection in H. (H) Immunostaining of DAPI, Tau-YFP and JF646 labeled Halo ligand in HEK293T tau biosensor cells transiently transfected with TNPO1 fused to Halo, 42 polyserine (PS), or the SRRM2 C-terminal fragment at the N-terminus. (I) Schematic of constructs used for transient transfection in J. (J) Immunostaining of DAPI, Tau-YFP and JF646 labeled Halo ligand in HEK293T tau biosensor cells transiently transfected with TNPO1 fused to Halo, 42 polyserine (PS), or the SRRM2 C-terminal fragment at the N-terminus.

FIGS. 9A-H. Polyserine-based targeting can reduce tau aggregation in cells. (A) Representative gating to sort cell population in FACS of HEK293T tau biosensor cells. (B) Representative gating to sort single cell population in FACS of HEK293T tau biosensor cells. (C-D) Representative gating of sorting based on Halo expression in HEK293T tau biosensor cells mock transfected (C) or expression Halo (D). (E-F) Representative gating of sorting for FRET positivity of HEK293T tau biosensor cells mock seeded (E) or seeded with tau brain homogenate (F). (G) Percentage of FRET positive cells normalized to Halo transfected control expressing Halo, 42 polyserine (PS) Halo, or SRRM2 C-terminus Halo alone or fused to either TNPO3 or VCP. (H) Integrated FRET density normalized to Halo transfected control expressing Halo, 42 polyserine (PS) Halo, or SRRM2 C-terminus Halo alone or fused to either TNPO3 or VCP.

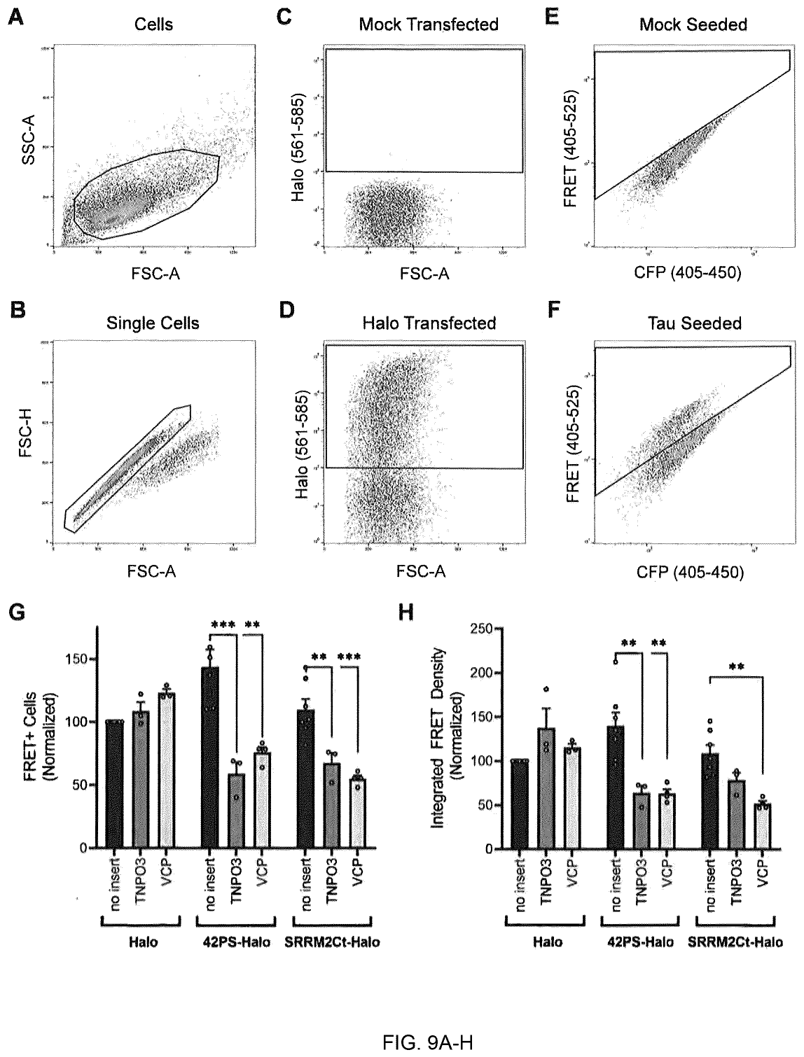

FIG. 10: Graphical abstract. Shows how cytoplasmic speckles are related to nuclear splicing speckles. Also shows how transfected tau seeds enter cells and can either be cleared by the proteasome of become recruited to cytoplasmic speckles where they associate with the polyserine repeats in SRRM2 and PNN, and grow into large cytoplasmic tau aggregates. The principles in this figure are employed to create novel fusion proteins that will be used to alter tau aggregation.

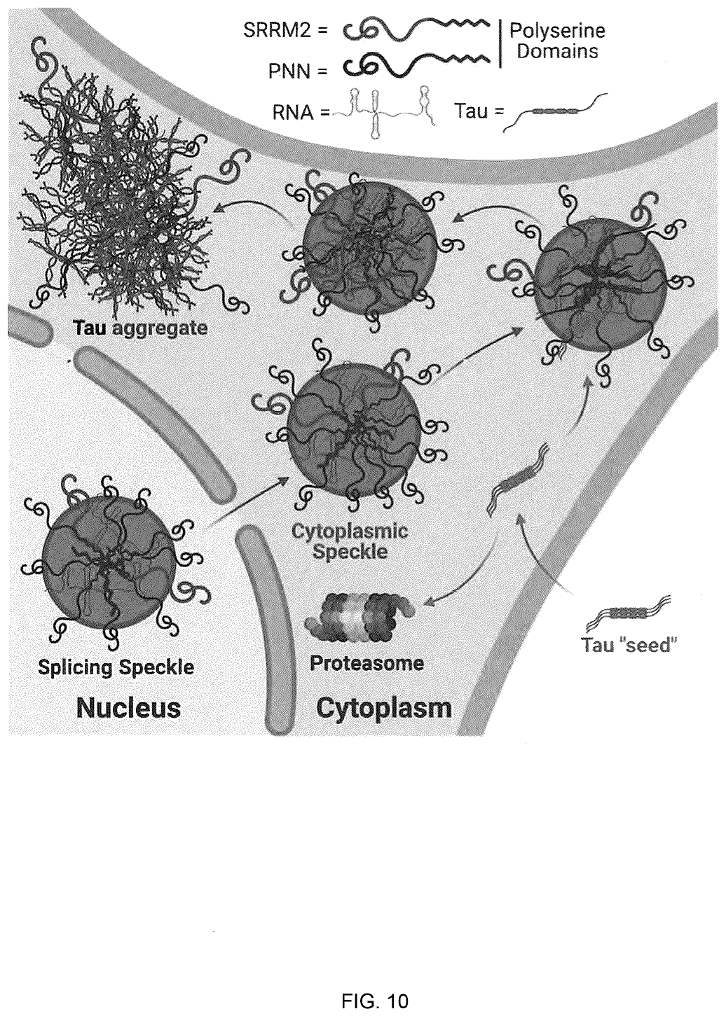

FIGS. 11A-P. Polyserine based motifs target a range of exogenous proteins to tau aggregates. (A) Schematic of constructs with C-terminal polyserine targeting motifs and Halo tags. (B) Schematic of constructs with N-terminal polyserine targeting motifs and Halo tags. (C-H) Immunofluorescence of DAPI (blue), Tau-YFP (green) and Halo (magenta) in HEK293T tau biosensor cells transfected with Halo, 42-polyserine (42PS) or SRRM2-Ct constructs without an upstream ORF (C), HSPA8 (D), LC3B (E), TNPO1 (F), TNPO3 (G), and VCP (H) as depicted in (A-B). (I-N) Quantification of the fold enrichment of Halo signal in cytoplasmic tau aggregates relative to the remainder of the cytoplasm in cells transfected with Halo, 42PS, or SRRM2-Ct without an upstream ORF (I), HSPA8 (J), LC3B (K), TNPO1 (L), TNPO3 (M), and VCP (N). (O) Integrated FRET density of the top 10% of Halo expressing cells measured via flow cytometry of HEK293T biosensor cells following 24 hours pre-transfection of Halo, 42PS or SRRM2-Ct tagged constructs without an upstream ORF or with TNPO3 or VCP at 12 hours post-seeding with clarified tau brain homogenate. (P) Integrated FRET density of the top 10% of Halo expressing cells measured via flow cytometry of HEK293T biosensor cells following 24 hours pre-transfection of Halo, 42PS or SRRM2-Ct tagged constructs without an upstream ORF or with TNPO3 or VCP at 24 hours post-seeding with clarified tau brain homogenate.

FIGS. 12A-K. UBXD8/FAF2 effectively ameliorates tau aggregation in a specific and targeting dependent manner. (A) Integrated FRET density of the top 10% of Halo expressing cells measured via flow cytometry of HEK293T biosensor cells following 24 hours pre-transfection of Halo, 42PS or SRRM2-Ct tagged constructs without an upstream ORF or with UBXD2, UBXD7 or UBXD8/FAF2 at 24 hours post-seeding with clarified tau brain homogenate. (B) Immunofluorescence of DAPI (blue), Tau-YFP (green) and Halo (magenta) in HEK293T tau biosensor cells co-transfected with clarified tau brain homogenate and untargeted FAF2-Halo or targeted FAF2-42PS-Halo. (C) Quantification of the fold enrichment of Halo signal in cytoplasmic tau aggregates in HEK293T tau biosensor cells transfected with Halo, 42PS-Halo, FAF2-Halo and FAF2-42PS-Halo and clarified tau brain homogenate. (D) Median YFP intensity as a measure of tau levels following gating for the top 10% of Halo expressing cells that are FRET− (no aggregates). (E) Median YFP intensity as a measure of tau levels following gating for the top 10% of Halo expressing cells that are FRET+ (with aggregates). (F) Representative Western blot for Halo, Tau-YFP, and GAPDH in HEK293T tau biosensor cells transfected with Halo, 42PS-Halo, FAF2-Halo and FAF2-42PS-Halo constructs. (G) Quantification of tau levels as a percent relative to Halo-transfected controls, measured by Western blot and normalized to GAPDH in groups as in (A). (H) Schematic of experimental timeline for (I). (I) Integrated FRET density of HEK293T tau biosensor cells seeded with cell extracts from HEK293T tau biosensor cells pre-transfected with denoted constructs for 24 hours, seeded with clarified tau brain homogenate, and collected 24 hours post-seeding as detailed in (F). (J) Schematic of experimental timeline for (K). (K) Integrated FRET density of the top 10% of Halo expressing HEK293T tau biosensor cells seeded with clarified tau brain homogenate and after 24 hours transfected with Halo, 42PS-Halo, FAF2 Halo and FAF2-42PS-Halo constructs at 24 and 48 hours post-seeding.

FIGS. 13A-I. Suppression of tau aggregation by UBXD8/FAF2 requires ubiquitin binding, monotopic hairpin, and VCP-binding domains. (A) Schematic of FAF2 domains and on formation at the ER membrane. (B) Western blot of Halo, Tau-YFP and GAPDH in HEK293T biosensor cells either untransfected, or transfected with Halo, 42PSHalo or FAF2 full-length or deletion constructs. (C) Schematic detailing the deletion of annotated FAF2 domains. (D) Immunofluorescence of DAPI (blue) and Halo (grey) in HEK293T tau biosensor cells transfected with FAF2 deletion mutants. (E) Integrated FRET density of the top 10% of HEK293T tau biosensor cells pre-transfected with Halo, 42PS-Halo and FAF2-42PS-Halo full-length or deletion mutants as described in (B-D) and seeded with clarified tau brain homogenate. (F) Schematic of FAF2min construct with deletion of both the UAS and coiled coil domains. (G) Immunofluorescence of DAPI (blue) and Halo (grey) in HEK293T tau biosensor cells transfected with Halo, 42PS or untargeted and targeted FAF2min constructs without or with polyserine, respectively. (H) Western blot of Halo, Tau-YFP and GAPDH in HEK293T biosensor cells either untransfected, or transfected with Halo, 42PS-Halo or FAF2 full-length or deletion constructs. (I) Integrated FRET density of the top 10% of HEK293T tau biosensor cells pre-transfected with Halo, 42PS-Halo and FAF2min either targeted or untargeted as described in (F-H) and seeded with clarified tau brain homogenate.

FIGS. 14A-P. Polyserine fusion proteins to do not modulate tau levels. (A) Amino acid sequence of the SRRM2-Ct targeting region which contains two polyserine stretches (blue). (B-C) Schematic and Western blot for Halo, Tau-YFP and GAPDH as a loading control of extracts from HEK293T tau biosensor cells transfected with Halo, 42PS and SRRM2-Ct constructs without an upstream ORF. Expected molecular weights are depicted. (D-E) Schematic and Western blot for Halo, Tau-YFP and GAPDH as a loading control of extracts from HEK293T tau biosensor cells transfected with constructs expressing Halo, 42PS and SRRM2-Ct tagged HSPA8. Expected molecular weights are depicted. (F-G) Schematic and Western blot for Halo, Tau-YFP and GAPDH as a loading control of extracts from HEK293T tau biosensor cells transfected with constructs expressing Halo, 42PS and SRRM2-Ct tagged LC3B. Expected molecular weights are depicted. (H-I) Schematic and Western blot for Halo, Tau-YFP and GAPDH as a loading control of extracts from HEK293T tau biosensor cells transfected with constructs expressing Halo, 42PS and SRRM2-Ct tagged TNPO1. Expected molecular weights are depicted. (J-K) Schematic and Western blot for Halo, Tau-YFP and GAPDH as a loading control of extracts from HEK293T tau biosensor cells transfected with constructs expressing Halo, 42PS and SRRM2-Ct tagged TNPO3. Expected molecular weights are depicted. (L-M) Schematic and Western blot for Halo, Tau-YFP and GAPDH as a loading control of extracts from HEK293T tau biosensor cells transfected with constructs expressing Halo, 42PS and SRRM2-Ct tagged VCP. Expected molecular weights are depicted. (N-P) Integrated FRET density of the top 10% of Halo expressing cells measured via flow cytometry of HEK293T biosensor cells following simultaneous transfection of Halo, 42PS or SRRM2-Ct tagged constructs without an upstream ORF or with HSPA8, LC3B, TNPO1, TNPO3 or VCP and clarified tau brain homogenate at 24 hours post-seeding.

### DETAILED DESCRIPTION OF THE INVENTION

The present invention includes novel methods and compositions for targeting tau aggregates, or components thereof. In one specific embodiment, the invention include the novel use of polyserine repeat sequences, and/or serine-rich sequences, which may be coupled with a heterologous peptide, to target tau aggregates, or components thereof, and thereby localize the heterologous peptide to the tau aggregate.

The present invention further includes novel methods and compositions for targeting a tau aggregate in a cell in an in vitro, or in vivo environment. In a preferred embodiment, the composition of the invention may include a fusion peptide having at least a first and second domain, which may, in some embodiment be coupled by a linker, such as a linker peptide. The first domain of the fusion peptide of the invention may include a tau targeting motif comprising an amino acid sequence encoding at least one polyserine repeat, or serine-rich sequence configured to target a tau binding motif on a tau aggregate. Notably, as used herein, a polyserine repeat of the invention may include a homologous series of serine residues, or may include non-consecutive serine repeat sequences, which may include a heterologous peptide domain having between 50-99% serine residues in the domain.

The tau binding motif of the invention may include a binding motif formed by one or more components of a tau aggregate, such as a monomeric tau peptide, or other nucleic acid or peptide, which may allow the first domain having a polyserine repeat of the invention to directly bind to the aggregate. In another embodiment, the tau binding motif of the invention may include a binding motif formed by one or more intermediate molecules, and preferably an endogenously expressed intermediate molecule such as a peptide or nucleic acid, configured to bind a tau aggregate or a component thereof, which may allow the first domain having a polyserine repeat of the invention to indirectly bind to the aggregate.

As noted above, the polyserine repeat of the invention may include a homologous polyserine repeat of: i) between 10-20 consecutive serine residues; ii) at least 20 consecutive serine residues; iii) between 20 and 42 consecutive serine residues; iv) between 20 and 50 consecutive serine residues; and at least 50 or more consecutive serine residues. In a preferred embodiment, the polyserine repeat of the invention may include a first domain containing a homologous polyserine repeat between 20 and 42 consecutive serine residues. In alternative embodiments, the polyserine repeat of the invention may include one or more non-consecutive serine repeat domains. In this embodiment, a non-consecutive serine repeat domain of the invention may include a serine enriched domain having a higher number of serine residues that the average number of serine incorporated into the peptides of a target organism. In a preferred embodiment, a non-consecutive serine repeat domain of the invention may include a peptide domain having between 83-99% serine residues, or alternatively a peptide domain having between 50-99% serine residues.

The polyserine repeat of the first domain of a fusion peptide of the invention may include a polyserine repeat from the C-terminal portion of Serine/Arginine Repetitive Matrix 2 (SRRM2) peptide (SEQ ID NO. 1), or a peptide or fragment containing a polyserine repeat of PNN (SEQ ID NO. 24), or a peptide or fragment containing a or one or more polyserine repeats of SETDIA (SEQ ID NO. 25), or a fragment or variant thereof. In this embodiment, the C-terminal portion of SRRM2 having one or more polyserine repeat may be engineered to be expressed as a fusion peptide with a heterologous peptide activity toward a tau aggregate or a component thereof as described below. In a preferred embodiment, the C-terminal portion of SRRM2 comprises a peptide encoding amino acids 2458-2752 of SEQ ID NO. 1, or alternatively a peptide encoding amino acids 2606-2752 of SEQ ID NO. 1, or alternatively a peptide encoding amino acids 2458-2606 of SEQ ID NO. 1. In a preferred embodiment, the C-terminal portion of SRRM2 comprises the amino acid sequence according to SEQ ID NO. 40, or 43, or fragment or variant thereof.

In one embodiment, the fusion peptide of the invention may be expresses in a cell, such as a human cell, or may be expressed in a protein production expression system, such as a bacterial, yeast, or other cellular culture production system and further isolated and/or purified. In this preferred embodiment, the fusion peptide of the invention may be incorporated into an as expression vector encoding a nucleotide sequence, operably linked to a promoter, for said fusion peptide. In alternative embodiment, the fusion peptide of the invention may be incorporated into an expression vector, such as a viral vector such as an adeno-virus vector, and delivered to the cells, and preferably the neural cells of a subject in need thereof.

The second domain of the fusion peptide of the invention may include heterologous peptide, or fragment thereof, having activity toward a tau aggregate or a component thereof, and preferably monomeric and/or pathogenic tau aggregates within a target cell. In a preferred embodiment, the heterologous peptide may inhibit the formation of pathogenic activity of the tau aggregate, preferably in a subject in need thereof, or in an in vitro environment, such as in a diagnostic assay and the like.

The heterologous peptide may be selected from the group consisting of, but not limited to a preferred number of heterologous peptides including: a peptide marker, a tag, E3 ubiquitin ligase, valosin-containing protein (VCP), transportin-3, and RNase enzyme, RNaseL, nucleases, a ligand, a radioligand, fluorescent ligand, a kinases, a phosphatases, and acetyl transferases, a ubiquitin ligase, a ubiquitin segregase, a fluorophore, a VCP adaptor, or a combination of the same. In this preferred embodiment, the activity of the heterologous peptide may include, but not be limited to: post-translational modification status of said tau aggregate, degrade a tau aggregate, decrease the activity of a tau aggregate, solubilize a tau aggregate, inhibit formation of a tau aggregate, inhibit the association of endogenous molecules with a tau aggregate.

The present invention further includes systems, methods, and compositions for targeting, tau aggregates, and preferably pathogenic tau aggregates in a cell. In a preferred embodiment, the invention includes a fusion peptide configured to target tau aggregates in a cell, or an intermediate molecule that binds a tau aggregate or a component thereof. The fusion peptide of the invention preferably includes a first domain comprising a tau targeting motif, preferably comprising an amino acid sequence encoding at least one polyserine repeat targeting a tau binding motif on a tau aggregate, or component thereof. The fusion peptide of the invention can include a second domain, linked to the first by, for example a linker and preferably a peptide linker, the second domain encoding a heterologous peptide, or fragment thereof, having activity toward the tau aggregate, or component thereof.

As noted above, the polyserine repeat sequence of the first domain can include one or more domains containing consecutive serine residues. In a preferred embodiment, the polyserine repeat sequence of the first domain can include between 10-50 consecutive serine residues, and variations of the same within this range. For example, the polyserine repeat sequence of the first domain can include: one or more domains containing less than 20 consecutive serine residues one or more domains containing at least 20 consecutive serine residues; one or more domains between 20 and 42 consecutive serine residues; one or more domains between 20 and 50 consecutive serine residues; one or more domains containing at least 50 or more consecutive serine residues. In a preferred embodiment described herein, the polyserine repeat sequence of the invention comprises one or more domains containing a polyserine repeat between 20 and 42 consecutive serine residues. In a preferred embodiment, the polyserine repeat of the invention includes 25, or even more preferably 42 consecutive serine residues according to SEQ ID NO. 29, or the nucleotide sequence according to SEQ ID NO. 28.

As further noted below, the polyserine repeat of the invention can include a consecutive series of serine residues, or one or more non-consecutive serine repeat domains, having non-serine residues within the repeat domain. For example, in some embodiment, the polyserine repeat of the invention includes a non-consecutive serine repeat domain having at least 75% serine residues, while in other embodiments, the polyserine repeat of the invention includes a non-consecutive serine repeat domain having between 50-99% serine residues. In still further embodiments, the the polyserine repeat of the invention can include at least one polyserine repeat and at least one non-consecutive serine repeat as described herein.

In alternative embodiment, the polyserine repeat of the first domain of the fusion peptide can further a portion of a peptide containing series of consecutive or non-consecutive series residues. For example, in one embodiment, the polyserine repeat of the first domain of the fusion peptide can include: a peptide or fragment containing a polyserine repeat of Serine/Arginine Repetitive Matrix 2 (SRRM2); the C-terminal portion of SRRM2 containing a polyserine repeat; or a peptide or fragment containing a polyserine repeat of PNN, the C-terminal portion of a PNN containing a polyserine repeat; or a peptide or fragment containing a one or more polyserine repeats of SETD1A, or a fragment or variant thereof.

For example, in alternative specific embodiments, the polyserine repeat of the first domain of the fusion peptide can include: the C-terminal portion of SRRM2 comprising a peptide encoding amino acids 2458-2752 of SEQ ID NO. 1; the C-terminal portion of SRRM2 according to the amino acid sequence SEQ ID NO. 40, or 46; or the C-terminal portion of PNN according to the amino acid sequence SEQ ID NO. 27; or a peptide or fragment containing a one or more polyserine repeats derived from SETDIA according to the amino acid sequence SEQ ID NO. 25.

The second domain, as noted above includes a heterologous peptide having activity toward tau aggregates or components thereof. Notably, this activity can be directed to cell in vitro, ex vivo or in vivo, and may preferably include human cells. As generally used herein, activity may include identifying, visualizing, tagging, inhibiting formation or growth, or a reduction of tau aggregates size or numbers in a cell. As noted above, this activity can occur through direct interaction between tau aggregates and the heterologous peptide, or through interactions between the heterologous peptide of the second domain and an intermediate molecules that interacts with, and has activity towards tau aggregates. In this preferred embodiment, a heterologous peptide, or fragment thereof of the fusion peptides second domain can include, but not be limited to: a peptide marker, a tag, E3 ubiquitin ligase, valosin-containing protein (VCP), and RNase enzyme, transportin 3, RNaseL, nucleases, a ligand, a radioligand, fluorescent ligand, a kinases, a phosphatases, and acetyl transferases, a ubiquitin ligase, a ubiquitin segregase, a fluorophore, a VCP adaptor, or a combination of the same. As noted above, the heterologous peptide, when targeted to a tau aggregate by the tau targeting motif of the first domain causes one more ore of the following: a post-translational modification status of the tau aggregate, degrade said tau aggregate, decrease the activity of said tau aggregate, solubilize said tau aggregate, inhibit formation of a tau aggregate, inhibit the association of endogenous molecules with said tau aggregate.

In a preferred embodiment, the heterologous peptide of the second domain having activity toward tau aggregates or components thereof can include, but not be limited to, valosin-containing protein (VCP) (SEQ ID NO. 38), transportin-3 (TNPO3) (SEQ ID NO. 39), Fas associated factor family member 2 (FAF2) (SEQ ID NO. 44), or a fragment or variant thereof. In one preferred, the heterologous peptide of the second domain can include select domains of the peptide, while retaining activity towards tau aggregates. For example, in one embodiment, the heterologous peptide comprises a truncated peptide comprising the UBA, hairpin, and UBX domains of FAF2, (being generally referred to herein as FAF2Min) (SEQ ID NO. 45)

Additional embodiments of the invention include nucleotide sequences, operably linked to a promote encoding one or more of the fusion peptides of the invention, which may further be isolated. Additional embodiments of the invention include an expression vector encoding a nucleotide sequence, operably linked to a promoter, encoding a fusion peptide of the invention. Still further embodiments of the invention, include a composition comprising one or more isolated or purified fusion peptide of the invention.

The present invention further included methods of treating a disease or condition in a subject, and preferably a human subject having a condition related to the formation of tau aggregates, and in particular pathogenic tau aggregates. In this embodiment, the invention may include methods and compositions to use one or more or the fusion peptides having polyserine repeat sequences as described herein to target tau aggregates, and preferably tau aggregates in a subject in need thereof. Such embodiments could be widely applicable to multiple neurodegenerative diseases presented by a subject including, but not limited to: Alzheimer's Disease, Frontotemporal Dementia, Chronic Traumatic Encephalopathy, Primary Age Related Tauopathy, Subacute Sclerosing Pan-Encephalitis, Corticobasal Degeneration, Progressive Supranuclear Palsy, Postencephalitic Parkinsonism, Mixed Dementia, Creutzfeldt-Jakob disease, Lytico-boydig disease, Amyotrophic-lateral sclerosis, Parkinson's disease, and Tuberous Sclerosis.

In one embodiment, the invention includes method of treating one or more of the above referenced conditions comprising administering a therapeutically effective amount of a fusion peptide of the invention. This method of treatment may include administering a therapeutically effective amount of a pharmaceutical composition comprising one or more fusion peptides of the invention, and a pharmaceutically acceptable carrier.

Additional embodiments of the invention include novel methods and composition to inhibit tau aggregate formation in a cell. In a preferred embodiment, expression of PNN may be downregulated in a cell, and preferably the cell of a human subject, through one or more inhibitory RNA molecules, and in particular a small inhibitory RNA (siRNA) molecules directed to inhibit the expression of PNN in a target cell, having a sense strand selected from the group consisting of: SEQ ID NO.'s 41-43, which can be administered to a subject in need thereof as a pharmaceutical composition as defined herein.

Additional aspects of the invention include novel methods and composition to inhibit tau aggregate formation in a cell. In a preferred embodiment, expression of PNN may be downregulated in a cell, and preferably the cell of a human subject, through one or more inhibitory RNA molecules, and in particular a small inhibitory RNA (siRNA) molecules directed to inhibit the expression of PNN, having a sense strand selected from the group consisting of: SEQ ID NO.'s 41-43.

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y. and Current Protocols in Molecular Biology (Ausbel et al., eds., John Wiley & Sons, Inc. 2001. As appropriate, procedures involving the use of commercially available kits and reagents are generally carried out in accordance with manufacturer defined protocols and/or parameters unless otherwise noted.

As used herein, a “serine repeat” or “polyserine repeat” means a peptide domain containing a homologous regions of consecutive serine residues or a domain containing non-consecutive serine repeats. A “non-consecutive serine repeat” means a peptide domain containing a heterologous region wherein the majority of resides in the domain are serine residues, or higher than the average number of serine residues present when compared to the average incorporation of serine in a wild-type peptide. In certain embodiments a “serine repeat” or serine-rich repeat” may be a wild-type or engineered sequence, or may be part of a fusion peptide.

As used herein, a “moiety” or “motif” comprises an amino acid, peptide, polypeptide, sugar, nucleic acid or other biological molecule having a structure that can be recognized and bind with another molecule. As used herein, a “tau aggregate moiety” or “tau aggregate motif” comprises an peptide or biological molecule having one or more serine repeat domains that can be recognized and bind directly, or indirectly with a tau aggregate, or a portion of a tau aggregate thereof.

The terms “inhibit” and “reduce” or grammatical variations thereof as used herein refer to a decrease or diminishment in the specified level or activity of at least about 15%, 25%, 35%, 40%, 50%, 60%, 75%, 80%, 90%, 95% or more. In particular embodiments, the inhibition or reduction results in little or essentially no detectible entity or activity (at most, an insignificant amount, e.g., less than about 10% or even 5%). In a preferred embodiment, the present invention “inhibits” the formation of tau aggregates in a call. In another embodiment, the present invention decreases the number of already formed tau aggregates in a cell. In another embodiment, the present invention decreases or inhibits the pathogenies of tau aggregates in a cell through the inhibition of their formation of reduction in already formed aggregates.

As used herein, “complex” means an assemblage or aggregate of molecules in direct or indirect contact with one another. As used herein, “contact,” or more particularly, “contacting” with reference to an individual or complex of molecules, means two or more molecules are close enough that weak noncovalent chemical interactions, such as Van der Waal forces, hydrogen bonding, ionic and hydrophobic interactions, and the like, dominate the interaction of the molecules Generally, a complex of molecules is stable in that under assay conditions the complex is thermodynamically more favorable than a non-aggregated state of its component molecules.

As used herein, the term “tauopathy” refers to a family of neurodegenerative diseases that may present in a subject, or to which a subject may be at risk of developing, being generally characterized by a malfunction of a tau protein (family closely related to intracellular microtubule-related proteins). These neurodegenerative diseases (tauopathy) include, for example, Alzheimer's disease, Parkinson's disease, progressive supranuclear palsy, corticobasal degeneration, argyrophilic grain diseases, Pick's disease and fronto-temporal dementia.

As used herein, the term “aggregate,” refers to a state in which various insoluble fibrous proteins are deposited and aggregated in a patient's tissue. In particular, the term “tau aggregate” means an aggregate formed by aggregation of tau fiber proteins, mainly involved in entanglement of nerve fibers.

The terms “administer,” “administering,” or “administration” refers to injecting, implanting, absorbing, or ingesting one or more therapeutic fusion peptides of the invention, which may be part of a pharmaceutical composition.

A “therapeutically effective amount” of a compound, preferably a therapeutic fusion peptide, of the present invention or a pharmaceutical composition thereof is an amount sufficient to provide a therapeutic benefit in the treatment of a disease or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, and/or enhances the therapeutic efficacy of another therapeutic agent. A “therapeutically effective amount” may also mean “prophylactically effective amount” of a compound of the present invention, such as a fusion peptide that is configured to target tau aggregates, or components thereof, is an amount sufficient to prevent a disease or one or more symptoms associated with the condition or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent.

A “pharmaceutical composition” or “pharmaceutical composition of the invention” refers to a composition of the invention, and preferably a therapeutic fusion peptide composition of the invention, or a pharmaceutically acceptable salt, solvate, hydrate or prodrug thereof as an active ingredient, and at least one pharmaceutically acceptable carrier or excipient. In some embodiments, the pharmaceutical composition comprises two or more pharmaceutically acceptable carriers and/or excipients. In other embodiments, the pharmaceutical composition further comprises at least one additional anticancer therapeutic agent, such as through a co-treatment. As used herein, a “pharmaceutically acceptable carrier” refers to a carrier or diluent that does not cause significant irritation to an organism and does not abrogate the biological activity and properties of the administered composition of the invention. The pharmaceutical acceptable carrier may comprise any conventional pharmaceutical carrier or excipient. The choice of carrier and/or excipient will to a large extent depend on factors such as the particular mode of administration, the effect of the carrier or excipient on solubility and stability, and the nature of the dosage form. As also used herein, a “pharmaceutical composition” may include additional mechanisms to deliver a fusion peptide of the invention to a target cell, such as a neuronal cell in a subject. In one embodiment, viral vectors, such as adenovirus vectors and subviral particles for fusion peptide delivery may be included within the definition of pharmaceutical compositions generally. See Kron M W, Kreppel F. Adenovirus vectors and subviral particles for protein and peptide delivery. Curr Gene Ther. 2012 October;12(5):362-73, for methods of peptide delivery by viral vectors, which is incorporated herein by reference)

Suitable pharmaceutical carriers include inert diluents or fillers, water, and various organic solvents (such as hydrates and solvates). The pharmaceutical compositions may, if desired, contain additional ingredients such as flavorings, binders, excipients, and the like. Thus, for oral administration, tablets containing various excipients, such as citric acid may be employed together with various disintegrants such as starch, alginic acid and certain complex silicates and with binding agents such as sucrose, gelatin, and acacia. Examples, without limitation, of excipients include calcium carbonate, calcium phosphate, various sugars and types of starch, cellulose derivatives, gelatin, vegetable oils and polyethylene glycols. Additionally, lubricating agents such as magnesium stearate, sodium lauryl sulfate and talc are often useful for tableting purposes. Solid compositions of a similar type may also be employed in soft and hard filled gelatin capsules. Non-limiting examples of materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

The pharmaceutical composition may, for example, be in a form suitable for oral administration as a tablet, capsule, pill, powder, sustained release formulations, solution suspension, for parenteral injection as a sterile solution, suspension, or emulsion, for topical administration as an ointment or cream or for rectal administration as a suppository. The pharmaceutical composition may be in unit dosage forms suitable for single administration of precise dosages.

Also encompassed by the invention are kits (e.g., pharmaceutical packs). The kits provided may comprise a therapeutic fusion peptide composition (e.g., pharmaceutical or diagnostic composition) and a container (e.g., a vial, ampule, bottle, syringe, and/or dispenser package, or other suitable container). The kits provided may comprise antibodies that selectively bind a therapeutic fusion peptide (e.g., pharmaceutical or diagnostic composition) and a container (e.g., a vial, ampule, bottle, syringe, and/or dispenser package, or other suitable container). In some embodiments, provided kits may optionally further include a second container comprising an excipient (e.g., pharmaceutically acceptable carrier) for dilution or suspension of an inventive pharmaceutical composition or compound. In some embodiments, the therapeutic fusion peptide composition provided in the first container and the second container are combined to form one unit dosage form.

The term “subject” refers to any animal. In certain embodiments, the subject is a mammal. In certain embodiments, the subject is a human (e.g., a man, a woman, or a child). The human may be of either sex, or may be at any stage of development. In certain embodiments, the subject has been diagnosed with the mitochondrial condition or disease to be treated. In other embodiments, the subject is at risk of developing the mitochondrial condition or disease. In certain embodiments, the subject is an experimental animal (e.g., mouse, rat, rabbit, dog, pig, or primate). The experimental animal may be genetically engineered. In certain embodiments, the subject is an animal (e.g., dog, cat, bird, horse, cow, goat, sheep, chicken, mule deer, white-tailed deer, red deer, elk, caribou, reindeer, sika deer, or moose).

As used herein, the phrase “in need thereof” means that the animal or mammal has been identified as having a need for the particular method or treatment. In some embodiments, the identification can be by any means of diagnosis. In any of the methods and treatments described herein, the animal or mammal can be in need thereof. In some embodiments, the animal or mammal is in an environment or will be traveling to an environment in which a particular disease, disorder, or condition is prevalent.

“Nucleic acid” refers to deoxyribonucleotides or ribonucleotides and polymers thereof in either single-or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless otherwise indicated, a particular nucleic acid sequence also implicitly encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); Rossolini et al., Mol. Cell. Probes 8:91-98 (1994)). The term nucleic acid is used interchangeably with gene, cDNA, mRNA, oligonucleotide, and polynucleotide.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “coupled” or “ligated” when applied to a peptide of the invention may include direct chemical bonds, such as covalent linkages, such as through the generation of fusion or chimera proteins. In alternative embodiment, couple term “coupled” when applied to a peptide of the invention may include instances where a peptide of the invention may be bound to another peptide or molecule through an intermediary compound or molecule, such as a peptide linker.

The terms “conjugating” or “linking” or “coupling” in the context of the present invention with respect to connecting two or more molecules or components to form a complex refers to joining or conjugating said molecules or components, e.g. proteins, via a covalent bond, particularly an isopeptide bond which forms between the peptides.

A “domain” refers to a unit of a protein or protein complex, comprising a polypeptide subsequence, a complete polypeptide sequence, or a plurality of polypeptide sequences where that unit has a defined function. The function is understood to be broadly defined and can be ligand binding, catalytic activity or can have a stabilizing effect on the structure of the protein.

As used herein, the term “wild-type” refers to a gene or gene product isolated from a naturally occurring source. A wild-type gene is that which is most frequently observed in a population and is thus arbitrarily designed the “normal” or “wild-type” form of the gene. In contrast, the term “modified” or “engineered” refers to a gene or gene product that displays modifications in sequence and/or functional properties (i.e., altered characteristics) when compared to the wild-type gene or gene product. It is noted that naturally occurring mutants can be isolated; these are identified by the fact that they have altered characteristics (including altered nucleic acid sequences) when compared to the wild-type gene or gene product.

The term “peptide tag” or “peptide linker” as used herein generally refers to a peptide or oligopeptide. There is no standard definition regarding the size boundaries between what is meant by peptide or oligopeptide but typically a peptide may be viewed as comprising between 2-20 amino acids and oligopeptide between 21-39 amino acids. Accordingly, a polypeptide may be viewed as comprising at least 40 amino acids, preferably at least 50, 60, 70 or 80 amino acids. Thus, a peptide tag or linker as defined herein may be viewed as comprising at least 12 amino acids, e.g. 12-39 amino acids, such as e.g. 13-35, 14-34, 15-33, 16-31, 17-30 amino acids in length, e.g. it may comprise or consist of 12, 13, 14, 15, 16, 17, 18, 19, 20, 21, 22 or 23 amino acids.

A “fusion” or “chimera” protein is a polypeptide produced when two heterologous nucleotide sequences or fragments thereof coding for two (or more) different polypeptides not found fused together in nature are fused together in the correct translational reading frame. As used herein, a “functional” polypeptide or “fragment” is one that substantially retains at least one biological activity normally associated with that polypeptide (e.g., nucleosome formation). In particular embodiments, the “functional” polypeptide or “fragment” substantially retains all of the activities possessed by the unmodified peptide. By “substantially retains” biological activity, it is meant that the polypeptide retains at least about 20%, 30%, 40%, 50%, 60%, 75%, 85%, 90%, 95%, 97%, 98%, 99%, or more, of the biological activity of the native polypeptide (and can even have a higher level of activity than the native polypeptide).

The term, “expression” or “expressing” refers to production of a functional product, such as, the generation of an RNA transcript from an introduced construct, an endogenous DNA sequence, or a stably incorporated heterologous DNA sequence. A nucleotide encoding sequence may comprise intervening sequence or may lack such intervening non-translated sequences (e.g., as in cDNA). Expressed genes include those that are transcribed into mRNA and then translated into protein and those that are transcribed into RNA but not translated (for example, siRNA, transfer RNA, and ribosomal RNA). The term may also refer to a polypeptide produced from an mRNA generated from any of the above DNA precursors. Thus, expression of a nucleic acid fragment, such as a gene or a promoter region of a gene, may refer to transcription of the nucleic acid fragment (e.g., transcription resulting in mRNA or other functional RNA) and/or translation of RNA into a precursor or mature protein (polypeptide), or both.

An “expression vector” or “vector” refers to a nucleic acid construct, which when introduced into a host cell, results in transcription and/or translation of a RNA or polypeptide, respectively. More specifically, the term “vector” refers to some means by which DNA, RNA, a protein, or polypeptide can be introduced into a host. The polynucleotides, protein, and polypeptide which are to be introduced into a host can be therapeutic or prophylactic in nature; can encode or be an antigen; can be regulatory in nature, etc. There are various types of vectors including virus, plasmid, bacteriophages, cosmids, and bacteria. Again, more specifically, “expression vector” is nucleic acid capable of replicating in a selected host cell or organism. An expression vector can replicate as an autonomous structure, or alternatively can integrate, in whole or in part, into the host cell chromosomes or the nucleic acids of an organelle, or it is used as a shuttle for delivering foreign DNA to cells, and thus replicate along with the host cell genome. Thus, an expression vector are polynucleotides capable of replicating in a selected host cell, organelle, or organism, e.g., a plasmid, virus, artificial chromosome, nucleic acid fragment, and for which certain genes on the expression vector (including genes of interest) are transcribed and translated into a polypeptide or protein within the cell, organelle or organism; or any suitable construct known in the art, which comprises an “expression cassette.”

In contrast, as described in the examples herein, a “cassette” is a polynucleotide containing a section of an expression vector of this invention. The use of the cassettes assists in the assembly of the expression vectors. An expression vector is a replicon, such as plasmid, phage, virus, chimeric virus, or cosmid, and which contains the desired polynucleotide sequence operably linked to the expression control sequence(s). A polynucleotide sequence is operably linked to an expression control sequence(s) (e.g., a promoter and, optionally, an enhancer) when the expression control sequence controls and regulates the transcription and/or translation of that polynucleotide sequence.

A “variant,” or “isoform,” or “protein variant” is a member of a set of similar proteins that perform the same or similar biological roles. For example, fragments and variants of the disclosed polynucleotides and amino acid sequences of the invention encoded thereby are also encompassed by the present invention. By “fragment” is intended a portion of the polynucleotide or a portion of the amino acid sequence. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. In certain preferred embodiments, a variant may include a polynucleotide having between 80-99% homology to the reference polynucleotide, while retaining the described. function.

As used herein, a “native” or “wildtype” polynucleotide or polypeptide comprises a naturally occurring nucleotide sequence or amino acid sequence, respectively. In one embodiment, a “fragment,” as applied to a polypeptide, will be understood to mean an amino acid sequence of reduced length relative to a reference polypeptide or amino acid sequence and comprising, consisting essentially of, and/or consisting of an amino acid sequence of contiguous amino acids identical or almost identical (e.g., at least 90%, 92%, 95%, 98%, 99% identical) to the reference polypeptide or amino acid sequence. Such a polypeptide fragment according to the invention may be, where appropriate, included in a larger polypeptide of which it is a constituent. In some embodiments, such fragments can comprise, consist essentially of, and/or consist of peptides having a length of at least about 4, 6, 8, 10, 12, 15, 20, 25, 30, 35, 40, 45, 50, 75, 100, 150, 200, or more consecutive amino acids of a polypeptide or amino acid sequence according to the invention. In some embodiments, such fragments can comprise, consist essentially of, and/or consist of peptides having a length of less than about 4, 6, 8, 10, 12, 15, 20, 25, 30, 35, 40, 45, 50, 75, 100, 150, or 200 consecutive amino acids of a polypeptide or amino acid sequence according to the invention.

The term “fragment,” as applied to a polynucleotide, can further be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., at least 90%, 92%, 95%, 98%, 99% identical) to the reference nucleic acid or nucleotide sequence, wherein the fragment retains the function of the full polynucleotide. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the invention. In some embodiments, such fragments can comprise, consist essentially of, and/or consist of oligonucleotides having a length of less than about 8, 10, 12, 15, 20, 25, 30, 35, 40, 45, 50, 75, 100, 150, or 200 consecutive nucleotides of a nucleic acid or nucleotide sequence according to the invention.

The term “gene” or “nucleotide sequence” refers to a coding region operably joined to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF) as well as, where applicable, intervening sequences (i.e., introns) between individual coding regions (i.e., exons). The term “gene” or “nucleotide sequence” as used herein can mean a DNA sequence that is transcribed into mRNA which is then translated into a sequence of amino acids characteristic of a specific polypeptide.

The term “heterologous” or “exogenous” refers to a nucleic acid fragment or protein that is foreign to its surroundings. In the context of a nucleic acid fragment, this is typically accomplished by introducing such fragment, derived from one source, into a different host. Heterologous nucleic acid fragments, such as coding sequences that have been inserted into a host organism, are not normally found in the genetic complement of the host organism. As used herein, the term “heterologous” also refers to a nucleic acid fragment derived from the same organism, but which is located in a different, e.g., non-native, location within the genome of this organism. A nucleic acid fragment that is heterologous with respect to an organism into which it has been inserted or transferred is sometimes referred to as a “transgene.”

The term “endogenous” refers to a component naturally found in an environment, i.e., a gene, nucleic acid, miRNA, protein, cell, or other natural component expressed in the subject, as distinguished from an introduced component, i.e., an “exogenous” component.

Unless otherwise stated, nucleic acid sequences in the text of this specification are given, when read from left to right, in the 5′ to 3′ direction. Nucleic acid sequences may be provided as DNA or as RNA, as specified; disclosure of one necessarily defines the other, as is known to one of ordinary skill in the art and is understood as included in embodiments where it would be appropriate. Nucleotides may be referred to by their commonly accepted single-letter codes. Unless otherwise indicated, amino acid sequences are written left to right in amino to carboxyl orientation, respectively. Amino acids may be referred to herein by either their commonly known three letter symbols or by the one-letter symbols as generally understood by those skilled in the relevant art.

As used herein, “operably linked” refers to a functional arrangement of elements. A first nucleic acid sequence is operably linked with a second nucleic acid sequence when the first nucleic acid sequence is placed in a functional relationship with the second nucleic acid sequence. For instance, a promoter is operably linked to a coding sequence if the promoter effects the transcription or expression of the coding sequence. The control elements need not be contiguous with the coding sequence, so long as they function to direct the expression thereof. Thus, for example, intervening untranslated yet transcribed sequences can be present between a promoter and the coding sequence and the promoter can still be considered “operably linked” to the coding sequence. The term “promoter” or “regulatory element” refers to a region or nucleic acid sequence located upstream or downstream from the start of transcription and which is involved in recognition and binding of RNA polymerase and/or other proteins to initiate transcription of RNA. Promoters useful in the present methods include, for example, constitutive, strong, weak, tissue-specific, cell-type specific, seed-specific, inducible, repressible, and developmentally regulated promoters.

As used herein, the term “transformation” or “genetically modified” refers to the transfer of one or more nucleic acid molecule(s) into a cell. A microorganism is “transformed” or “genetically modified” by a nucleic acid molecule transduced into the bacteria when the nucleic acid molecule becomes stably replicated by the bacteria. As used herein, the term “transformation” or “genetically modified” encompasses all techniques by which a nucleic acid molecule can be introduced into, such as a bacterium.

The term “antibody,” as used herein, refers to an immunoglobulin, e.g., an antibody, and to antigen binding portions thereof, e.g., molecules that contain an antigen binding site which specifically binds an antigen, such as a polypeptide. A molecule which specifically binds to a given polypeptide, but does not substantially bind other molecules in a sample, e.g., a biological sample, which naturally contains the polypeptide. Antibody molecules include “antibody fragments” which refers to a portion of an intact antibody that is sufficient to confer recognition and specific binding to a target antigen. Examples of antibody fragments include, but are not limited to, Fab, Fab′, F(ab′)2, and Fv fragments, linear antibodies, scFv antibodies, a linear antibody, single domain antibody (sdAb), e.g., either a variable light (VL) chain or a variable heavy (VH) chain, a camelid VHH domain, and multispecific antibodies formed from antibody fragments. Antibody molecules can be polyclonal or monoclonal. The term “monoclonal” as applied to antibody molecules herein, refers to a population of antibody molecules that contain only one species of an antigen binding site capable of immunoreacting with a particular epitope.

A “marker,” or “tag” as used herein, refers to a molecule that can be used for identification, detection, purification, or isolation. In an embodiment, the marker comprises a small molecule, a peptide, a polypeptide, or a labeled amino acid or nucleotide. In an embodiment, the marker generates a signal for detection, e.g., a radioactive signal, a chemiluminescent signal, a fluorescent signal, or a chromogenic signal. For example, the marker is a dye, a fluorophore, a reporter enzyme (e.g., a photoprotein, luciferase), a fluorescent peptide, or a radionuclide. The generated signal can be detected by a variety of assays known in the art, such as fluorescence microscopy, fluorescence-activated cell sorting, gel electrophoresis, and spectrophotometry.

Downregulating expression of a gene such as PNN can be monitored, for example, by direct detection of gene transcripts (for example, by PCR), by detection of polypeptide(s) encoded by the gene (for example, by Western blot or immunoprecipitation), by detection of biological activity of polypeptides encode by the gene (for example, catalytic activity, ligand binding, and the like), or by monitoring changes in the hosts (for example, reduced mortality of the host etc.). Additionally or alternatively downregulating expression of a pathogen resistance gene product may be monitored by measuring pathogen levels (e.g. viral levels, bacterial levels etc.) in the host as compared to wild type (i.e. control) hosts not treated by the agents of the invention.

The term “siRNA” refers to small inhibitory RNA duplexes (generally between 17-30 base pairs, but also longer e.g., 31-50 bp) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC. It has been found that position of the 3′-overhang influences potency of a siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The terms “isolated”, “purified”, or “biologically pure” as used herein, refer to material that is substantially or essentially free from components that normally accompany the material in its native state or when the material is produced. In an exemplary embodiment, purity and homogeneity are determined using analytical chemistry techniques such as polyacrylamide gel electrophoresis or high performance liquid chromatography. A nucleic acid or particular bacteria that are the predominant species present in a preparation is substantially purified. In an exemplary embodiment, the term “purified” denotes that a nucleic acid or protein gives rise to essentially one band in an electrophoretic gel. Typically, isolated nucleic acids or proteins have a level of purity expressed as a range. The lower end of the range of purity for the component is about 60%, about 70% or about 80% and the upper end of the range of purity is about 70%, about 80%, about 90% or more than about 90%.

As used herein the singular forms “a,” “an,” and “the” include plural referents unless the context clearly dictates otherwise. Thus, for example, reference to “a cell” includes one or more cells and equivalents thereof known to those skilled in the art, and so forth. Similarly, the word “or” is intended to include “and” unless the context clearly indicates otherwise. Hence “comprising A or B” means including A, or B, or A and B. Furthermore, the use of the term “including,” as well as other related forms, such as “includes” and “included,” is not limiting.

The term “about” as used herein is a flexible word with a meaning similar to “approximately” or “nearly.” The term “about” indicates that exactitude is not claimed, but rather a contemplated variation. Thus, as used herein, the term “about” means within 1 or 2 standard deviations from the specifically recited value, or +a range of up to 20%, up to 15%, up to 10%, up to 5%, or up to 4%, 3%, 2%, or 1% compared to the specifically recited value.

The invention described herein suitably may be practiced in the absence of any element(s) not specifically disclosed herein. Thus, for example, in each instance herein any of the terms “comprising,” “consisting essentially of,” and “consisting of” may be replaced with either of the other two terms.

The invention now being generally described will be more readily understood by reference to the following examples, which are included merely for the purposes of illustration of certain aspects of the embodiments of the present invention. The examples are not intended to limit the invention, as one of skill in the art would recognize from the above teachings and the following examples that other techniques and methods can satisfy the claims and can be employed without departing from the scope of the claimed invention. Indeed, while this invention has been particularly shown and described with references to preferred embodiments thereof, it will be understood by those skilled in the art that various changes in form and details may be made therein without departing from the scope of the invention encompassed by the appended claims.

### EXAMPLES

### Example 1: Rationale, Summary and Application of Inventive Features

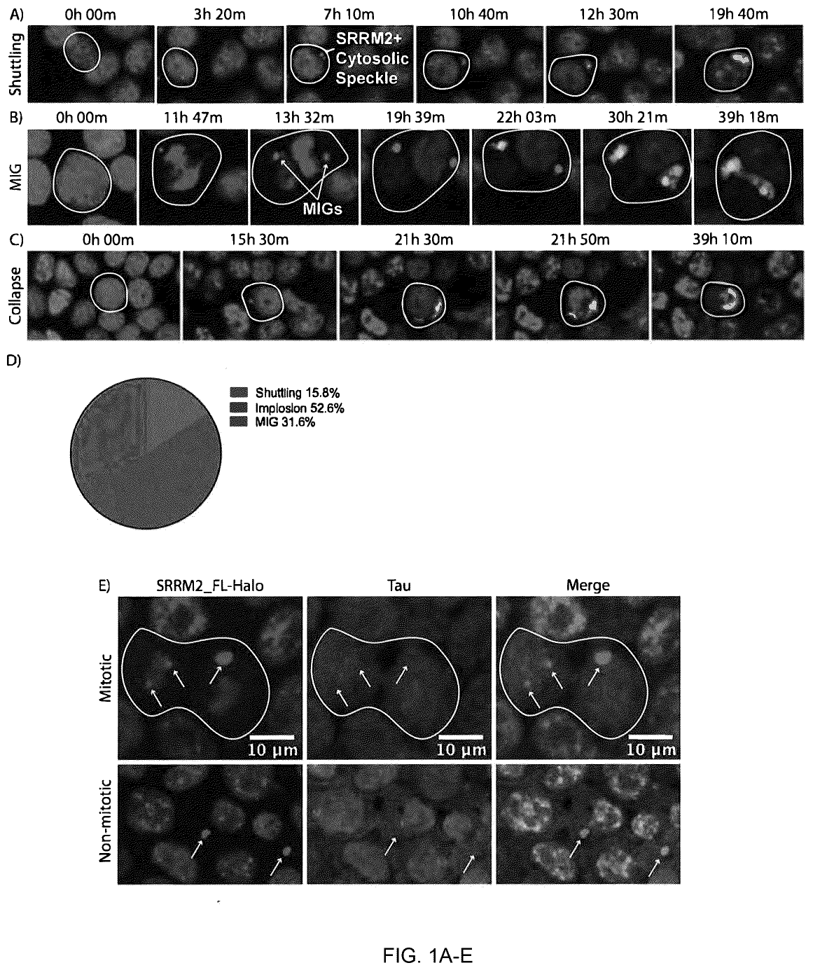

The present inventors further describe the interaction of Serine/Arginine Repetitive Matrix 2 (SRRM2) with tau aggregates that is affected by several inputs that regulate its partitioning into the cytoplasm. In HEK293 cells, Applicants found that SRRM2 (and possibly other tau interacting RBPs) can accumulate in the cytoplasm through three distinct events. The first mechanism is export of SRRM2 from the nucleus without cell division where it forms cytoplasmic assemblies that can nucleate tau aggregates from their surface (FIG. 1A). The second is mitosis where SRRM2 forms MIGs that can nucleate or merge with tau aggregates (FIG. 1B). The third mechanism is cellular implosion where there is chromatin condensation, and a rapid association of SRRM2 with tau aggregates (FIGS. 1C, D).

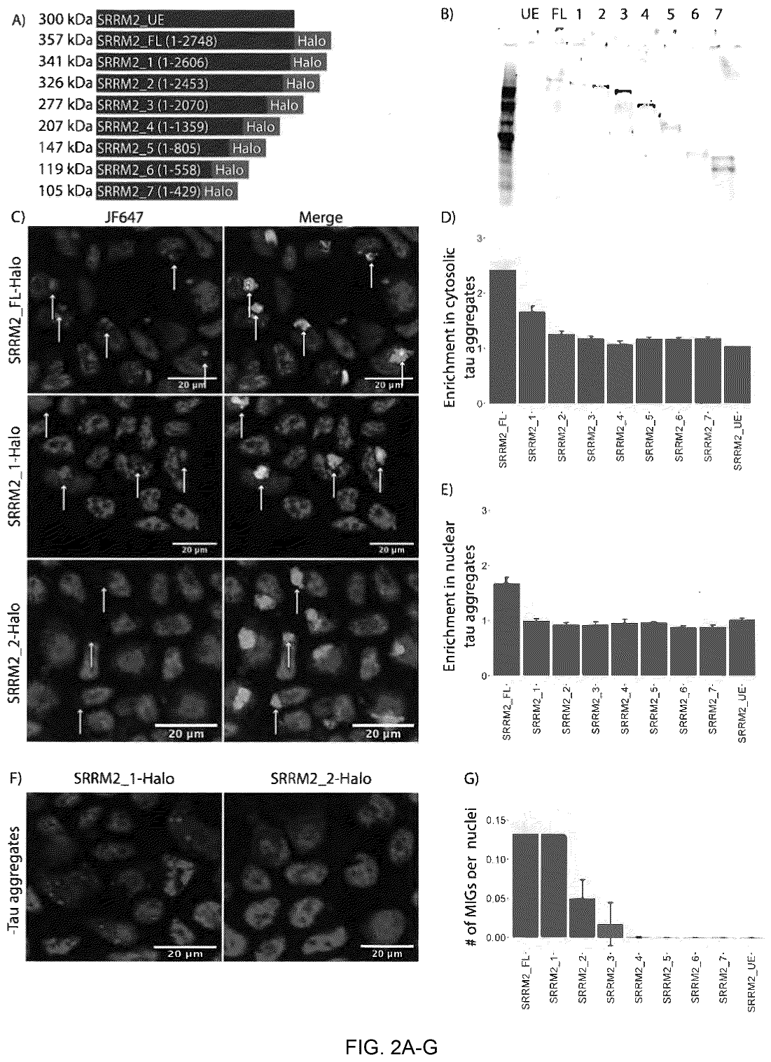

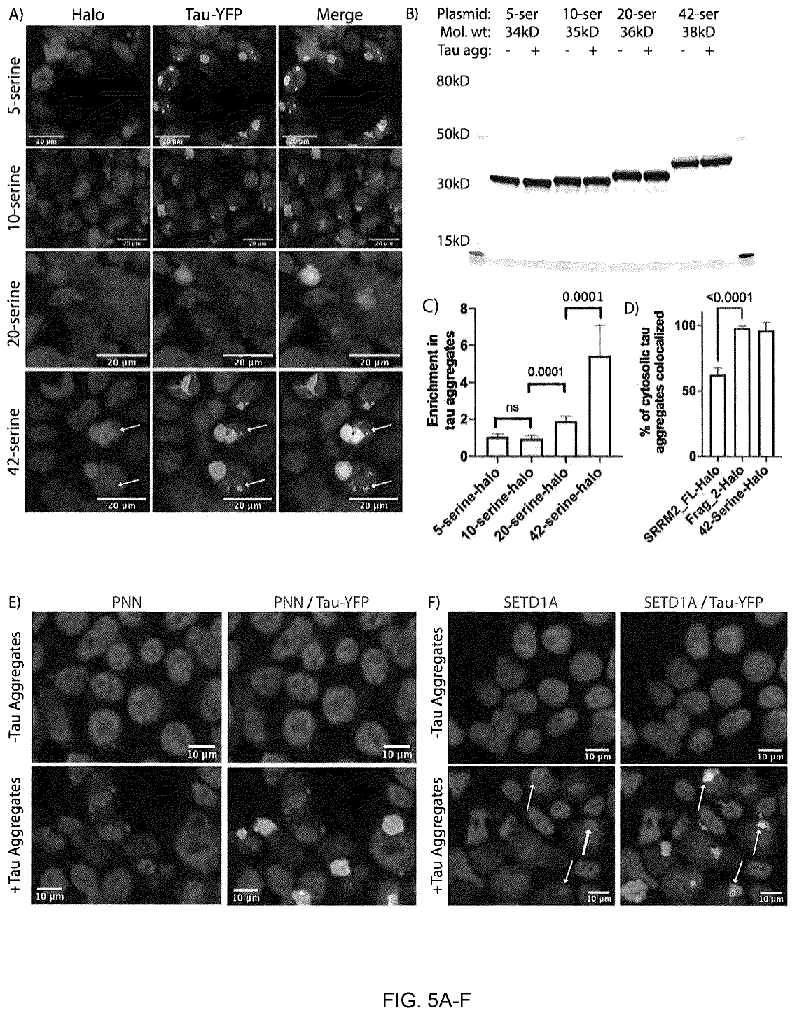

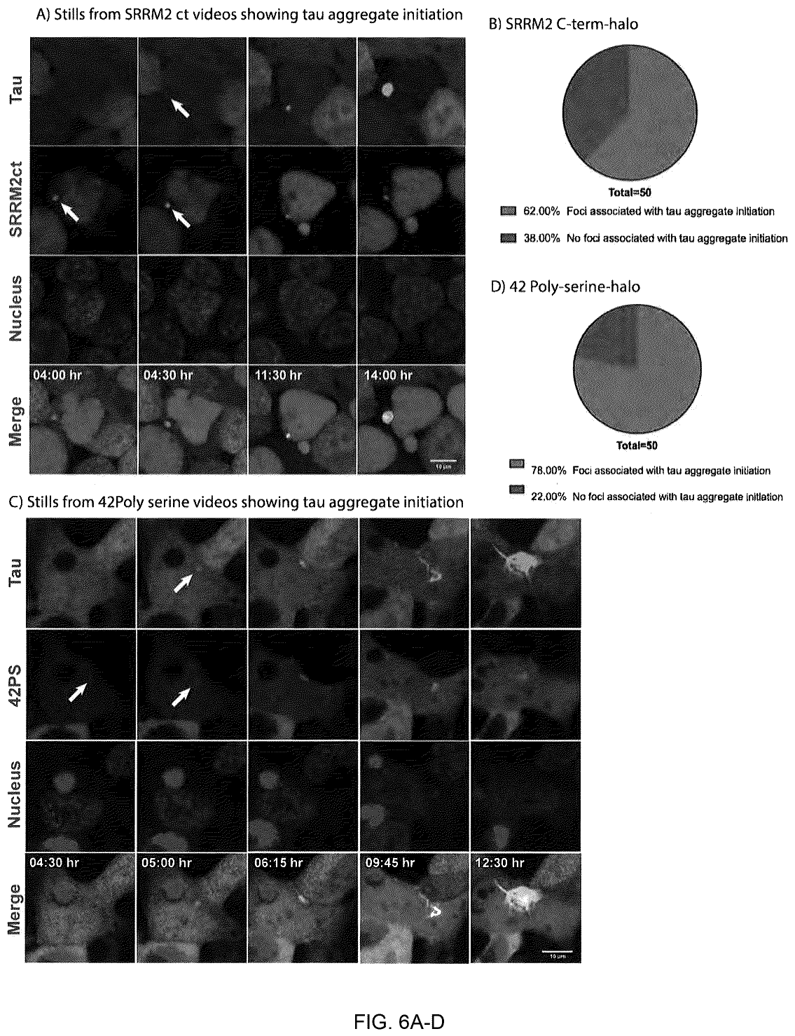

The present inventors further demonstrate that a polyserine run is sufficient to target proteins to both cytoplasmic and nuclear tau aggregates. This conclusion is based on the following observations. First, the present inventors observed that two regions of the C terminal domain of SRRM2 that contain polyserine repeats influence its recruitment to tau aggregates (FIGS. 2, 3, 4). Second, the addition of 42 serines, or 20 serines, is sufficient to target the Halo protein to tau aggregates, with longer repeats showing greater recruitment (FIGS. 5A-C). Moreover, expression of SRRM2 C-terminus-halo and 42-polyserine in HEK293 tau biosensor cells showed that both constructs created cytoplasmic assemblies that were associated with more than 62% of tau aggregation initiation events (FIG. 6). This suggests that polyserine creates an environment that recruits aggregated tau.

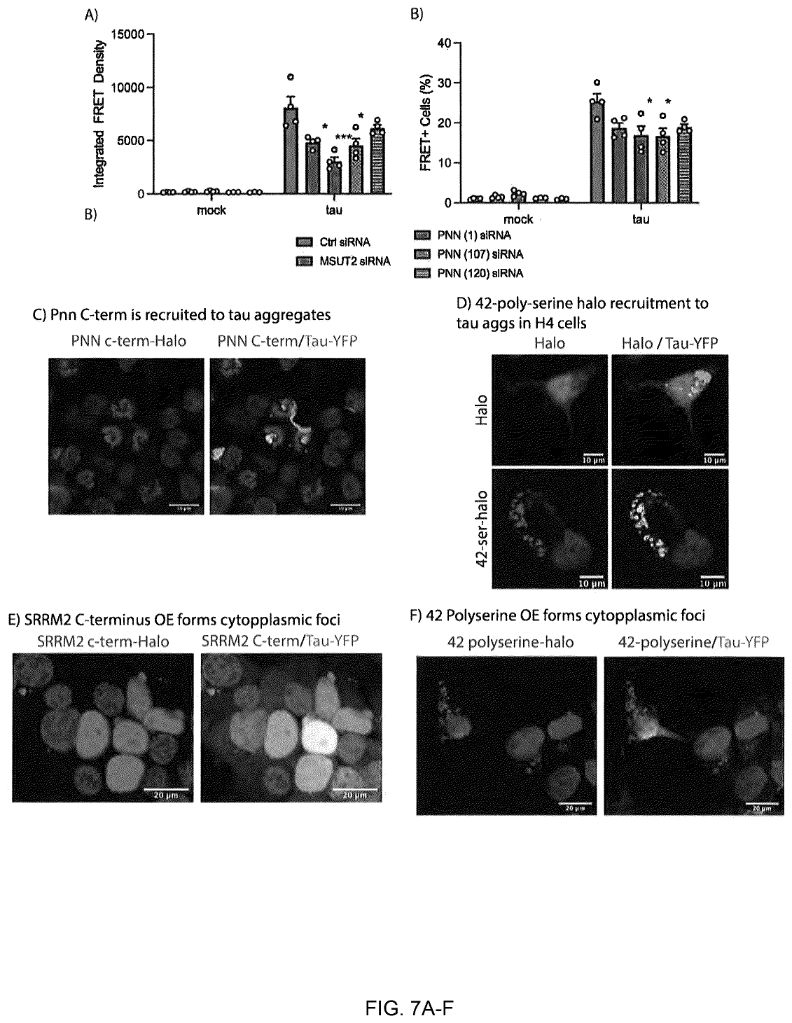

Given that the tau microtubule repeat binding domains (MTBD) are positively charged, polyserine repeats may be heavily phosphorylated, and then may interact with the MTBD through direct electrostatic interactions. Since the HEK293 cell line Applicants used only expresses the 4R MTBD repeats of tau (K18 fragment), the polyserine is interacting with these repeats, either directly or indirectly. Applicants have also observed that 42-polyserine is recruited to full length ON4R tau expressed in H4 neuroglioma cells (FIG. 7D). This is relevant to diseases contexts as the 4R MTBD has been shown to be involved in forming the core of tau fibrils in AD, CTE, and CBD. This suggests that while endogenous SRRM2 is recruited to 62% of cytoplasmic tau aggregates, the C-terminus of SRRM2 or 42-serine repeats alone show recruitment to >90% of cytoplasmic tau aggregates (FIG. 5D). Notably, in one embodiment, polyserine may interact with monomeric tau or with aggregated tau. Such integration may be directly, or indirect through a tau-mediator.

The C-terminal region of SRRM2 and polyserine are sufficient to trigger a self-assembly reaction similar to a liquid-liquid phase separation (LLPS), and that may recruit tau proteins, which enhances its rate of fibrillization. This is argued by several observations. First, predictions of LLPS probability suggest that the c-terminus of SRRM2 (aa 2606-2752) scores highly. Second, the fact that the C-terminal domain of SRRM2 and 42-polyserine are sufficient both to induce MIG like assemblies (cytoplasmic speckles) and interact with tau aggregates suggest that a self-assembly property of SRRM2 is part of the mechanism (FIGS. 7E,F). The self-assembly of polyserine motifs might be related to their length dependent tendency to promote the formation of coiled-coils.

Additional proteins also localize to tau aggregates via similar mechanisms that could be similar to SRRM2. The present inventors identified two other nuclear proteins, PNN and SETDIA, that are enriched into tau aggregates and contain long (>20 a.a.) polyserine repeats (FIGS. 5E,F). Additionally, the polyserine rich C-terminus of PNN is also sufficient to localize a halo fusion protein to tau aggregates (FIG. 7C). PNN present at ˜1.5 million molecules/cell and is the most abundant polyserine containing protein Applicants have identified. In agreement with the idea that polyserine is important for tau aggregation, knockdown of PNN using three different siRNAs reduced the percentage of tau aggregate positive biosensor cells and the integrated FRET density of cells by as much as 34% and 62% respectively (FIGS. 7A,B).

In one embodiment, polyserine repeats could also be used to create novel therapeutics or imaging reagents that target tau aggregates. For example, polyserine could be fused to phosphatases, ubiquitin ligases, ubiquitin segregases, fluorophores, helicases, RNases, etc. to locally influence the microenvironment in and around tau aggregates. Nuclear RNA and RNA binding proteins have been observed to be present in tau aggregates in model systems and patient post-mortem samples. This work describes how the nuclear splicing factor, SRRM2 is recruited to tau aggregates and implicates the cell cycle and polyserine repeats as key factors in this interaction. More broadly, the present invention may help explain RNA processing defects seen in tauopathies, identify other tau aggregate interacting proteins, and suggest ways to develop novel therapeutics for tauopathies.

### Example 2: Tau Aggregates can Occur in Conjunction With Cytoplasmic SRRM2 Assemblies or Form Independently

In principle, SRRM2 could mis-localize to cytoplasmic tau aggregates by two different mechanisms. Tau aggregates could form independently of SRRM2, and the shuttling of SRRM2 between the nucleus and cytoplasm could lead to SRRM2 becoming trapped in cytoplasmic tau aggregates. Alternatively, since SRRM2 can form cytoplasmic assemblies, referred to as mitotic interchromatin granules (MIGs), cytoplasmic SRRM2 assemblies could serve as nucleation sites for tau aggregation and/or merge with existing cytoplasmic tau aggregates. MIGs are thought to form during mitosis, but MIG-like assemblies, which are generally referred to as ‘cytoplasmic speckles,’ can also occur when the cytoplasmic concentration of SRRM2 is increased in non-mitotic cells. Moreover, SRRM2+ cytoplasmic speckles are detected in amyloid-β models of mice and in human postmortem tissue. Consistent with these earlier results, the present inventors observed SRRM2+cytoplasmic speckles in HEK293 cells both during mitosis, and in a fraction of non-mitotic cells (FIG. 1E). To further examine how SRRM2 co-localizes with cytoplasmic tau aggregates, Applicants examined HEK293 tau biosensor cells expressing an SRRM2-Halo fusion protein over time following seeding with tau aggregates from P301S mouse brain extracts.

Strikingly, this experiment demonstrated two conditions where tau fibrillization can occur on the surface of cytoplasmic SRRM2 assemblies. First, Applicants observed that SRRM2 can shuttle to the cytoplasm independent of mitosis and form cytoplasmic SRRM2+ assemblies. Tau aggregation can be initiated from these mitosis independent cytoplasmic SRRM2 assemblies and proceed to grow (FIG. 1A) and previous results have shown that amyloid-β toxicity can induce cytoplasmic SRRM2 aggregates in mouse and human neurons. In a second mechanism, Applicants also observed the formation of SRRM2+ MIGs during mitosis, which also served as sites of tau propagation, or merged with cytoplasmic tau aggregates (FIG. 1B). This identifies MIGs, or SRRM2+ cytoplasmic speckles, as subcellular sites with an increased rate of tau seed propagation, with this process occurring in 47.4% of the cases of tau aggregation (FIG. 1D). This demonstrates in cells that a specific subcellular location can be a preferred site of protein fiber propagation.

In a third mechanism, which occurred in 52.6% of the time (FIG. 1D), tau aggregates formed in the cytoplasm independently of detectable cytoplasmic SRRM2 and then a rapid (<10 minute) bulk movement of SRRM2 from the nucleus to the cytoplasm merged SRRM2 with the growing tau aggregate (FIG. 1C), which Applicants refer to as nuclear collapse. Following such nuclear collapse, Applicants did not observe SRRM2 return to the nucleus during the time span of the recording (48 hours). Analysis of the prevalence of these three mechanisms for SRRM2relocalization to cytoplasmic tau aggregates shows that nuclear collapse is the most common (52.6%), then MIG nucleation (31.6%), followed by mitosis-independent formation of SRRM2+ cytoplasmic speckles (15.8%) (FIG. 1D).

Taken together, these results suggest the following: First, tau aggregates can nucleate from and merge with SRRM2+ cytoplasmic speckles. Second, there are multiple modes by which tau aggregation can propagate and provides an explanation for the fact that only some tau aggregates contain substantial SRRM2. This suggests that SRRM2+ cytoplasmic speckles create an environment that is sufficient for the nucleation and growth of tau aggregates.

### Example 3. The C Terminal Region of SRRM2 is Necessary and Sufficient for Interactions With tau Aggregates

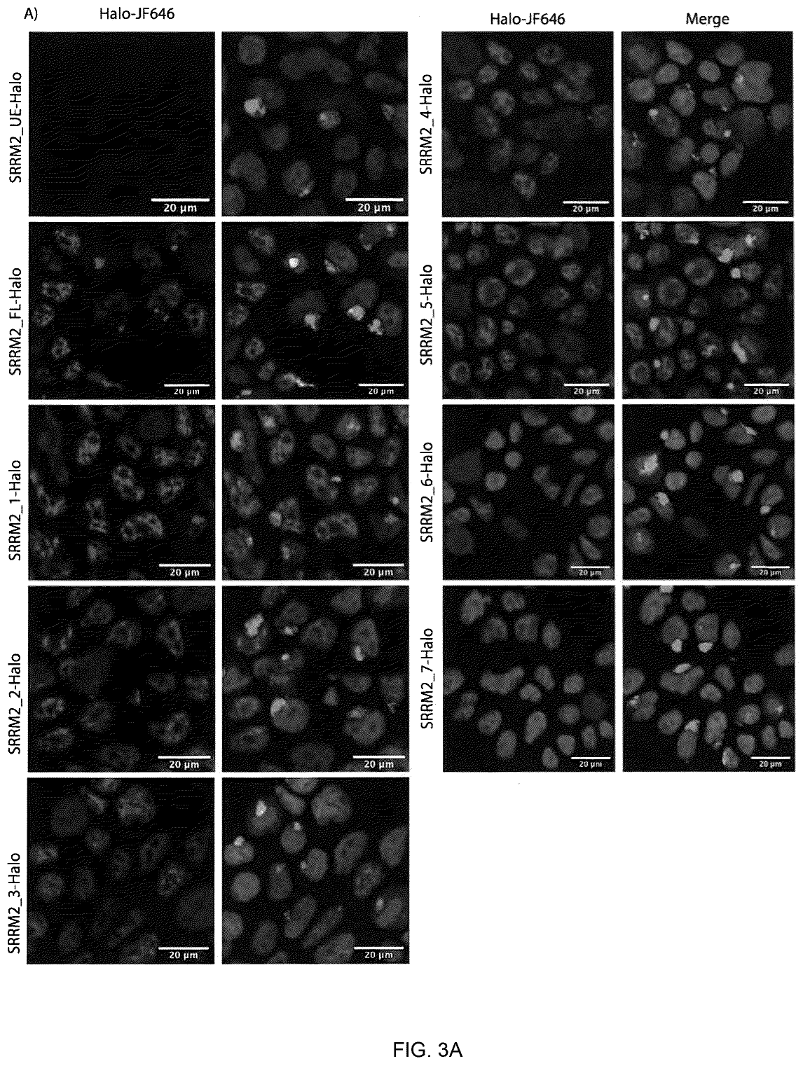

It had been previously suggested that the long C-terminal disordered region of SRRM2 was required for its mis-localization to cytoplasmic tau aggregates. To determine if there is specific domain in SRRM2's disordered region that dictates its mis-localization to cytoplasmic tau aggregates, the present inventors constructed a series of truncated SRRM2 proteins by inserting Halo tags into chromosomal copies of the SRRM2 gene using the CRISPaint system. All constructs were expressed as well using the endogenous promoter and ran at the appropriate size on SDS PAGE (FIGS. 2A, B). The multiple bands observed in truncations 5-7 could represent unannotated splice isoforms of SRRM2 or post-translational modifications, which are revealed because of the smaller size of these truncation proteins (FIG. 2B). Applicants then examined whether these truncations were recruited to tau aggregates formed by seeding in the HEK293 tau biosensor cells Analysis of these deletions demonstrated that the last 294 amino acids in the C terminal region of SRRM2 are necessary for recruitment to tau aggregates. Specifically, Applicants observed that deletion of the last 146 amino acids (SRRM2_1, a.a. 2606-2752) reduced the recruitment of SRRM2 into tau aggregates from a mean enrichment of 2.57 in the full length SRRM2 to 1.67 in the truncated form. A larger 294 amino acid deletion (SRRM2_2, a.a. 2458-2752) even further reduced the co-localization of SRRM2 in tau aggregates to a mean enrichment of 1.26. (FIGS. 2C-E). All larger deletions failed to co-localize with tau aggregates (FIGS. 2D, 3). This suggests that there are two elements within the C-terminal region of SRRM2 that can influence its recruitment to tau aggregates.

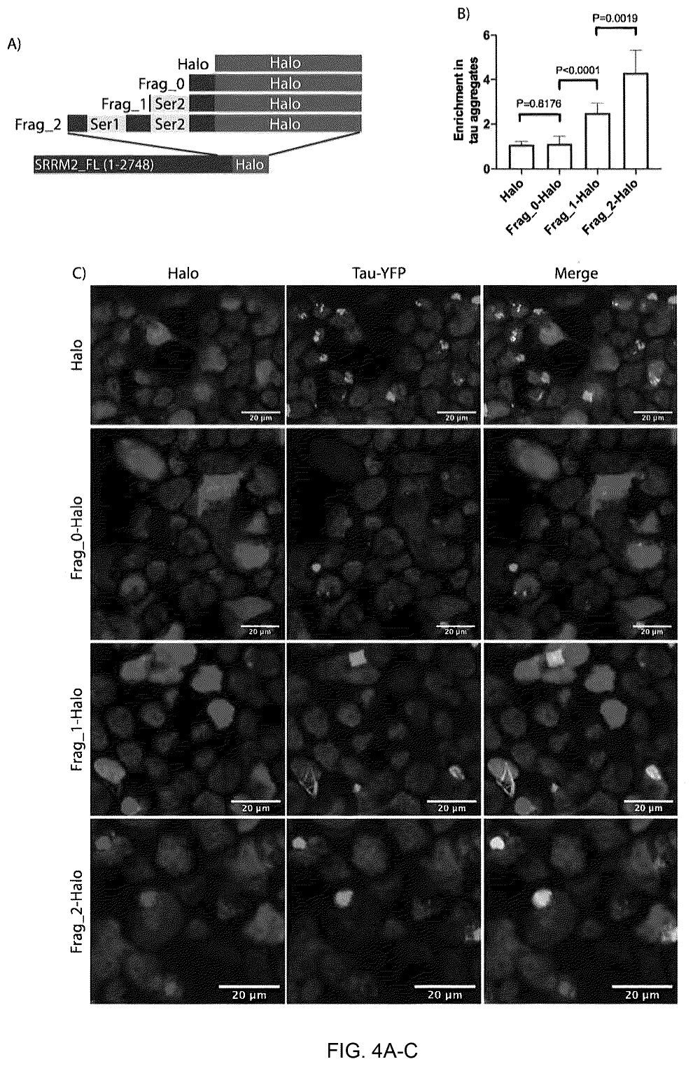

This analysis also maps the C-terminus as being required for accumulation of SRRM2 in cytoplasmic speckles. Specifically, truncation of the last 294 amino acids from the C terminus reduced the accumulation of SRRM2 in MIGs from 0.15 MIGs per nuclei to 0.05 MIGs per nuclei (FIGS. 2F, G). This demonstrates that the same region of SRRM2 required for interaction with tau is also required for accumulation in MIGs. To test whether expression of the C-terminal region of SRRM2 was also sufficient to localize to tau aggregates, Applicants expressed halo tagged C-terminal fragments using plasmids (FIG. 4A). Applicants observed that Frag-2-Halo (a.a. 2458-2752) accumulated robustly in cytoplasmic tau aggregates (mean enrichment of 4.3), while Frag_1-Halo (a.a. 2606-2752) accumulated in tau aggregates to a lesser extent (mean enrichment of 2.50) (FIGS. 4B, C). Applicants observed that neither Frag_0-Halo (a.a. 2651-2752) nor Halo alone accumulated in tau aggregates (mean enrichment of 1.1 and 1.08 respectively) (FIGS. 4B-C). This observation is consistent with the deletion results and demonstrates that there are two elements in the last 294 a.a. (a.a. 2458-2752) of SRRM2's c-terminus mediating its interaction with tau aggregates.

Taken together, these observations demonstrate the C-terminal region of SRRM2 is necessary for accumulation in tau aggregates and can form MIG-like assemblies.

### Example 4. Polyserine is Necessary and Sufficient for Localization to tau Aggregates

The observations above suggested there were two elements within the C-terminal region of SRRM2 that could contribute to its recruitment to cytoplasmic tau aggregates. Analysis of the amino acid sequence in the C-terminus of SRRM2 showed two long polyserine stretches in these regions (Ser1 repeat is 42 serines long and Ser2 repeat is 25 serines long) (FIG. 4A). Considering this, Applicants found that the number of polyserine repeats in the fragments Applicants expressed correlated with their recruitment into tau aggregates (FIG. 4B). To determine whether polyserine by itself was sufficient for localization to tau aggregates, Applicants cloned halo tagged polyserine repeats of varying lengths (5, 10, 20, 42) into a mammalian expression plasmid and measured their enrichment in tau aggregates. All constructs were expressed well and ran at the appropriate size (FIGS. 5A-B).

A key observation was that a region containing 42 consecutive serines was sufficient to robustly target the Halo tag to tau aggregates (mean enrichment of 5.46). Applicants found a slight enrichment with 20-serines (mean enrichment of 1.19), and little to no enrichment with the 5 or 10-serines (mean enrichment of 0.95 and 1.05 respectively) (FIGS. 5A, C). Thus, polyserine is sufficient to specify interaction of proteins with tau aggregates.

Applicants also observed that the 42-polyserine tract was sufficient to target the halo tag to either cytoplasmic or nuclear tau aggregates (FIG. 5A, white arrows). In the absence of tau aggregates, all of the polyserine constructs diffusely localized throughout the nucleus and cytoplasm, which is consistent with previous observations of diffuse 30-serine-YFP localization in COS-7 cells (Oma et al., 2004). This suggests that polyserine repeats are sufficient for localization to either nuclear or cytoplasmic tau aggregates and this localization becomes more robust with longer repeat lengths.

Previous observations have shown that not all cytoplasmic tau aggregates in HEK293 and H4 tau biosensor cells contain SRRM2. One possible explanation for this heterogeneity is that a variety of tau conformers are created in response to seeding from mouse brain homogenate and the polyserine regions in SRRM2 differentially interact with these various conformers. Another possibility is that polyserine can interact with all tau conformer types and SRRM2's ability to mis-localize to tau aggregates requires a second event, such as re-localization to the cytosol. To address this, the present inventors compared the percentage of cytoplasmic tau aggregates that were positive for SRRM2_FL-Halo, Frag_2-Halo, and 42-Serine-Halo (FIG. 5D). Applicants found that 42-serine-Halo and the Frag_2-Halo C-terminal fragment were more enriched in cytoplasmic tau aggregates than full length SRRM2-halo (96%, 98%, and 62% respectively). This suggests that the heterogeneous nature of SRRM2's recruitment to cytoplasmic tau aggregates is likely due to regulation of SRRM2's nuclear-cytoplasmic transit by an element in the N-terminus or middle of the protein rather than the ability of the polyserine repeats in the C-terminus to interact with tau aggregates. This is consistent with the observation that SRRM2 is recruited to tau aggregates in AD and CBD, two tauopathies that have been shown to have different tau fibril conformations by cryo-electron microscopy.

Two observations highlight the generality of the ability of polyserine to target exogenous proteins to tau aggregates. First, polyserine tracts are also sufficient to target exogenous proteins to tau aggregates in H4 cells expressing full-length ON4R tau (FIG. 7D). This demonstrates the interaction of polyserine with tau aggregates is not limited to those formed from just the K18 repeat domains of tau (expressed in the HEK293 tau biosensor cells). Second, Applicants observed that two proteins that contain stretches of greater than 20 consecutive polyserine, PNN and SETDIA, both localize to tau aggregates (FIGS. 5E,F). Moreover, the polyserine region of PNN, which is a 67 amino acid region in the c-terminus where 75% of the amino acids are serines, is sufficient to target exogenous proteins to tau aggregates (FIG. 7C).

Our data demonstrate that polyserine is necessary and sufficient to target SRRM2, to target tau aggregates in a variety of contexts, including when coupled with a fusion peptide having an exogenous, or heterologous peptide, the terms being generally used interchangeably herein. This provides a molecular explanation for how SRRM2 accumulates in tau aggregates in cell lines, mouse neurons, and in post-mortem tissues from multiple different tauopathies.

### Example 5. Cytoplasmic Assemblies of Polyserine or Srrm2-ct are Conducive to tau Propagation

Two observations led the present inventors to hypothesize that polyserine might form assemblies sufficient to enhance the rate of tau fiber propagation. First, polyserine is sufficient to interact with tau aggregates. Moreover, cells with overexpressed 42-polyserine-Halo or SRRM2-ct-Halo fusion proteins formed cytoplasmic assemblies (FIGS. 7E,F). If polyserine is sufficient to create a biochemical environment conducive to tau fiber propagation, one predicts that these artificially induced polyserine-halo or SRRM2-ct-halo assemblies should serve as sites of tau propagation. To examine this possibility, Applicants expressed Ser42-Halo or SRRM2-ct-Halo in tau biosensor lines, seeded those cells with brain extracts from P301L mice, and continuously imaged the cells (FIGS. 6A-D).

A striking result was that Applicants observed tau aggregates preferentially initiated on the surface of both SRRM2 c-terminal and 42-polyserine assemblies, similar to what Applicants observed with full length SRRM2-halo (FIGS. 6A-D). Out of 50 tau aggregates in cells expressing SRRM2 C-terminal-halo, 62% of tau aggregates initiated from SRRM2-C-terminus-halo assemblies and in cells expressing 42-polyserine-halo, 78% of tau aggregates initiated from 42-polyserine-halo assemblies (FIGS. 6B, C). This implies that polyserine by itself is sufficient to create a local environment conducive to tau propagation either by direct interactions with tau, or through the recruitment of an intermediate protein or RNA.

### Example 6. Knockdown of the Abundant Polyserine Contain Protein, PNN, Decreases tau Aggregation

The above results suggest a model whereby the surface of cytoplasmic assemblies enriched in polyserine domains can serve as a discrete biochemical environment conducive to propagation of tau fibers. A prediction of this model is that decreasing the amount of cytoplasmic assemblies containing polyserine domains would correspondingly decrease the rate of tau fiber propagation. Although it is unclear what the rate limiting step in tau fiber propagation is in any biological context, Applicants have addressed this issue in two manners.

First, Applicants knocked down the two most abundant proteins containing polyserine tracks associated with MIGs, SRRM2 (present at ˜500,000 molecules/cell) and its binding partner PNN (present at ˜1.5 million molecules/cell). Applicants targeted these two proteins for analysis since they both accumulate in tau aggregates and contain polyserine domains sufficient to target exogenous proteins to tau aggregates. Applicants used three siRNAs targeting PNN and observed that PNN knockdown reduced the percentage of tau aggregate positive biosensor cells and the integrated FRET density of cells by as much as 34% and 62% respectively (FIGS. 7A, B). SRRM2 knockdown appeared toxic to cells by microscopic examination. Interestingly, PNN knockdown had a similar effect on tau aggregates as knockdown of MSUT2 (mammalian suppressor of tauopathy), a protein shown to alter tau aggregation in mouse and invertebrate model organisms (FIGS. 7A,B). Taken together, these results indicate that altering the levels of proteins with polyserine domains can impact the amount of tau aggregates.

### Example 7. Targeting tau Aggregates Using Polyserine Fusion Proteins

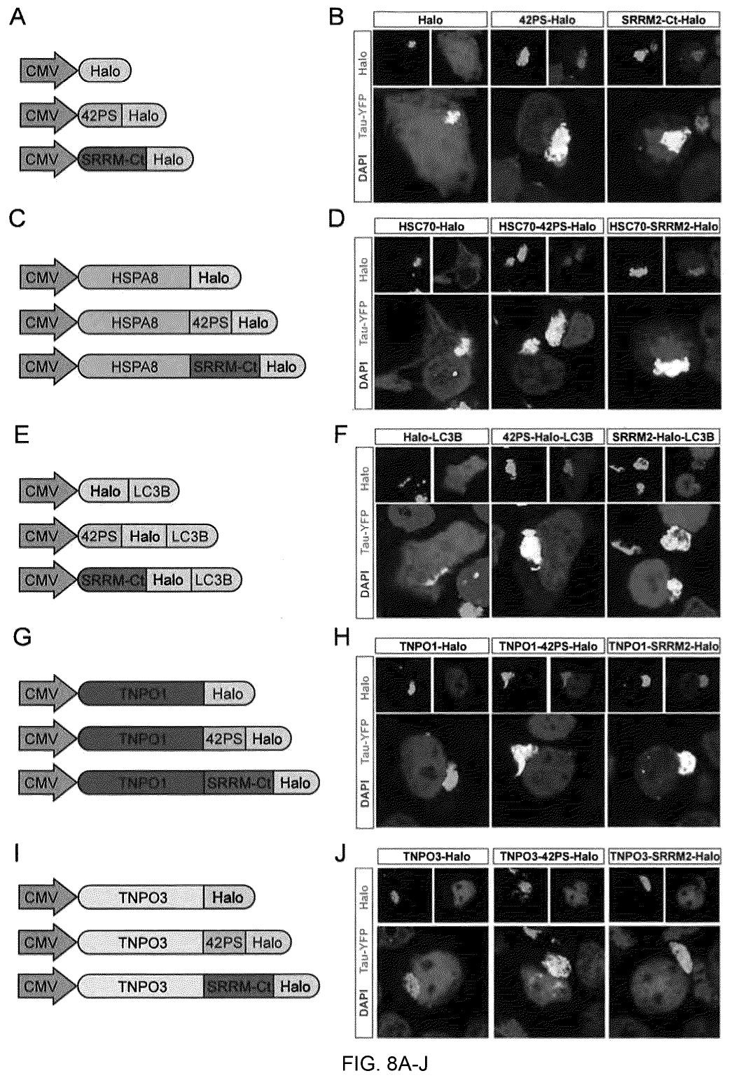

To determine whether the tau targeting moieties of the invention could alter tau aggregation in a model system, Applicants fused 42-polyserine and SRRM2 C-terminus to HSPA8 (HSC70), LC3B, TNPO1, TNPO3, and VCP (all tagged with HaloTag® (SEQ ID NO. 47) (FIG. 8). Applicants then expressed the TNPO3 and VCP fusion proteins in HEK293 tau biosensor cells along with appropriate controls (no targeting moiety (Halo), 42PS-Halo no insert, and SRRM2 C-terminus no insert). Applicants measured the amount of tau aggregation in the cells using a previously described flow cytometry assay via two methods: 1) FRET+ cells (normalized to no insert Halo) and 2) Integrated FRET density (normalized to no insert Halo). Applicants found that targeting both valosin-containing protein (VCP) (SEQ ID NO. 38) and transportin-3 (TNPO3) (SEQ ID NO. 39), to tau aggregates using either 42PS or SRRM2 C-terminus resulted in a roughly 50% decrease in tau aggregation by both measurement strategies. This experiment shows that targeting active fusion proteins to tau aggregates using polyserine motifs reliably alters tau aggregation in cells.

Exemplary fusion peptides generated herein include: 42-polyserine-VCP-halo (SEQ ID NO. 30, 31); 42-polyserine-TNPO3-halo (SEQ ID NO. 32, 33); SRRM2 C-terminus-VCP-halo (SEQ ID NO. 34, 35); SRRM2 C-terminus-TNPO3-halo (SEQ ID NO. 36, 37)

### Example 8: Polyserine is Sufficient to Target Exogenous Proteins to tau Aggregates

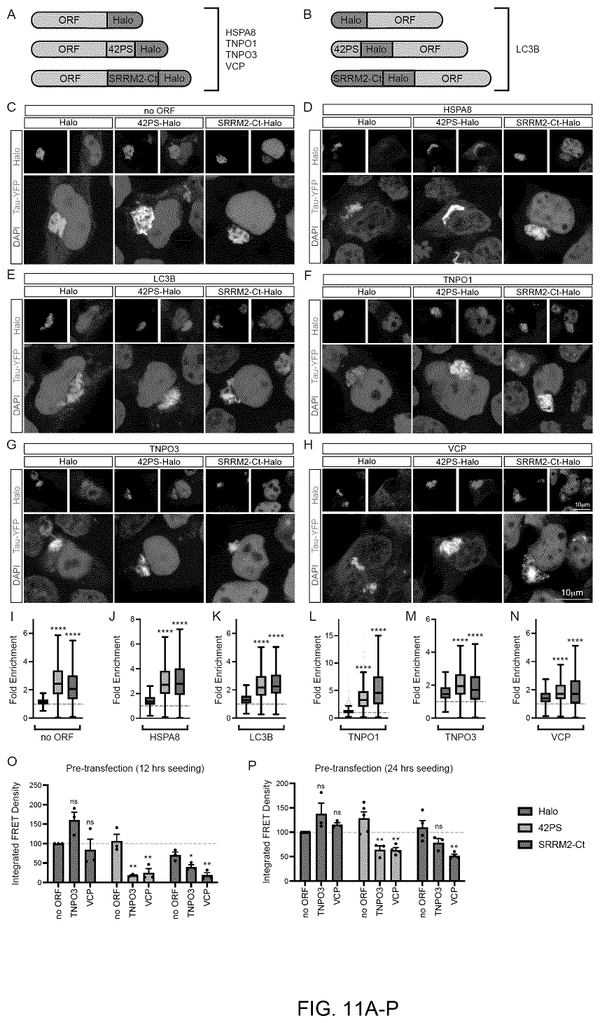

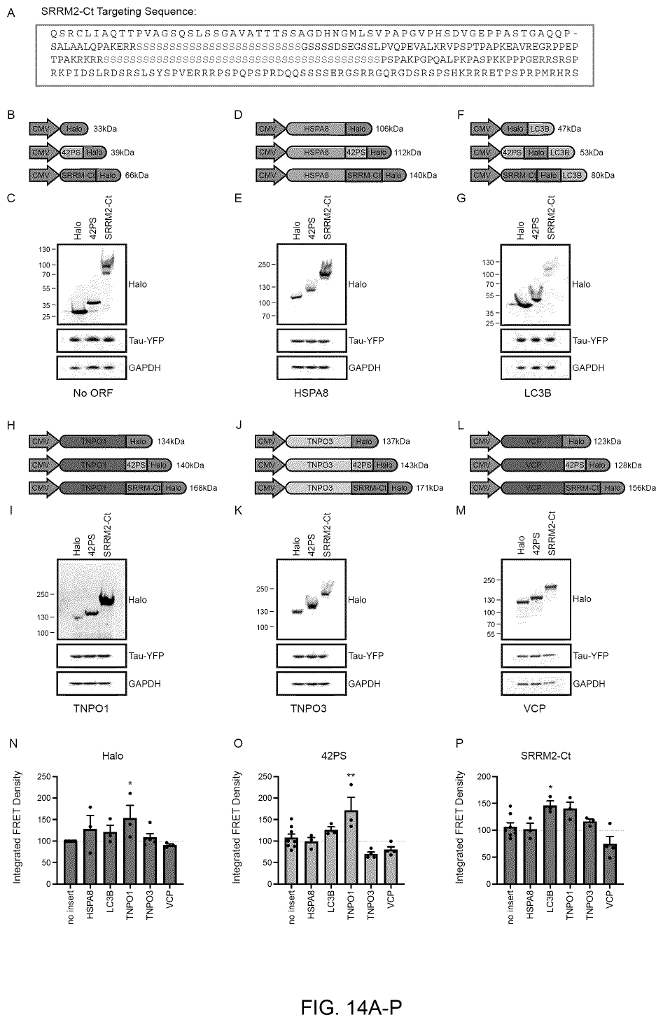

As previously shown, polyserine motifs are the defining feature mediating the mislocalization of the nuclear speckle proteins SRRM2 and PNN to tau aggregates (16). Furthermore, polyserine stretches are sufficient to target a Halo reporter protein to tau aggregates in a length dependent manner (16). For polyserine to be useful as a targeting module to disrupt tau aggregation it would be beneficial to effectively target a range of proteins to tau aggregates. To determine the breadth of utility for polyserine-based targeting, Applicants generated a range of Halo-tagged fusion proteins with or without a polyserine based targeting motif (FIGS. 11A,B (see below)). These targeting motifs consisted of a stretch of 42-serines, or a C-terminal fragment of SRRM2 which contains two polyserine stretches of 25 and 42-serines (FIG. 14A).

Fusion proteins were generated with candidate ORFs for modulation of tau aggregation and expressed in HEK293 tau biosensor cells, which express the tau microtubule repeat domain region fused to CFP or YFP. In this initial set of experiments, Applicants constructed fusions to HSPA8—an Hsp70 family chaperone which has been shown to possess tau disaggregation properties in coordination with J-domain proteins in vitro (14,17), LC3B-an autophagy factor involved in autophagosome formation (18), the nuclear import receptors TNPO1 and TNPO3 that might limit tau aggregation analogous to their activity towards FUS and TDP-43 (6,7,9), and VCP—a segregase which has been shown to disassemble tau aggregates (12,13). Expression of these fusion proteins and a lack of changes in tau levels was validated by Western blot (FIGS. 14B-M). Applicants observed that addition of a polyserine domain was sufficient to increase recruitment of all the fusion proteins to tau aggregates. Specifically, Applicants quantified the fold enrichment of Halo-tagged proteins in tau aggregates induced by lipofection of clarified brain homogenate from tau transgenic mice (Tg2541) mice. Applicants observed a significant increase in all tested polyserine containing proteins (FIGS. 11C-N). Apart from LC3B, fusion proteins were generated with a C-terminal polyserine motif. As LC3B is subject to post-translational cleavage and lipidation at the C-terminus the polyserine motif and Halotag were added to the N-terminus. Therefore, polyserine is sufficient to target a range of exogenous proteins to the local environment of tau aggregates irrespective of the location of the motif in the fusion protein.

### Example 9: Targeting of Fusion Proteins Augments Modulatory Effects on tau Aggregation

To assess whether overexpression of Halo-tagged proteins in targeted or nontargeted forms modulate tau aggregation, Applicants performed flow cytometry utilizing FRET as a measure of tau aggregation with an additional sorting step for Halo expressing cells. As previously reported, Applicants observe that overexpression of polyserine leads to a modest increase in tau aggregation (FIG. 11A) (16).

Applicants observed that fusion proteins could affect tau aggregation in three manners. First, Applicants observed that LC3B or HSP8A fusion proteins did not lead to significant alteration in tau aggregation (FIGS. 14 N, O, P). Second, Applicants observed that overexpression of TNPO1 led to increases in tau aggregation with or without polyserine (FIGS. 14 N, O, P). Third, Applicants observed that overexpression of TNPO3 and VCP do not modulate tau aggregation when not targeted to tau aggregates, but when fused to polyserine motifs lead to significant reductions in tau aggregation. This effect was most obvious 12 hours after seeding and declined with at 24 hours, but was still significant (FIGS. 11O & P). Similar results were observed if the C-terminal 292 amino acid domain of SRRM2, which is also sufficient to target proteins to tau aggregates, was used (FIGS. 11O&P) (16). The reduction in tau aggregation was observed without a change in tau levels (FIG. 14). Taken together, these findings indicate that the addition of polyserine targeting motifs can facilitate or enhance modulatory roles for exogenously expressed proteins on tau aggregation.

### Example 10: The VCP Adaptor Protein UBXD8/FAF2 Uniquely Reduces tau Aggregation

VCP is a segregase that interacts with ubiquinated proteins and utilizes ATP hydrolysis to extract proteins from those complexes (19). VCP functions in a wide variety of contexts and utilizes specific adaptor proteins to couple VCP to the ubiquinated targets (19). As VCP is a very abundant protein, Applicants hypothesized that targeting a VCP adaptor to tau aggregates with polyserine might limit tau aggregation to a greater extent. Given this, Applicants fused a set of VCP adaptor proteins to polyserine and examined their effect on tau aggregation. Specifically, Applicants generated Halo and 42PS-Halo tagged forms of three VCP adaptor proteins UBXD2, UBXD7 and Fas associated factor family member 2 (FAF2) (also known as UBXD8 and sometimes referred to as UBXD8/FAF2) (SEQ ID NO. 44). Applicants performed flow cytometry following transfection of targeted and non-targeted constructs and seeding of tau aggregates with tau brain homogenate in biosensor cells.

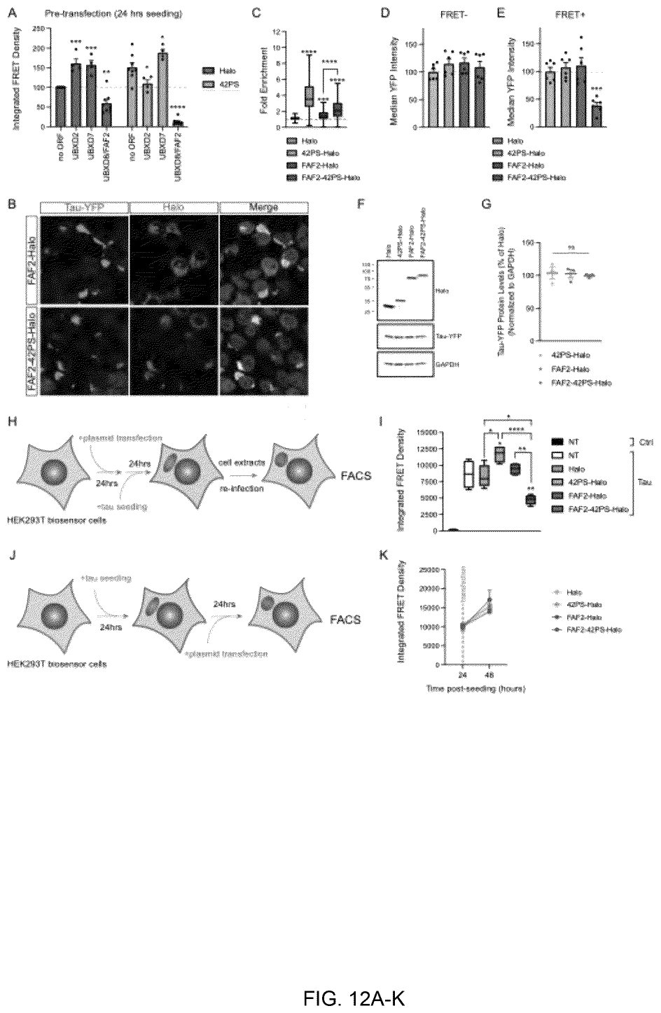

Importantly, Applicants observed a reduction in tau aggregation in samples with targeted FAF2 overexpression. This inhibition of tau aggregation became more pronounced when Applicants add a gating step to include transfected (Halo+) cells and is even further enhanced when filtering for the 10% of cells with the highest Halo expression. Specifically, Applicants observed a modest suppression of tau aggregation with overexpression of untargeted FAF2, while targeting with polyserine leads to ˜90% reduction in tau aggregation (FIG. 12A). Consistent with the other fusion proteins, the addition of a polyserine motif to FAF2 led to an increase in the fold enrichment in tau aggregates quantified by immunofluorescence (FIGS. 12B,C). Thus, exogenous expression of FAF2 prevents tau aggregation, and this is most effective when targeted to tau aggregates. The higher effectiveness of FAF2 compared to other VCP adaptors suggest that FAF2 has specific properties that allow effective limitation of tau aggregation (see below). In principle, FAF2 could affect aggregation by simply reducing the amount of tau or by disassembling tau aggregates and specifically reducing tau protein in aggregates. Applicants observed that FAF2 overexpression does not alter total tau levels in unseeded biosensor cells as analyzed by Western blot (FIGS. 12F,G), suggesting that polyserine-FAF2 is not just reducing total tau protein levels. In line with this finding, the median YFP signal—a measure of tau levels—in the top 10% of Halo expressing cells following seeding but without aggregates (FRET−) is not significantly altered with expression of FAF2 in targeted or non-targeted forms (FIG. 12D). This indicates that polyserine-FAF2 does not generally reduce tau protein levels.

To determine if polyserine-FAF2 could promote the degradation of tau in aggregates, Applicants quantified the levels of tau in cells transformed with polyserine-FAF2 and containing tau aggregates. Applicants hypothesis was that if polyserine-FAF2 interaction with tau aggregates enhanced the degradation of tau, this would only be observed in cells with tau aggregates. Interestingly, Applicants observed a significant reduction in YFP median intensity in the population of cells with aggregates (FRET+) (FIG. 12E). This suggests that when targeted to tau aggregates by polyserine, FAF2 might enhance the degradation of tau proteins.

Collectively, this data identifies FAF2 as a VCP adaptor with the unique capability of preventing tau aggregation, and reducing tau levels in cells with aggregated tau species with polyserine-mediated targeting enhancing these effects.

### Example 11: Expression of FAF2 Suppresses tau Aggregate Formation Without Increasing Seeding Capacity

In principle, FAF2 could reduce accumulation of large tau aggregates assessed by flow cytometry, but potentially lead to increased seeding competent species not detected by FRET. To evaluate this possibility, Applicants transfected HEK biosensor cells with targeted and untargeted forms of FAF2 alongside negative controls, seeded to form tau aggregates, then obtained cell extracts from this population to utilize for reinfection into biosensor cells (FIG. 12H). Applicants prepared cell lysates from HEK tau biosensor cells which were transfected 24-hours prior to tau seeding for 24-hours and lipofected lug of each extract once again into HEK tau biosensor cells. Utilizing FRET-based flow cytometry 24 hours post re-infection, Applicants observed that cell extracts from cells transfected with 42PS showed a modest increase in seeding capacity-consistent with our prior findings that 42PS overexpression increased tau aggregation (FIG. 12I). Applicants did not observe a change in seeding competency in extracts from cells previously transfected with untargeted FAF2 overexpression relative to controls suggesting untargeted FAF2 does not alter new seed production. In contrast, cells with prior FAF2-42PS expression had significantly reduced seeding capacity (FIG. 12I). This data supports a model where expression of targeted FAF2 prior to tau seeding reduces tau aggregation without redirection of aggregate-prone tau species into smaller seeding competent species.

To assess whether FAF2 expression could also reverse existing tau aggregates, Applicants performed flow cytometry with biosensor cells that were first seeded to form aggregates, and subsequently transfected with Halo-tagged constructs. In this instance, transfection of targeted or untargeted FAF2 24 hours post-seeding did not lead to a reversal or slowing of tau aggregation relative to controls (FIGS. 12J,K). Thus, FAF2 overexpression is not sufficient to reverse or slow tau aggregation when expressed after significant tau aggregation has been established.

### Example 12: FAF2/UBXD8 Mitigates tau Aggregation Through Ubiquitin Recognition, Membrane Localization and VCP-Binding Domains

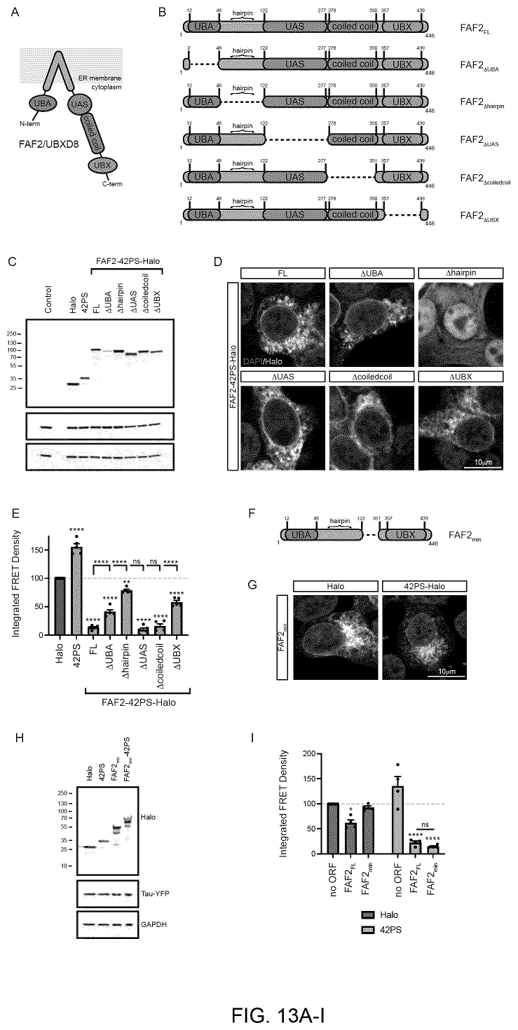

FAF2 is a cytosolic facing monotopic membrane protein that has been shown to localize to the ER—as well as lipid droplets and mitochondria—where it serves as a VCP adaptor (20,21) (FIG. 13A). At the ER, FAF2 complexes with additional factors involved in ER-associated degradation of terminally misfolded membrane and secretory proteins through VCP and proteasomal degradation (22).

To elucidate mechanistic insight into the required functions of FAF2 for suppression of tau aggregation, Applicants generated a series of deletion mutants fused to polyserine and Halo (FIG. 13B). Expression of constructs were validated by Western blot and localization monitored by immunofluorescence (FIGS. 13C,D). As anticipated, removal of the membrane hairpin domain resulted in a shift from an ER-like localization to diffuse signal throughout the cytoplasm whereas all other deletions displayed a comparable subcellular distribution to the full-length protein.

Applicants evaluated the activity of each FAF2 deletion mutant on reducing tau aggregation by flow cytometry. Applicants found deletion of the ubiquitin-associated (UBA), hairpin, and ubiquitin regulator X (UBX) domains led to an impaired suppression of tau aggregation (FIG. 13E). UBA domains facilitate ubiquitin binding while UBX domains adopt ubiquitin-like folds which serve as VCP binding sites (23,24). In contrast, the UAS and coiled-coil domains were dispensable for FAF2 activity in mitigating tau aggregation.

To assess which domains are minimally required Applicants designed a construct with only the UBA, hairpin, and UBX domains referred to as “FAF2min” (FIG. 13F). Applicants first validated constructs expressing a Halo tagged or 42 polyserine and Halo tagged version of FAF2min by Western blot and immunofluorescence and observed similar localization with both constructs as with full-length FAF2 (FIGS. 13G,H). Applicants next tested whether FAF2min maintained suppressive activity without polyserine-mediated targeting and observed that untargeted FAF2min lost the modest effect on limiting tau aggregation indicating effects of overexpressed FAF2 could involve the UAS and coiled-coil domains or it may affect association with tau aggregates (FIG. 13I). In contrast, targeted FAF2min showed analogous efficacy to targeted FAF2FL. These results identify that the FAF2 UBA, hairpin, and UBX domains are alone sufficient for suppress activity towards tau aggregation when targeted with polyserine (FIG. 13I).

Collectively these findings demonstrate that the UBA, hairpin and UBX domains of FAF2are both necessary and sufficient for effects on tau aggregation underlining the key role for membrane localization, ubiquitin recognition, and VCP recruitment.

### Example 13: Broad Applicability of Polyserine tau Targeting Element

As shown above, Applicants results indicate that polyserine is an effective and broadly applicable tau targeting element that can be used to alter tau aggregation. Applicants previously showed that a minimal stretch of 20 serine residues can enrich Halo protein at tau aggregates, with more efficient targeting occurring with 42 serine residues (16). Here, Applicants extend these findings to show that polyserine-mediated enrichment of exogenous proteins at tau aggregates occurs regardless of orientation and with a wider range of proteins. These results provide proof-of-concept that polyserine is a modular targeting moiety that could be added to other cargo to facilitate enrichment at tau aggregates.

Through investigation of a range of polyserine fusion proteins, Applicants demonstrate that polyserine-targeting can augment the effects of candidate disassembly proteins. Specifically, Applicants observed that polyserine enhances suppressive effects on tau aggregation when fused to TNPO3, VCP or FAF2/UBXD8. This finding-in combination with the result that polyserine enriches proteins at tau aggregates-suggests that polyserine improves activity of fusion proteins by increasing the local concentration at tau aggregates. This demonstrates a general strategy for targeting desired proteins to tau aggregates that can be used both to identify new proteins capable of limiting tau aggregate formation, and even as potential therapeutics when delivered by viral transduction or protein delivery systems (25).

Applicants further previously reported overexpression of polyserine alone leads to a small increase in tau aggregates that can form associated with assemblies of exogenous polyserine or polyserine containing endogenous proteins (16). Applicants observed that polyserine fusions to TNPO3, VCP or FAF2/UBXD8 did not readily form self-assemblies as polyserine alone. Thus, when fused to an effective suppressor of tau aggregation, the potential role of polyserine alone in promoting tau aggregation does not appear to be a significant damper.

The most effective suppressor of tau aggregation Applicants identified is FAF2 (SEQ ID NO. 44), the activity of which is improved by addition of a polyserine-targeting element. Applicants also demonstrate this role is dependent on ubiquitin and VCP binding domains, suggesting this activity is mediated through VCP-in line with reported disaggregase activity of VCP towards tau (13). Consistently, FAF2 has previously implicated in the VCP dependent regulation of RNP granules by targeting HuR to facilitate release from mRNP complexes and G3BP1 promoting stress granule disassembly (26,27). Interestingly, Applicants' data indicates this function in limiting tau aggregation is not shared across VCP adaptors further emphasizing a unique role for FAF2 (also sometimes referred to as FAF2/UBXD8) in the dissolution of assemblies or aggregates. FAF2/UBXD8 localizes to the ER membrane, as well as mitochondria and lipid vesicles-and Applicants' data indicate the ability to dock at membranes is required for tau aggregate suppression (20,21). Applicants further note that anchoring the VCP complex at membranes may be required to exert sufficient mechanical force on substrate proteins or require other similarly localized co-factors (19).

When considering strategies to reduce tau aggregation, a key confounding factor is whether the disassembly of larger aggregated species produces more smaller, seeding competent tau forms. Applicants observe that expression of polyserine-fused FAF2/UBXD8 prior to tau aggregate formation can suppress aggregation in a manner which does not increase seeding competency. However, a recent study showed that VCP-mediated disassembly of tau aggregates causes an increase in seeds (13). It is possible that this distinction is reflective of the different burden of tau aggregation. Thus, when encountering a tau seed or smaller oligomeric tau species, a cell expressing polyserine-FAF2/UBXD8 may be able to recruit VCP for disaggregase activity and re-fold or degrade tau. In fact, Applicants observe that in a cell with higher tau aggregate burden the rates of FAF2/UBXD8-mediated disaggregation do not outcompete the rates of tau aggregation.

Collectively, Applicants identify polyserine as an effective targeting strategy-and FAF2/UBXD8 as a suppressor of tau aggregation. Applicants previously showed that the polyserine containing protein SRRM2 is mislocalized to tau aggregates in tau transgenic mice as well as in CBD and AD patient tissue indicating associations between polyserine and tau are present across models and in disease states (15,28). Future work investigating whether polyserine as a targeting strategy and the suppressive activity of targeted FAF2/UBXD8 are conserved in neuronal and animal models will provide key insight into the therapeutic potential of this approach.

### Example 14. Materials and Methods (Examples 1-7)

Growth of HEK293 tau biosensor cells and tau aggregate seeding. As previously described, HEK293 biosensor cells stably expressing the 4R RD of tau with the P301S mutation were purchased from ATCC (CRL-3275) (previously described in (Holmes et al., 2014)). Cells were seeded at 2.5×10₅ cells/mL in 500 uL of DMEM with 10% FBS (for serum starvation conditions, 10% FBS was omitted) and 0.2% penicillin-streptomycin antibiotics on PDL coated glass coverslips in a 24-well tissue culture treated plate (Corning 3526) and allowed to grow overnight in incubators set to 37° C. with 5% carbon dioxide. The next day, 7 ug of 1 mg/mL clarified P301L tau or WT tau mouse brain homogenate was mixed with 6 uL of Lipofectamine 2000 and brought up to 100 uL in PBS and allowed to sit at room temperature for 1.5 hours. The mixture was then added to 300 uL of DMEM without FBS or antibiotics and mixed by pipetting. 50 uL of this mixture was added to each well of a 24 well plate and allowed to incubate at 37° C. for 24 hours. Tau aggregate formation was monitored using a fluorescence microscope with a 488 nm filter.

Clarification of brain homogenate for tau aggregate seeding in HEK293 cells. As previously described (Lester et al., 2021), 10% brain homogenate from Tg2541 or WT mice was centrifuged at 500×g for 5 minutes, the supernatant was transferred to a new tube and centrifuged again at 1,000×g for 5 minutes. The supernatant was again transferred to a new tube and the protein concentration was measured using bicinchoninic acid assay (BCA), and diluted in DPBS to 1 mg/mL for transfection into HEK293 tau biosensor cells.

Live cell imaging of tau aggregate formation and SRRM2 relocalization. HEK293 tau biosensor cells were seeded in 24 well glass bottom plates with #1.5 cover glass at 2.5*105 cells/ml with or without 200 nM JF646-Halo ligand and allowed to grow overnight at 37° C. To counter stain the nuclei, Hoescht 33342 was added to the cell culture media and allowed to incubate for 15 minutes prior to imaging. For tan aggregate formation, cells were imaged on a Nikon Spinning Disc microscope every 30 minutes in the DAPI and GFP channels for 24 hours at 37° C. and 5% CO2. For analysis of SRRM2 relocalization during tau aggregation, cells were imaged on an Opera Phenix High Content imaging system where images in the Cy5, GFP, and DAPI channels were acquired every 10 minutes for 48 hours at 37°° C. and 5% CO2.

Immunofluorescence: As described above, cells were grown in 24 well plates on PDL coated coverslips, fixed for 10 minutes in 4% paraformaldehyde, washed 3× with PBS, and permeabilized with 0.1% Triton-X100 for 10 minutes, Cells were then washed with PBS 3×, blocked in 5% BSA for 30 minutes, followed by addition of primary antibodies at indicated concentration in 5% BSA and incubated overnight at 4 deg on a rotator. Cells were washed 3× with PBS and incubated with secondary antibody in 5% BSA for 30 minutes at room temperature on a rotator. Cells were then washed 3× with PBS and incubated in DAPI for 5 minutes at a final concentration of lug/mL in PBS. Cells were washed one more time with PBS and mounted on glass microscope slides using ProLong Glass Antifade Mountant.

CRISPaint tagging of SRRM2 in HEK293 tau biosensor cells. As previously described, HEK293 tau biosensor cells were seeded at 5*10\hat{ }5 cells/ml in 6 well plates and allowed to grow overnight in a 37° incubator. The next day, lipofectamine 3000 was used to transfect cells with 0.5 ug of sPsCas9 (BB)-2A-GFP (PX458) targeting plasmid (backbone is addgene #48138, sgRNA sequences used to target various truncations in SRRM2 can be found in Table 1), 0.5 ug of either 0, 1, or 2 pCAS9-mCherry-Frame selector plasmid (addgene #66939, 66940, 66941), and lug of pCRISPaint-HaloTag-PuroR plasmid (addgene #80960). Cells were allowed to grow for 24 hours and then 2 ug of puromycin was added to the culture media for 48 hours to select for cells that had incorporated the Halo-PuoR construct. JF646 was added to the media at a final concentration of 200 nM for 24 hours to covalently label the Halo fusion proteins for visualization by fluorescence imaging and analysis by gel electrophoresis.

TABLE 1
CRISPaint sgRNAs for halo tagging SRRM2

| | | | Amino |
|---|---|---|---|
| | | | acids |
| | | | lost |
| | | Frame | from |
| Name | Sequences | selector | c-term |
| SRRM2_FL_up | CACCGCCATGAGAC | 0 | 4 |
| | ACCGCTCCTCC | | |
| | (SEQ ID NO. 3) | | |
| SRRM2_FL_down | AAACGGAGGAGCGG | | |
| | TGTCTCATGGC | | |
| | (SEQ ID NO. 4) | | |
| SRRM2_1_up | CACCGCTAGAGCGC | 2 | 150 |
| | CTCTTCCGTT | | |
| | (SEQ ID NO. 5) | | |
| SRRM2_1_down | AAACAACGGAAGAG | | |
| | GCGCTCTAGC | | |
| | (SEQ ID NO. 6) | | |
| SRRM2_2_up | CACCGATTGGTCTG | 0 | 299 |
| | AAAAAGCAGA | | |
| | (SEQ ID NO. 7) | | |
| SRRM2_2_down | AAACTCTGCTTTTT | | |
| | CAGACCAATC | | |
| | (SEQ ID NO. 8) | | |
| SRRM2_3_up | CACCGCTTGCTGCA | 0 | 606 |
| | GGACAGACAT | | |
| | (SEQ ID NO. 9) | | |
| SRRM2_3_down | AAACATGTCTGTCC | | |
| | TGCAGCAAGC | | |
| | (SEQ ID NO. 10) | | |
| SRRM2_4_up | CACCGTTTCCAGCT | 0 | 1393 |
| | GATTAAGAAA | | |
| | (SEQ ID NO. 11) | | |
| SRRM2_4_down | AAACTTTCTTAATC | | |
| | AGCTGGAAAC | | |
| | (SEQ ID NO. 12) | | |
| SRRM2_5_up | CACCGGCGTTCTAG | 0 | 1947 |
| | ATTTAGCTTT | | |
| | (SEQ ID NO. 13) | | |
| SRRM2_5_down | AAACAAAGCTAAAT | | |
| | CTAGAACGCC | | |
| | (SEQ ID NO. 14) | | |
| SRRM2_6_up | CACCGGTCTAGGTC | 0 | 2194 |
| | AGCAAGGCGA | | |
| | (SEQ ID NO. 15) | | |
| SRRM2_6_down | AAACTCGCCTTGCT | | |
| | GACCTAGACC | | |
| | (SEQ ID NO. 16) | | |
| SRRM2_7_up | CACCGCTGGCATGC | 2 | 2322 |
| | CGAGAAACTT | | |
| | (SEQ ID NO. 17) | | |
| SRRM2_7_down | AAACAAGTTTCTCG | | |
| | GCATGCCAGC | | |
| | (SEQ ID NO. 18) | | |

Cloning and expression of SRRM2 C-terminal fragments and polyserine repeats. To express SRRM2 C-terminal fragments in a pcDNA-PuroR expression plasmid, RNA was extracted from SRRM2_FL-Halo cells using Trizol, and reverse transcribed to cDNA using oligodT primers and Superscript III. PCR primers containing 20 bp overhangs with a pcDNA plasmid digested with EcoRV and Xbal were used to amplify regions in the C-terminus of SRRM2_FL-Halo (See Table 2 for PCR primer sequences). PCR products were gel purified and cloned into EcoRV/Xbal digested pcDNA plasmid using In-Fusion cloning, transduced into Stb13 competent E. coli, and streaked out on LB ampicillin plates. Colonies were selected, grown in liquid culture, and mini-prepped for transfection into HEK293 cells. Similarly, for the polyserine-halo constructs, gene blocks were ordered from IDT with 20 bp overhangs and codons optimized for synthesis of polyserine repeats. These gene-blocks were then In-Fusion cloned into pcDNA plasmids and grown up for transfection.

TABLE 2
PCR primers Gel electrophoresis

| Primer name | Sequence |
|---|---|
| Infusion_SRRM2- | TTAAACGGGCCCTCT |
| cterm_halo_rev | AGTCTAGATCAACCG |
| | GAAATCTCCAGAGTA |
| | G |
| | (SEQ ID NO. 19) |
| Infusion_halo_ | GGTGGAATTCTGCAG |
| fwd | ATATCgccAccATGG |
| | AAATCGGTACTGGCT |
| | TTCC |
| | (SEQ ID NO. 20) |
| Infusion_SRRM2_ | GGTGGAATTCTGCAG |
| Frag_0_halo_fwd | ATATCgccAccATGA |
| | AGCCTGGCCCTCAGG |
| | CCTT |
| | (SEQ ID NO. 21) |
| Infusion_SRRM2- | GGTGGAATTCTGCAG |
| Frag_1_halo_fwd | ATATCGCCACCATGC |
| | GCTCTAGCAGTTCCA |
| | G |
| | (SEQ ID NO. 22) |
| Infusion_SRRM2- | GGTGGAATTCTGCAG |
| Frag_2_halo_fwd | ATATCGCCACCATGC |
| | AATCCCGTTGTTTGA |
| | TTGC |
| | (SEQ ID NO. 23) |

Cells in a 6 well plate were grown until 50-80% confluence, washed 1× with PBS, and trypsinized in 0.5 mL of trypsin. Cells were collected in a 1.5 mL microcentrifuge tube and centrifuged at 500 g for 5 minutes, washed 1× with PBS, and brought up in 100 uL of lysis buffer (25 mM Tris pH 7.5, 5% glycerol, 150 mM NaCl, 2.5 mM MgCl₂, 1% NP-40, 1:20 BME, 1X phosphatase/protease inhibitor). Lysate was pipetted up and down to mix and incubated on ice for 5 minutes. Lysate was then centrifuged at 16,000 g for 5 minutes and supernatant was transferred to a new tube and protein concentration was measured via Bradford. 10-15 ug of protein was combined with 4X LDS loading dye and boiled for 7 minutes prior to loading on a NuPAGE 4 to 12% Bis-Tris mini protein gel. Gels were either directly imaged if examining covalently linked JF646 or semi-dry transferred for western blotting.

Immunofluorescence. As described above, cells were grown in 24 well plates on PDL coated coverslips, fixed for 10 minutes in 4% paraformaldehyde, washed 3x with PBS, and permeabilized with 0.1% Triton-X100 for 10 minutes. Cells were then washed with PBS 3×, blocked in 5% BSA for 30 minutes, followed by addition of primary antibodies at indicated concentration in 5% BSA and incubated overnight at 4 deg on a rotator. Cells were washed 3× with PBS and incubated with secondary antibody in 5% BSA for 30 minutes at room temperature on a rotator. Cells were then washed 3× with PBS and incubated in DAPI for 5 minutes at a final concentration of 1 ug/mL in PBS. Cells were washed one more time with PBS and mounted on glass microscope slides using ProLong Glass Antifade Mountant.

Live cell imaging of tau aggregate formation, SRRM2 relocalization, SRRM2 C-terminus-halo, and 42-polyserine-halo. HEK293 tau biosensor cells were seeded and transfected with the appropriate vector in 24 well glass bottom plates with #1.5 cover glass at 2.5*10₅ cells/ml with or without 200 nM JF646-Halo ligand and allowed to grow overnight at 37° C. To counter stain the nuclei, Hoescht 33342 was added to the cell culture media and allowed to incubate for 15 minutes prior to imaging. For tau aggregate formation, cells were imaged on a Nikon Spinning Disc microscope every 30 minutes in the DAPI and GFP channels for 24 hours at 37° C. and 5% CO₂. For analysis of SRRM2 relocalization during tau aggregation, cells were imaged on an Opera Phenix High Content imaging system where images in the Cy5, GFP, and DAPI channels were acquired every 10 minutes for 48 hours at 37° C. and 5% CO₂. siRNA transfections. Cells were grown as described above and transfected with 5 pmol for 24 well and 25 pmol for 6 well using lipofectamine RNAiMAX and allowed to grow at 37° C. for 48 hours. Following 48 hours, tau seeds were transfected into the cells for 24 hours followed by fixation and imaging. NUP153 siRNAs were purchased from ThermoFisher (s19374) and SRRM2 siRNAs were purchased from ThermoFisher (s24003).

Fluorescence-activated cell sorting. HEK293T tau biosensor cells were transfected with plasmids 24 hours prior to seeding with tau brain homogenate and addition of TMRDirect Halo ligand to label exogenously expressed Halo-tagged fusion proteins. 24-hours post seeding cells were trypsinized, washed with PBS and filtered with 50 um nylon mesh filters prior to cell sorting. Sorting was performed with a BD FACSCelesta™ Cell Analyzer using the following filter sets: 561-585 (Halo), 405-450 (CFP) and 405-525 (FRET). Analysis was performed using FlowJo. Gating was performed in sequential steps, first sorting for cells, single cells, then gating based on Halo expression was performed by drawing a rectangular gate containing the highest expressing 10 percent of single cells. Following this gating step, a gate was drawn based on mock seeded cells to set a false FRET percentage at 1 as previously detailed (Furman et al., 2015). Integrated FRET Density was calculated as a product of the percentage of FRET positive cells and median fluorescence intensity. Statistical analysis was done with one-way anovas for each type of construct. Furman, J. L., Holmes, B. B., and Diamond, M. I. (2015). Sensitive detection of proteopathic seeding activity with FRET flow cytometry. J. Vis. Exp. 2015, 53205.

Image analysis using Ilastik and CellProfiler. To segment images into cytoplasmic tau aggregates, nuclear tau aggregates, nucleus, cytosol, and background, Ilastik was used with a minimum of five training images per condition per experiment. Images here hand annotated to show the location of the desired structures, this enabled the construction of a model that could then segment subsequent images. The original image and the image segmentation masks created by Ilastik were then used as inputs for a CellProfiler pipeline that calculated pixel intensity values of the 488 and 647 channels within the masked compartments. These compartmental measurements were used to calculate enrichment of SRRM2 in tau aggregates per image as follows: cytoplasmic tau aggregate enrichment=mean intensity within cytoplasmic tau aggregates per image/mean intensity within the cytosol per image. Nuclear tau aggregate enrichment=mean intensity within nuclear tau aggregates per image/mean intensity within the nuclei per image. To measure the integrated tau aggregate intensity, the total tau-YFP signal in cytoplasmic or nuclear tau aggregates was normalized by the number of nuclei identified in that image. This gave a measure of the size, intensity, and number of aggregates per cell within an image and can be used to compare tau aggregation across various conditions.

TABLE 3
Antibody information

| Antibody name | Manufacturer | Catalog # |
|---|---|---|
| Rabbit anti-SRRM2 | ThermoFisher | PA5-66827 |
| Rabbit anti-PNN (Pinin) | ThermoFisher | 18266-1-AP |
| Rabbit anti-SETD1A | Millipore Sigma | HPA058376 |

### Example 15. Supplemental Materials and Methods (Examples 8-13)

DNA Constructs. Fusion proteins and controls were cloned into pcDNA3.1 plasmids with a CMV promoter.

Cell culture and treatments. HEK293 biosensor cells stably expressing the 4R repeat domain of tau (K18) with the P301S mutation were purchased from ATCC (CRL-3275) (29). As previously described, cells were grown in 10% FBS, 0.2% penicillin-streptomycin at 37° C. with 5% carbon dioxide. For tau seeding, clarified brain homogenate was prepared as previously described. Tau brain homogenate was transfected into cells with Lipofectamine3000. For flow cytometry experiments tau brain homogenate was seeded at a final concentration of 0.5 ng/μl. For immunofluorescence experiments tau brain homogenate was seeded at a final concentration of 1.75 ng/μl. For re-infection experiments HEK tau biosensor cells were plated in 6-well format, transfected with plasmid (2.5 ug/well) and seeded with clarified tau brain homogenate with 24 hours between treatments and collection. Collected cell pellets were resuspended in PBS supplemented with protease inhibitor (Roche) and phosphatase inhibitor (Roche) and lysed with a 25G needles prior to a 1000xg spin for 3 minutes the remove cell debris. The supernatant was quantified with Bradford assay and used for seeding experiments at a final concentration of lug/24-well prior to flow cytometry.

Flow cytometry: Flow cytometry was performed as previously reported (16). Briefly, HEK293T tau biosensor cells were transfected with 500 ng of plasmid and seeded with clarified tau brain homogenate (0.5 ng/μl) at the timing as described. At the time of analysis, cells were trypsinized, washed in PBS and filtered through 40 um filters before analysis with BD FACS Celesta. TMRDirect Halo ligand (200 nM) was added 24 hours prior to analysis to label exogenously expressed Halo-tagged fusion proteins. 24 hours post-seeding, cells were trypsinized, washed with PBS, and filtered with 50 um nylon mesh filters prior to cell sorting. Sorting was performed with a BD FACSCelesta™ Cell Analyzer using the following filter sets: 561-585 (Halo), 405-450 (CFP), and 405-525 (FRET). Analysis was performed using FlowJo. Gating was performed in sequential steps, first sorting for cells, single cells, then (when applicable) gating based on Halo expression was performed. Lastly, gating for FRET+ cells was performed based on mock seeded cells to set a false FRET percentage at 1 as previously detailed (30). Integrated FRET Density was calculated as a product of the percentage of FRET-positive cells and median fluorescence intensity.

Western blot: For Western blotting, cell pellets were lysed in 2X SDS loading buffer, passed through a 25G syrine, and boiled. Protein extracts were run on 4-12 or 4-20% pre-case Tris-Glycine gels. Gels were then transferred to nitrocellulose membranes using the iBlot2 Transfer Device (Thermo Fisher). After transfer, membranes were blocked in 5% milk in Tris-buffered Saline with 0.1% Tween (TBS-T) for 1 hour, incubated with primary antibodies in TBS-T for 2 hours at roomtemperature, washed 3×10 minutes with TBST, incubated with secondary antibodies in TBS-T for 1 hour at room temperature, then washed 6×5 minutes with TBS-T before developing with Clarity Western ECL Substrate (Bio-rad).

Immunofluorescence: For immunofluorescence of Halo proteins, cells were treated 24 hours prior to collection with Janelia Fluor 646 Halo ligand (Promega). At the time of collection cells were fixed for 15 minutes in 4% PFA, then permeabilized with 0.5% Triton-X for 10 minutes. Cells were then blocked in 3% BSA/0.2% sodium azide for 1 hour prior to staining with DAPI. After 3×5 minute washes, cells were then mounted with ProLong Glass.

Confocal Microscopy and Image Quantification: Images were acquired on a Nikon spinning disk confocal microscope with a 100X objective. To quantify the fold enrichment of proteins in tau aggregates, a CellProfiler pipeline was generated to segment the cytoplasm and nuclei of individual cells. Next, cells that were positively transfected with Halo constructs were filtered based on intensity measurements. From this pool of Halo+ cells, cytoplasmic tau aggregates were then identified and segmented. Mean intensity measurements were taken and the fold enrichment reported for each cell as the mean intensity of Halo signal within tau aggregates/mean intensity of the remainder of the cytoplasm.

### REFERENCES

1. Lee V M, Goedert M, Trojanowski J Q. Neurodegenerative tauopathies. Annu Rev Neurosci. 2001;24:1121-59.
2. Mandelkow E M, Mandelkow E. Biochemistry and Cell Biology of Tau Protein in Neurofibrillary Degeneration. Cold Spring Harb Perspect Med. 2012 July;2(7):a006247.
3. Dujardin S, Hyman B T. Tau Prion-Like Propagation: State of the Art and Current Challenges. In: Takashima A, Wolozin B, Buee L, editors. Tau Biology [Internet]. Singapore: Springer; 2019 [cited 2023 Feb. 23]. p. 305-25. (Advances in Experimental Medicine and Biology). Available from: https://doi.org/10.1007/978-981-32-9358-8_23
4. Shorter J. Designer protein disaggregases to counter neurodegenerative disease. Curr Opin Genet Dev. 2017 June;44:1-8.
5. Jackrel M E, DeSantis M E, Martinez B A, Castellano L M, Stewart R M, Caldwell K A, et al. Potentiated Hsp104 variants antagonize diverse proteotoxic misfolding events. Cell. 2014 Jan. 16;156(0):170-82.
6. Guo L, Kim H J, Wang H, Monaghan J, Freyermuth F, Sung J C, et al. Nuclear-Import Receptors Reverse Aberrant Phase Transitions of RNA-Binding Proteins with Prionlike Domains. Cell. 2018 Apr. 19;173(3):677-692.e20.
7. Yoshizawa T, Ali R, Jiou J, Fung H Y J, Burke K A, Kim S J, et al. Nuclear Import Receptor Inhibits Phase Separation of FUS through Binding to Multiple Sites. Cell. 2018 Apr. 19;173(3):693-705.e22.
8. Qamar S, Wang G, Randle S J, Ruggeri F S, Varela J A, Lin J Q, et al. FUS Phase Separation Is Modulated by a Molecular Chaperone and Methylation of Arginine Cation-π Interactions. Cell. 2018 Apr. 19;173(3):720-734.e15.
9. Hofweber M, Hutten S, Bourgeois B, Spreitzer E, Niedner-Boblenz A, Schifferer M, et al. Phase Separation of FUS Is Suppressed by Its Nuclear Import Receptor and Arginine Methylation. Cell. 2018 Apr. 19;173(3):706-719.e13.
10. Johnson J O, Mandrioli J, Benatar M, Abramzon Y, Van Deerlin V M, Trojanowski J Q, et al. Exome sequencing reveals VCP mutations as a cause of familial ALS. Neuron. 2010 Dec. 9;68(5):857-64.
11. Watts G D J, Wymer J, Kovach M J, Mehta S G, Mumm S, Darvish D, et al. Inclusion body myopathy associated with Paget disease of bone and frontotemporal dementia is caused by mutant valosin-containing protein. Nat Genet. 2004 April;36(4):377-81.
12. Darwich N F, Phan J M, Kim B, Suh E, Papatriantafyllou J D, Changolkar L, et al. Autosomal dominant VCP hypomorph mutation impairs disaggregation of PHF-tau. Science. 2020 Nov. 20;370(6519):eaay8826.
13. Saha I, Yuste-Checa P, Da Silva Padilha M, Guo Q, Korner R, Holthusen H, et al. The AAA+ chaperone VCP disaggregates Tau fibrils and generates aggregate seeds in a cellular system. Nat Commun. 2023 Feb. 2;14(1):560.
14. Nachman E, Wentink A S, Madiona K, Bousset L, Katsinelos T, Allinson K, et al. Disassembly of Tau fibrils by the human Hsp70 disaggregation machinery generates small seeding-competent species. J Biol Chem. 2020 Jul. 10;295(28):9676-90.
15. Lester E, Ooi F K, Bakkar N, Ayers J, Woerman A L, Wheeler J, et al. Tau aggregates are RNA-protein assemblies that mislocalize multiple nuclear speckle components. Neuron. 2021 May 19;109(10):1675-1691.e9.
16. Lester E, Van Alstyne M, McCann K L, Reddy S, Cheng L Y, Kuo J, et al. Cytosolic condensates rich in polyserine define subcellular sites of tau aggregation. Proceedings of the National Academy of Sciences. 2023 Jan. 17;120(3):e2217759120.
17. Ryder B D, Wydorski P M, Hou Z, Joachimiak L A. Chaperoning shape-shifting tau in disease. Trends in Biochemical Sciences. 2022 Apr. 1;47(4):301-13.
18. Tanida I, Ueno T, Kominami E. LC3 and Autophagy. Methods Mol Biol. 2008;445:77-88.
19. Emerging functions of the VCP/p97 AAA-ATPase in the ubiquitin system |Nature Cell Biology [Internet]. [cited 2023 Feb. 26]. Available from: https://www.nature.com/articles/ncb2407
20. Zheng J, Cao Y, Yang J, Jiang H. UBXD8 mediates mitochondria-associated degradation to restrain apoptosis and mitophagy. EMBO reports. 2022 Oct. 6;23(10):e54859.
21. Lee J N, Kim H, Yao H, Chen Y, Weng K, Ye J. Identification of Ubxd8 protein as a sensor for unsaturated fatty acids and regulator of triglyceride synthesis. Proceedings of the National Academy of Sciences. 2010 Dec. 14;107(50):21424-9.
22. Ruggiano A, Foresti O, Carvalho P. ER-associated degradation: Protein quality control and beyond. Journal of Cell Biology. 2014 Mar. 17;204(6):869-79.
23. Schuberth C, Buchberger A. UBX domain proteins: major regulators of the AAA ATPase Cdc48/p97. Cell Mol Life Sci. 2008 Aug. 1;65(15):2360-71.
24. Dikic I, Wakatsuki S, Walters K J. Ubiquitin binding domains—from structures to functions. Nat Rev Mol Cell Biol. 2009 October; 10(10):659-71.
25. Zhang X, Xu Q, Zi Z, Liu Z, Wan C, Crisman L, et al. Programmable Extracellular Vesicles for Macromolecule Delivery and Genome Modifications. Dev Cell. 2020 Dec. 21;55(6):784-801.e9.
26. Gwon Y, Maxwell B A, Kolaitis R M, Zhang P, Kim H J, Taylor J P. Ubiquitination of G3BP1 mediates stress granule disassembly in a context-specific manner. Science. 2021 Jun. 25;372(6549):eabf6548.
27. Zhou H L, Geng C, Luo G, Lou H. The p97-UBXD8 complex destabilizes mRNA by promoting release of ubiquitinated HuR from mRNP. Genes Dev. 2013 May 1;27(9):1046-58.
28. McMillan P J, Strovas T J, Baum M, Mitchell B K, Eck R J, Hendricks N, et al. Pathological tau drives ectopic nuclear speckle scaffold protein SRRM2 accumulation in neuron cytoplasm in Alzheimer's disease. Acta Neuropathol Commun. 2021 Jun. 29;9(1):117.
29. Holmes B B, Furman J L, Mahan T E, Yamasaki T R, Mirbaha H, Eades W C, et al. Proteopathic tau seeding predicts tauopathy in vivo. Proceedings of the National Academy of Sciences. 2014 Oct. 14;111(41):E4376-85.
30. Furman J L, Holmes B B, Diamond M I. Sensitive Detection of Proteopathic Seeding Activity with FRET Flow Cytometry. J Vis Exp. 2015 Dec. 8;(106):e53205.

1. A composition for targeting a tau aggregates in a cell comprising a fusion peptide having:
a first domain comprising a tau targeting motif comprising an amino acid sequence encoding at least one polyserine repeat targeting a tau binding motif on a tau aggregate, or component thereof; and
a second domain encoding a heterologous peptide, or fragment thereof, having activity toward the tau aggregate, or component thereof.

2-3. (canceled)

4. The composition of claim 1, wherein said polyserine repeat comprises a polyserine repeat selected from:
one or more domains containing between 10-20 consecutive serine residues
one or more domains containing at least 20 consecutive serine residues;
one or more domains between 20 and 42 consecutive serine residues;
one or more domains between 20 and 50 consecutive serine residues; and
one or more domains containing at least 50 or more consecutive serine residues [.];
one or more domains containing a polyserine repeat between 20 and 42 consecutive serine residues;
one or more non-consecutive serine repeat domains;
one or more non-consecutive serine repeat domain comprising a peptide domain having at least 75% serine residues;
one or more non-consecutive serine repeat domain comprising a peptide domain having between 50-99% serine residues;
at least one polyserine repeat and at least one non-consecutive serine repeat; and
a polyserine repeat according to SEQ ID NO. 29.

5-8. (canceled)

9. The composition of claim 1, wherein said heterologous peptide is selected from: a peptide marker, a tag, E3 ubiquitin ligase, valosin-containing protein (VCP), RNase enzyme, transportin 3 (TNPO3), Fas associated factor family member 2 (FAF2), FAF2Min, RNaseL, nucleases, a ligand, a radioligand, fluorescent ligand, a kinases, a phosphatases, and acetyl transferases, a ubiquitin ligase, a ubiquitin segregase, a fluorophore, a VCP adaptor, or a combination of the same.

10-11. (canceled)

12. The composition of claim 1, wherein said heterologous peptide is selected from: SEQ ID NO. 38, SEQ ID NO. 39, SEQ ID NO. 44, or a fragment or variant thereof.

13-15. (canceled)

16. The composition of claim 1, wherein said tau aggregate comprises a pathogenic tau aggregate, or a monomeric tau peptide.

17. The composition of claim 1, wherein said polyserine repeat comprises a polyserine repeat selected from:
a peptide or fragment containing a polyserine repeat of Serine/Arginine Repetitive Matrix 2 (SRRM2); or
the C-terminal portion of SRRM2 containing a polyserine repeat; or
a peptide or fragment containing a polyserine repeat of PNN,
the C-terminal portion of a PNN containing a polyserine repeat; or
a peptide or fragment containing a one or more polyserine repeats of SETDIA, or a fragment or variant thereof.

18. (canceled)

19. The composition of claim 17, wherein the C-terminal portion of SRRM2 comprises a peptide encoding amino acids 2458-2752 of SEQ ID NO. 1.

20-21. (canceled)

22. The composition of claim 1, wherein the tau targeting motif comprising an amino acid sequence encoding at least one polyserine repeat targeting an intermediate molecule that binds a tau aggregate or a component thereof.

23-25. (canceled)

26. A composition for targeting a tau aggregates comprising a fusion peptide having:
a first domain comprising a tau targeting motif comprising an amino acid sequence encoding at least one polyserine repeat;
a second domain encoding a heterologous peptide that inhibits tau aggregates.

27-28. (canceled)

29. The composition of claim 26, wherein said polyserine repeat comprises a polyserine repeat selected from:
one or more domains containing between 10-20 consecutive serine residues
one or more domains containing at least 20 consecutive serine residues;
one or more domains between 20 and 42 consecutive serine residues;
one or more domains between 20 and 50 consecutive serine residues; and
one or more domains containing at least 50 or more consecutive serine residues;
one or more domains containing a polyserine repeat between 20 and 42 consecutive serine residues;
one or more non-consecutive serine repeat domains;
one or more non-consecutive serine repeat domain comprising a peptide domain having at least 75% serine residues;
one or more non-consecutive serine repeat domain comprising a peptide domain having between 50-99% serine residues;
at least one polyserine repeat and at least one non-consecutive serine repeat; and
a polyserine repeat according to SEQ ID NO. 29.

30-34. (canceled)

35. The composition of claim 26, wherein said polyserine repeat comprises a polyserine repeat selected from:
the C-terminal portion of SRRM2 according to the amino acid sequence SEQ ID NO. 40, or 46;
the C-terminal portion of PNN according to the amino acid sequence SEQ ID NO. 27;
a peptide or fragment containing a one or more polyserine repeats derived from SETDIA according to the amino acid sequence SEQ ID NO. 25, or
C-terminal portion of SRRM2 comprises a peptide encoding amino acids 2458-2752 of SEQ ID NO. 1.

36. (canceled)

37. The composition of claim 26, wherein the polyserine repeat comprises a polyserine repeat according to SEQ ID NO. 29.

38. The composition of claim 26, wherein said heterologous peptide is selected from: valosin-containing protein (VCP), transportin-3 (TNPO3), Fas associated factor family member 2 (FAF2), or a fragment or variant thereof.

39. The composition of claim 38, wherein the heterologous VCP comprises the amino acid sequence according to SEQ ID NO. 38, or a fragment or variant thereof.

40. (canceled)

41. The composition of claim 38, wherein the heterologous TNPO3 comprises the amino acid sequence according to SEQ ID NO. 39, or a fragment or variant thereof.

42. (canceled)

43. The composition of claim 38, wherein the heterologous FAF2 peptide comprises the amino acid sequence according to SEQ ID NO. 44, or a fragment or variant thereof.

44. (canceled)

45. The composition of claim 26, wherein said heterologous peptide comprises FAF2Min according to SEQ ID NO. 45, or a fragment or variant thereof.

46-59. (canceled)

60. The composition of claim 1, further comprising a pharmaceutically acceptable carrier.

61. The composition of claim 26, further comprising a pharmaceutically acceptable carrier.


SEQUENCE LISTING (47 sequences)

SEQ ID NO: 1 - protein, 2752 aa, Homo sapiens
MYNGIGLPTPRGSGTNGYVQRNLSLVRGRRGERPDYKGEEELRRLEAALVKRPNPDILDHERKRRVELRCLELEEMMEEQGYEEQQIQEKVATFRLMLLEKDVNPGGKEETPGQRPAVTETHQLAELNEKKNERLRAAFGISDSYVDGSSFDPQRRAREAKQPAPEPPKPYSLVRESSSSRSPTPKQKKKKKKKDRGRRSESSSPRRERKKSSKKKKHRSESESKKRKHRSPTPKSKRKSKDKKRKRSRSTTPAPKSRRAHRSTSADSASSSDTSRSRSRSAAAKTHTTALAGRSPSPASGRRGEGDAPFSEPGTTSTQRPSSPETATKQPSSPYEDKDKDKKEKSATRPSPSPERSSTGPEPPAPTPLLAERHGGSPQPLATTPLSQEPVNPPSEASPTRDRSPPKSPEKLPQSSSSESSPPSPQPTKVSRHASSSPESPKPAPAPGSHREISSSPTSKNRSHGRAKRDKSHSHTPSRRMGRSRSPATAKRGRSRSRTPTKRGHSRSRSPQWRRSRSAQRWGRSRSPQRRGRSRSPQRPGWSRSRNTQRRGRSRSARRGRSHSRSPATRGRSRSRTPARRGRSRSRTPARRRSRSRTPTRRRSRSRTPARRGRSRSRTPARRRSRTRSPVRRRSRSRSPARRSGRSRSRTPARRGRSRSRTPARRGRSRSRTPARRSGRSRSRTPARRGRSRSRTPRRGRSRSRSLVRRGRSHSRTPQRRGRSGSSSERKNKSRTSQRRSRSNSSPEMKKSRISSRRSRSLSSPRSKAKSRLSLRRSLSGSSPCPKQKSQTPPRRSRSGSSQPKAKSRTPPRRSRSSSSPPPKQKSKTPSRQSHSSSSPHPKVKSGTPPRQGSITSPQANEQSVTPQRRSCFESSPDPELKSRTPSRHSCSGSSPPRVKSSTPPRQSPSRSSSPQPKVKAIISPRQRSHSGSSSPSPSRVTSRTTPRRSRSVSPCSNVESRLLPRYSHSGSSSPDTKVKPETPPRQSHSGSISPYPKVKAQTPPGPSLSGSKSPCPQEKSKDSLVQSCPGSLSLCAGVKSSTPPGESYFGVSSLQLKGQSQTSPDHRSDTSSPEVRQSHSESPSLQSKSQTSPKGGRSRSSSPVTELASRSPIRQDRGEFSASPMLKSGMSPEQSRFQSDSSSYPTVDSNSLLGQSRLETAESKEKMALPPQEDATASPPRQKDKFSPFPVQDRPESSLVFKDTLRTPPRERSGAGSSPETKEQNSALPTSSQDEELMEVVEKSEEPAGQILSHLSSELKEMSTSNFESSPEVEERPAVSLTLDQSQSQASLEAVEVPSMASSWGGPHFSPEHKELSNSPLRENSFGSPLEFRNSGPLGTEMNTGFSSEVKEDLNGPFLNQLETDPSLDMKEQSTRSSGHSSSELSPDAVEKAGMSSNQSISSPVLDAVPRTPSRERSSSASSPEMKDGLPRTPSRRSRSGSSPGLRDGSGTPSRHSLSGSSPGMKDIPRTPSRGRSECDSSPEPKALPQTPRPRSRSPSSPELNNKCLTPQRERSGSESSVDQKTVARTPLGQRSRSGSSQELDVKPSASPQERSESDSSPDSKAKTRTPLRQRSRSGSSPEVDSKSRLSPRRSRSGSSPEVKDKPRAAPRAQSGSDSSPEPKAPAPRALPRRSRSGSSSKGRGPSPEGSSSTESSPEHPPKSRTARRGSRSSPEPKTKSRTPPRRRSSRSSPELTRKARLSRRSRSASSSPETRSRTPPRHRRSPSVSSPEPAEKSRSSRRRRSASSPRTKTTSRRGRSPSPKPRGLQRSRSRSRREKTRTTRRRDRSGSSQSTSRRRQRSRSRSRVTRRRRGGSGYHSRSPARQESSRTSSRRRRGRSRTPPTSRKRSRSRTSPAPWKRSRSRASPATHRRSRSRTPLISRRRSRSRTSPVSRRRSRSRTSVTRRRSRSRASPVSRRRSRSRTPPVTRRRSRSRTPTTRRRSRSRTPPVTRRRSRSRTPPVTRRRSRSRTSPITRRRSRSRTSPVTRRRSRSRTSPVTRRRSRSRTSPVTRRRSRSRTPPAIRRRSRSRTPLLPRKRSRSRSPLAIRRRSRSRTPRTARGKRSLTRSPPAIRRRSASGSSSDRSRSATPPATRNHSGSRTPPVALNSSRMSCFSRPSMSPTPLDRCRSPGMLEPLGSSRTPMSVLQQAGGSMMDGPGPRIPDHQRTSVPENHAQSRIALALTAISLGTARPPPSMSAAGLAARMSQVPAPVPLMSLRTAPAANLASRIPAASAAAMNLASARTPAIPTAVNLADSRTPAAAAAMNLASPRTAVAPSAVNLADPRTPTAPAVNLAGARTPAALAALSLTGSGTPPTAANYPSSSRTPQAPASANLVGPRSAHATAPVNIAGSRTAAALAPASLTSARMAPALSGANLTSPRVPLSAYERVSGRTSPPLLDRARSRTPPSAPSQSRMTSERAPSPSSRMGQAPSQSLLPPAQDQPRSPVPSAFSDQSRCLIAQTTPVAGSQSLSSGAVATTTSSAGDHNGMLSVPAPGVPHSDVGEPPASTGAQQPSALAALQPAKERRSSSSSSSSSSSSSSSSSSSSSSSSSGSSSSDSEGSSLPVQPEVALKRVPSPTPAPKEAVREGRPPEPTPAKRKRRSSSSSSSSSSSSSSSSSSSSSSSSSSSSSSSSSSSSSSSSSSPSPAKPGPQALPKPASPKKPPPGERRSRSPRKPIDSLRDSRSLSYSPVERRRPSPQPSPRDQQSSSSERGSRRGQRGDSRSPSHKRRRETPSPRPMRHRSSRSP

SEQ ID NO: 2 - DNA, 9044 bp, Homo sapiens
  source 1..9044: mol_type = genomic DNA
gtgctgacgtgcagcgcggcccaggcggggtgcgagtggcgcagttggagcccgttgcggcccctgaggaagcgaggaggcgtcggcgtcggctgaggcgggcggaccggcgaggcgaggcggcggccccaggcccgagggactcgggagctcgagcagcggcggcggcaagacctctccccctcggaggcggcgggcggaggcggcgggagcggtggtgccccccccgggcacggggccatgtacaacgggatcgggctgccgacgccccggggcagcggcaccaacggctacgtccagcgcaacctgtccctggtgcggggccgccggggtgagcggcctgactacaagggagaggaggaactgcggcgcctggaggctgccctggtgaagcggcctaatcctgacatcctggaccacgagcgcaagcggcgcgtcgagctgcgatgcctcgagctggaggagatgatggaagagcaggggtacgaggaacagcaaattcaggaaaaagtggcgacctttcgactcatgttgctggagaaggatgtgaaccctgggggcaaggaggagaccccagggcagaggccagcggtcacggagactcaccagttggcagaattaaatgagaagaagaatgaaagactccgtgctgcctttggcatcagtgattcttacgtagatggcagctcttttgatcctcagcgtcgtgcccgagaagctaaacaaccagctcctgagcctcccaaaccttacagccttgttcgggagtctagcagttctcgctcaccaaccccaaagcagaagaagaagaaaaagaagaaagatagaggacgcaggtcagagagcagctctcctcgacgggagagaaagaaaagctcaaagaagaagaagcacaggtcagaatctgagtccaagaaacgtaagcataggtctcccactccaaagagcaaacgtaaatctaaggacaaaaagcgaaagcggtctcgaagtacaacaccagcccccaagagccgccgggcccaccgttcaacttctgctgactctgcttcctcctccgatacttcccgcagtcggtctcgaagtgctgcagctaaaactcatacaactgccttggctgggcgaagtccttcccctgcttcagggcgacgcggggagggagatgcgcctttcagtgaaccaggtactaccagcacacaacggcctagtagcccggagactgctacgaaacagcctagcagcccttatgaagacaaagataaagacaagaaggagaaatctgcaactcgacctagcccctctccggaaaggagcagcacaggcccagaaccacctgctcccactccgctccttgctgagcgacatggcggctccccacaaccccttgcaaccacccccttaagccaggagccagtgaaccccccatctgaggcctctccaactcgggaccgttcaccacctaagtctcccgagaaacttccccagtcttcttcctcagagagcagcccaccatcccctcaacctaccaaagtttctcggcatgccagctcttccccagaaagtcctaaacctgctccagctccagggtcccaccgagagatttcttcttctcccacatctaagaatcgctcacatggccgagcaaaacgggataaatcacattctcataccccctcccgtaggatggggaggtcccgtagccctgccaccgctaagagagggcgatctcggtctcgaacccctaccaagagaggtcattctcgatcccgatctccccagtggcgtaggtccaggtctgcacagaggtggggaagatctagaagcccccagcgacgtggccgctctaggtctcctcagcgaccaggctggtctaggagcagaaatacccagagaagaggcaggtctaggtcagcaaggcgagggaggtcccactctagatccccagccactaggggtagatctcgttctagaacaccagcccgccggggcaggtcccgctctagaacacctgccaggcggagatcacgatccagaactcccaccaggcgtaggtctcggtctagaacaccagcccggaggggcaggtctcggtctagaacacctgctaggcgcagatctaggacccgatcaccagtacgacgcaggtctcgtagtagatcaccagccaggagaagtggcaggtcacgctctagaaccccagctagacgtggccgctcacgctccagaaccccagccagacgtggccgctcacgctctagaaccccagctagacgcagtggtcgctcacgctccagaacaccagccaggagagggaggtctcggtctaggacaccaagacgaggaagatcccgcagtagaagcttagttagacgtggaagatctcactctagaacacctcaaagaagaggcagatctggctcatcttcagagcggaaaaacaaatccagaacatctcaaagaagaagcaggtccaattcaagcccagaaatgaagaaatctcgcatttcttcaaggcggagcaggtctctctcttcaccacggtccaaagcaaaatctcgcttgtctttgaggcgcagcctttcagggtcttccccatgccctaaacaaaagtcacagacaccacccaggcgcagtcgctctggatcctcccaacctaaagctaaatctagaacgccacccagacgcagtcgctccagttcttctccgccacctaaacagaaatctaagacaccatcaagacaaagtcattccagttcatctcctcatcctaaagtgaaatctggaacaccaccgaggcaagggtccataacaagtccccaggccaatgagcaatctgtaacgccacagagacggagctgttttgaatcatcacctgaccctgagttgaaatctaggaccccttctagacatagctgctcagggtcctctcctcctagagtgaaatctagcacacctcccagacagagcccatctaggtcatcatctccacaacccaaagtgaaggcaataatatcaccaagacaaagaagccattctggctcctcttctccaagtcctagtagggtgacgtcgagaacaactccacggcgaagcagatcagtatctccctgctccaatgtggaatccagattgttgccaagatacagtcattctgggtcctcctcaccagataccaaagtgaaacctgaaacaccgccaagacaaagtcactcagggtctatttcaccataccccaaagtaaaggcccaaactccaccggggccaagtctttctggatcaaagtcaccatgtccccaagagaagtctaaagactcactagttcaaagttgccctggatccctctctctctgtgcaggagtaaaatctagcacaccaccaggcgagagctattttggtgtctcatctctgcaactgaaaggacaatctcaaacttcaccagaccacagatctgatacttcaagtccagaagtgagacagagtcattcagaatcaccatctctgcagagcaaatctcaaacatcacctaagggaggtcggtccaggtcttcatctccagtcactgagctggcatccagatctccaataagacaagatagaggtgagttctcagcgagtcctatgttgaaatctggaatgtctcctgagcagagcaggttccagtctgactcttcttcatatcctacagtggactcgaattctctcttggggcagagtagattggagactgctgaatcaaaagagaaaatggccttaccccctcaggaggatgctactgcatcacctcctagacagaaagacaaatttagtccctttccagtacaggataggcctgagtcttcactggtattcaaagacacacttagaaccccgccaagggaaagaagtggtgctgggtcatctccagaaacaaaagagcaaaatagtgcattgcctacgtcaagccaagatgaagagttaatggaggtggtagagaagtctgaagaacccgcaggccaaatcctgtctcatttgtcttcagaacttaaagaaatgtccacaagtaactttgaatcatctcctgaagtagaagaaaggcctgctgtgtctttgactcttgatcagagccagtcacaggcttctttggaagcagtagaagtcccttcaatggcctcatcttggggtgggccacatttttctccagaacataaagaactgtctaactccccactcagggagaacagctttggatcacctttagaatttagaaactcaggcccacttggtacagaaatgaatactggattttcttctgaggttaaagaagatttgaatggaccgtttcttaatcagctggaaacagatccatctctagacatgaaagaacaatcgacaagatcctctggacacagcagttctgagttatccccagatgcagtggaaaaggcagggatgtcttcaaatcagagcatctcttcacctgtgcttgatgctgtacccagaacaccctcgagagaaagaagtagttctgcatcttctcctgaaatgaaagatggtttacccagaactccatcaaggagaagcaggtctgggtcttctccaggacttagagatgggtctgggactccctcgaggcacagcctgtctgggtcctctcctggaatgaaagatatacctagaacgccatctagagggagaagcgaatgtgattcttccccagaaccgaaagctttgcctcagactcctaggccgaggagtcgttctccatcatccccagagctcaacaacaagtgtcttaccccccagagagaaagaagcgggtcagaatcatcagttgatcagaaaactgtggctcggactcccctggggcagagaagtcgttcgggatcctctcaagaacttgatgtgaaacccagtgcatcccctcaggaaagaagtgagtcagactcttctccagattctaaagccaagacaagaaccccacttcggcagaggagtcggtctggatcatctccagaggttgacagcaaatctcgactatcccctcggcgcagtaggtctggttcctcccctgaagtgaaagataagccaagagcagcacccagggcacagagtggttctgattcctctcctgaacctaaagctccagcccctcgggcccttcccagacgaagcagatcaggttcatcaagcaaaggcagaggcccttctcctgaaggaagcagcagtaccgagtcctctcctgaacatccgcccaaatccagaactgctcgcagaggttccaggtcatcaccagagcccaagaccaagtctcgtacaccacctcgacgtcgcagctctcgatcatctccggagctaacaaggaaggccagactgtcccgtagaagccgctctgcctcatcctcaccagaaactcgctctagaactcccccaaggcaccggagaagtccctcagtgtcttccccggagccagccgaaaaatcgaggtcttcacgccgacggcgctcagcttcatctccacgcactaagacaacctcaaggagaggccgctctccttcgccaaagcctcgtggactccagaggtcccgttcccgctcaaggagagagaaaacaagaacaacccgacgtcgagataggtctggatcttctcagtcaacctctcggcgaagacagcggagccggtcaaggtcgcgggttactcggcggcggaggggaggctctggttatcactcaaggtcacctgcccggcaggaaagttcccggacctcctctcgacgccgaagaggccgctctcggacacccccaaccagtcggaagcgttctcgctcacgcacatcaccagccccgtggaaacgctctagatctcgagcctctccagccactcaccggcgatccaggtccagaacccccctgataagccgacgtaggtccagatctcgaacttcaccagtcagccggagacggtcaaggtccaggacttcagtgactcgacgaagatcccggtcaagagcatccccagtgagcagaaggcgatccagatccagaacgccaccagtaacccgccgtcgttcaaggtctagaacgccaacaacacgccgccgctcccgttctagaactccaccagtgactcgcagaaggtccagatccaggactccaccagtaaccaggaggcgatctcgaagcagaacttcgcctatcactcgcagaagatcaagatccagaacatctccggtcacccgaaggagatctcgatctcgcacatctccagtaactcgaagaaggtcccgctctcgaacctcaccagtgacacgccgccgctctaggtcccggacacctccagctattcggcgccgctctagatctcgaacgccactgttaccacgcaaacgttctcgaagtcgctcaccacttgctatccgccgccgctccagatcccgtactccacgaacagctcggggtaaacggtccttaacaagatctcctccagccatccgcaggcgttctgcatctggaagtagttctgatcgttcacgatctgctactcctccagcaacaagaaatcattctggttcacggacacctccagtagcactcaacagttccagaatgagctgcttcagtcgtcctagcatgtccccaacacctcttgatcgctgcagatcacctggaatgcttgaaccccttggcagctctagaacacccatgtctgtcctgcagcaagccggcggctccatgatggatggtccaggtccccgaatacctgaccaccagagaacatctgtgccagaaaatcatgctcagtccaggattgcacttgccctgacagctatcagtcttggcaccgctcggcctcctccgtccatgtctgctgctggccttgctgcaagaatgtcccaggttccagccccggtgcctctcatgagtctcagaaccgcaccagcagccaaccttgccagcaggattcctgcagcctctgcggcagccatgaacctagccagcgccaggacacctgccattccaacagcagtgaacctggctgactctcgaacgccagctgcagcagcggccatgaacttggccagccccagaacagcggtggcaccttcggctgtgaacctggctgaccctcgcactcccacagccccagctgtgaacctagcaggggccagaaccccagctgccttggcagctctgagtctcacaggctctggcacaccaccaactgctgcaaactatccctccagctccagaacaccacaggctccagcctctgcaaacctggtgggtcctcggtctgcacatgccacagctcctgtgaatattgccggctccagaaccgccgcagccttggcccccgcgagcctcaccagtgctaggatggctccagcattgtctggtgcaaacctcaccagccccagggtgcccctttctgcctacgagcgtgtcagtggcagaacctcaccaccgctccttgaccgagctaggtccagaacaccaccgtctgccccaagccaatctaggatgacctctgaacgggctccctccccttcctctagaatgggccaggctccttcacagtctcttctccctccagcacaggatcagccgaggtctcctgtgccttctgctttttcagaccaatcccgttgtttgattgcccagaccacccctgtagcagggtctcagtccctttcctctggggcagtggcaacgaccacgtcctctgctggtgatcacaatggcatgctctctgtccctgcccctggggtgccccactctgatgtgggggagccacctgcctctactggggcccagcagccttctgcattagccgccctgcagccagcaaaggagcggcggagttcctcctcgtcgtcgtcgtcctctagctcctcctcttcttcatcatcgtcgtcgtcgtcctcctcctcctctggctccagttctagtgactcagagggctctagccttcctgtgcaacctgaggtggcactgaagagggtccccagccccaccccagccccaaaggaggctgttcgagagggacgtcctccggagccaaccccagccaaacggaagaggcgctctagcagttccagttccagctcctcctcttcatcttcctcctcctcctcctcctcctcttcttcctcctcctcttcctcttcttcttcttcctcctcatcttcctcctcctcgtcgtcttcctccccttcccctgctaagcctggccctcaggccttgcccaaacctgcaagccccaagaagccaccccctggcgagcggaggtcccgcagcccccggaagccaatagactccctcagggactctcggtccctcagctactcgcctgtggagcgtcgccgtccctcgccccagccctcaccacgggaccagcagagcagcagcagtgagcggggttcccggagaggccagcgtggggacagccgctcccccagccacaagcgcaggagggagacacctagccctcggcccatgagacaccgctcctccaggtctccataaattgtctttgggggattccaccacacccaatgctctggagccacaaggagtgtcccttcttccccagcagagccgtgggagggtccttgtctgctctcctttgaaccttggcagcccttggatggagggctccctttccctccccttttttttttctttgttcctgtgaaatgttaatctccgtgagttcttcctggttcatgtgttctggggggtttggggtgggagggaatgcagatgggagttgggggaggggaggatacagttcaggataccccagcctggagtcagggccagggaggcatggccccacttgtatccagaagttcccaggggtgattgtgatggtggttgggactggaggttgtataaggtgttcttggaaggaaggggcaggagttggaattagttggtccctactgtcccccatgaggttgtgaacccctccccccaacttttcatgtttcttaaaggcattttggttttttaaaatctgtacagcaagagcaactttttctgtcaaataaaaatgagaaatgcaggaa

SEQ ID NO: 3 - DNA, 25 bp, synthetic construct
  source 1..25: mol_type = other DNA
caccgccatgagacaccgctcctcc

SEQ ID NO: 4 - DNA, 25 bp, synthetic construct
  source 1..25: mol_type = other DNA
aaacggaggagcggtgtctcatggc

SEQ ID NO: 5 - DNA, 24 bp, synthetic construct
  source 1..24: mol_type = other DNA
caccgctagagcgcctcttccgtt

SEQ ID NO: 6 - DNA, 24 bp, synthetic construct
  source 1..24: mol_type = other DNA
aaacaacggaagaggcgctctagc

SEQ ID NO: 7 - DNA, 24 bp, synthetic construct
  source 1..24: mol_type = other DNA
caccgattggtctgaaaaagcaga

SEQ ID NO: 8 - DNA, 24 bp, synthetic construct
  source 1..24: mol_type = other DNA
aaactctgctttttcagaccaatc

SEQ ID NO: 9 - DNA, 24 bp, synthetic construct
  source 1..24: mol_type = other DNA
aaactctgctttttcagaccaatc

SEQ ID NO: 10 - DNA, 24 bp, synthetic construct
  source 1..24: mol_type = other DNA
aaacatgtctgtcctgcagcaagc

SEQ ID NO: 11 - DNA, 24 bp, synthetic construct
  source 1..24: mol_type = other DNA
caccgtttccagctgattaagaaa

SEQ ID NO: 12 - DNA, 24 bp, synthetic construct
  source 1..24: mol_type = other DNA
aaactttcttaatcagctggaaac

SEQ ID NO: 13 - DNA, 24 bp, synthetic construct
  source 1..24: mol_type = other DNA
caccggcgttctagatttagcttt

SEQ ID NO: 14 - DNA, 24 bp, synthetic construct
  source 1..24: mol_type = other DNA
aaacaaagctaaatctagaacgcc

SEQ ID NO: 15 - DNA, 24 bp, synthetic construct
  source 1..24: mol_type = other DNA
caccggtctaggtcagcaaggcga

SEQ ID NO: 16 - DNA, 24 bp, synthetic construct
  source 1..24: mol_type = other DNA
aaactcgccttgctgacctagacc

SEQ ID NO: 17 - DNA, 24 bp, synthetic construct
  source 1..24: mol_type = other DNA
caccgctggcatgccgagaaactt

SEQ ID NO: 18 - DNA, 24 bp, synthetic construct
  source 1..24: mol_type = other DNA
aaacaagtttctcggcatgccagc

SEQ ID NO: 19 - DNA, 46 bp, synthetic construct
  source 1..46: mol_type = other DNA
ttaaacgggccctctagtctagatcaaccggaaatctccagagtag

SEQ ID NO: 20 - DNA, 49 bp, synthetic construct
  source 1..49: mol_type = other DNA
ggtggaattctgcagatatcgccaccatggaaatcggtactggctttcc

SEQ ID NO: 21 - DNA, 49 bp, synthetic construct
  source 1..49: mol_type = other DNA
ggtggaattctgcagatatcgccaccatgaagcctggccctcaggcctt

SEQ ID NO: 22 - DNA, 46 bp, synthetic construct
  source 1..46: mol_type = other DNA
ggtggaattctgcagatatcgccaccatgcgctctagcagttccag

SEQ ID NO: 23 - DNA, 49 bp, synthetic construct
  source 1..49: mol_type = other DNA
ggtggaattctgcagatatcgccaccatgcaatcccgttgtttgattgc

SEQ ID NO: 24 - protein, 717 aa, Homo sapiens
MAVAVRTLQEQLEKAKESLKNVDENIRKLTGRDPNDVRPIQARLLALSGPGGGRGRGSLLLRRGFSDSGGGPPAKQRDLEGAVSRLGGERRTRRESRQESDPEDDDVKKPALQSSVVATSKERTRRDLIQDQNMDEKGKQRNRRIFGLLMGTLQKFKQESTVATERQKRRQEIEQKLEVQAEEERKQVENERRELFEERRAKQTELRLLEQKVELAQLQEEWNEHNAKIIKYIRTKTKPHLFYIPGRMCPATQKLIEESQRKMNALFEGRRIEFAEQINKMEARPRRQSMKEKEHQVVRNEEQKAEQEEGKVAQREEELEETGNQHNDVEIEEAGEEEEKEIAIVHSDAEKEQEEEEQKQEMEVKMEEETEVRESEKQQDSQPEEVMDVLEMVENVKHVIADQEVMETNRVESVEPSENEASKELEPEMEFEIEPDKECKTLSPGKENVSALDMEKESEEKEEKESEPQPEPVAQPQPQSQPQLQLQSQSQPVLQSQPPSQPEDLSLAVLQPTPQVTQEQGHLLPERKDFPVESVKLTEVPVEPVLTVHPESKSKTKTRSRSRGRARNKTSKSRSRSSSSSSSSSSSTSSSSGSSSSSGSSSSRSSSSSSSSTSGSSSRDSSSSTSSSSESRSRSRGRGHNRDRKHRRSVDRKRRDTSGLERSHKSSKGGSSRDTKGSKDKNSRSDRKRSISESSRSGKRSSRSERDRKSDRKDKRR

SEQ ID NO: 25 - protein, 1707 aa, Homo sapiens
MDQEGGGDGQKAPSFQWRNYKLIVDPALDPALRRPSQKVYRYDGVHFSVNDSKYIPVEDLQDPRCHVRSKNRDFSLPVPKFKLDEFYIGQIPLKEVTFARLNDNVRETFLKDMCRKYGEVEEVEILLHPRTRKHLGLARVLFTSTRGAKETVKNLHLTSVMGNIIHAQLDIKGQQRMKYYELIVNGSYTPQTVPTGGKALSEKFQGSGAATETAESRRRSSSDTAAYPAGTTAVGTPGNGTPCSQDTSFSSSRQDTPSSFGQFTPQSSQGTPYTSRGSTPYSQDSAYSSSTTSTSFKPRRSENSYQDAFSRRHFSASSASTTASTAIAATTAATASSSASSSSLSSSSSSSSSSSSSQFRSSDANYPAYYESWNRYQRHTSYPPRRATREEPPGAPFAENTAERFPPSYTSYLPPEPSRPTDQDYRPPASEAPPPEPPEPGGGGGGGGPSPEREEVRTSPRPASPARSGSPAPETTNESVPFAQHSSLDSRIEMLLKEQRSKFSFLASDTEEEEENSSMVLGARDTGSEVPSGSGHGPCTPPPAPANFEDVAPTGSGEPGATRESPKANGQNQASPCSSGDDMEISDDDRGGSPPPAPTPPQQPPPPPPPPPPPPPYLASLPLGYPPHQPAYLLPPRPDGPPPPEYPPPPPPPPHIYDFVNSLELMDRLGAQWGGMPMSFQMQTQMLTRLHQLRQGKGLIAASAGPPGGAFGEAFLPFPPPQEAAYGLPYALYAQGQEGRGAYSREAYHLPMPMAAEPLPSSSVSGEEARLPPREEAELAEGKTLPTAGTVGRVLAMLVQEMKSIMQRDLNRKMVENVAFGAFDQWWESKEEKAKPFQNAAKQQAKEEDKEKTKLKEPGLLSLVDWAKSGGTTGIEAFAFGSGLRGALRLPSFKVKRKEPSEISEASEEKRPRPSTPAEEDEDDPEQEKEAGEPGRPGTKPPKRDEERGKTQGKHRKSFALDSEGEEASQESSSEKDEEDDEEDEEDEDREEAVDTTKKETEVSDGEDEESDSSSKCSLYADSDGENDSTSDSESSSSSSSSSSSSSSSSSSSSSSSSESSSEDEEEEERPAALPSASPPPREVPVPTPAPVEVPVPERVAGSPVTPLPEQEASPARPAGPTEESPPSAPLRPPEPPAGPPAPAPRPDERPSSPIPLLPPPKKRRKTVSFSAIEVVPAPEPPPATPPQAKFPGPASRKAPRGVERTIRNLPLDHASLVKSWPEEVSRGGRSRAGGRGRLTEEEEAEPGTEVDLAVLADLALTPARRGLPALPAVEDSEATETSDEAERPRPLLSHILLEHNYALAVKPTPPAPALRPPEPVPAPAALFSSPADEVLEAPEVVVAEAEEPKPQQLQQQREEGEEEGEEEGEEEEEESSDSSSSSDGEGALRRRSLRSHARRRRPPPPPPPPPPRAYEPRSEFEQMTILYDIWNSGLDSEDMSYLRLTYERLLQQTSGADWLNDTHWVHHTITNLTTPKRKRRPQDGPREHQTGSARSEGYYPISKKEKDKYLDVCPVSARQLEGVDTQGTNRVLSERRSEQRRLLSAIGTSAIMDSDLLKLNQLKFRKKKLRFGRSRIHEWGLFAMEPIAADEMVIEYVGQNIRQMVADMREKRYVQEGIGSSYLFRVDHDTIIDATKCGNLARFINHCCTPNCYAKVITIESQKKIVIYSKQPIGVDEEITYDYKFPLEDNKIPCLCGTESCRGSLN

SEQ ID NO: 26 - DNA, 201 bp, synthetic construct
  source 1..201: mol_type = other DNA
atgtcaaaatccagatctcgaagctcatccagcagcagttcaagtagcagttctacctctagttccagtgggtcaagctcctccagtggatcatcctccagtcggagctcatccagctctagctccagcacgagtgggtcaagctctcgggactcatcatcatctacttcaagctctagtgagtcaagaagccggagtcgc

SEQ ID NO: 27 - protein, 67 aa, Homo sapiens
MSKSRSRSSSSSSSSSSSTSSSSGSSSSSGSSSSRSSSSSSSSTSGSSSRDSSSSTSSSSESRSRSR

SEQ ID NO: 28 - DNA, 129 bp, synthetic construct
  source 1..129: mol_type = other DNA
atgtctagttcttcaagctccagctcaagtagttccagttcctctagtagctcctcatccagctcttcttcatcatccagcagctctagcagtagttcttccagcagctctagctcctcctcatcttcc

SEQ ID NO: 29 - protein, 42 aa, synthetic construct
SSSSSSSSSSSSSSSSSSSSSSSSSSSSSSSSSSSSSSSSSS

SEQ ID NO: 30 - DNA, 3468 bp, synthetic construct
  source 1..3468: mol_type = other DNA
atggcttctggagccgattcaaaaggtgatgacctatcaacagccattctcaaacagaagaaccgtcccaatcggttaattgttgatgaagccatcaatgaggacaacagtgtggtgtccttgtcccagcccaagatggatgaattgcagttgttccgaggtgacacagtgttgctgaaaggaaagaagagacgagaagctgtttgcatcgtcctttctgatgatacttgttctgatgagaagattcggatgaatagagttgttcggaataaccttcgtgtacgcctaggggatgtcatcagcatccagccatgccctgatgtgaagtacggcaaacgtatccatgtgctgcccattgatgacacagtggaaggcattactggtaatctcttcgaggtataccttaagccgtacttcctggaagcgtatcgacccatccggaaaggagacatttttcttgtccgtggtgggatgcgtgctgtggagttcaaagtggtggaaacagatcctagcccttattgcattgttgctccagacacagtgatccactgcgaaggggagcctatcaaacgagaggatgaggaagagtccttgaatgaagtagggtatgatgacattggtggctgcaggaagcagctagctcagataaaggagatggtggaactgcccctgagacatcctgccctctttaaggcaattggtgtgaagcctcctagaggaatcctgctttacggacctcctggaacaggaaagaccctgattgctcgagctgtagcaaatgagactggagccttcttcttcttgatcaatggtcctgagatcatgagcaaattggctggtgagtctgagagcaaccttcgtaaagcctttgaggaggctgagaagaatgctcctgccatcatcttcattgatgagctagatgccatcgctcccaaaagagagaaaactcatggcgaggtggagcggcgcattgtatcacagttgttgaccctcatggatggcctaaagcagagggcacatgtgattgttatggcagcaaccaacagacccaacagcattgacccagctctacggcgatttggtcgctttgacagggaggtagatattggaattcctgatgctacaggacgcttagagattcttcagatccataccaagaacatgaagctggcagatgatgtggacctggaacaggtagccaatgagactcacgggcatgtgggtgctgacttagcagccctgtgctcagaggctgctctgcaagccatccgcaagaagatggatctcattgacctagaggatgagaccattgatgccgaggtcatgaactctctagcagttactatggatgacttccggtgggccttgagccagagtaacccatcagcactgcgggaaaccgtggtagaggtgccacaggtaacctgggaagacatcgggggcctagaggatgtcaaacgtgagctacaggagctggtccagtatcctgtggagcacccagacaaattcctgaagtttggcatgacaccttccaagggagttctgttctatggacctcctggctgtgggaaaactttgttggccaaagccattgctaatgaatgccaggccaacttcatctccatcaagggtcctgagctgctcaccatgtggtttggggagtctgaggccaatgtcagagaaatctttgacaaggcccgccaagctgccccctgtgtgctattctttgatgagctggattcgattgccaaggctcgtggaggtaacattggagatggtggtggggctgctgaccgagtcatcaaccagatcctgacagaaatggatggcatgtccactaaaaaaaatgtgttcatcattggcgctaccaaccggcctgacatcattgatcctgccatcctcagacctggccgtcttgatcagctcatctacatcccacttcctgatgagaagtcccgtgttgccatcctcaaggctaacctgcgcaagtccccagttgccaaggatgtggacttggagttcctggctaaaatgactaatggcttctctggagctgacctgacagagatttgccagcgtgcttgcaagctggccatccgtgaatccatcgagagtgagattaggcgagaacgagagaggcagacaaacccatcagccatggaggtagaagaggatgatccagtgcctgagatccgtcgagatcactttgaagaagccatgcgctttgcgcgccgttctgtcagtgacaatgacattcggaagtatgagatgtttgcccagacccttcagcagagtcggggctttggcagcttcagattcccttcagggaaccagggtggagctggccccagtcagggcagtggaggcggcacaggtggcagtgtatacacagaagacaatgatgatgacctgtatggcgatatctctagttcttcaagctccagctcaagtagttccagttcctctagtagctcctcatccagctcttcttcatcatccagcagctctagcagtagttcttccagcagctctagctcctcctcatcttccggggggtctggtggcagtggagggggatccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggttga

SEQ ID NO: 31 - protein, 1155 aa, synthetic construct
MASGADSKGDDLSTAILKQKNRPNRLIVDEAINEDNSVVSLSQPKMDELQLFRGDTVLLKGKKRREAVCIVLSDDTCSDEKIRMNRVVRNNLRVRLGDVISIQPCPDVKYGKRIHVLPIDDTVEGITGNLFEVYLKPYFLEAYRPIRKGDIFLVRGGMRAVEFKVVETDPSPYCIVAPDTVIHCEGEPIKREDEEESLNEVGYDDIGGCRKQLAQIKEMVELPLRHPALFKAIGVKPPRGILLYGPPGTGKTLIARAVANETGAFFFLINGPEIMSKLAGESESNLRKAFEEAEKNAPAIIFIDELDAIAPKREKTHGEVERRIVSQLLTLMDGLKQRAHVIVMAATNRPNSIDPALRRFGRFDREVDIGIPDATGRLEILQIHTKNMKLADDVDLEQVANETHGHVGADLAALCSEAALQAIRKKMDLIDLEDETIDAEVMNSLAVTMDDFRWALSQSNPSALRETVVEVPQVTWEDIGGLEDVKRELQELVQYPVEHPDKFLKFGMTPSKGVLFYGPPGCGKTLLAKAIANECQANFISIKGPELLTMWFGESEANVREIFDKARQAAPCVLFFDELDSIAKARGGNIGDGGGAADRVINQILTEMDGMSTKKNVFIIGATNRPDIIDPAILRPGRLDQLIYIPLPDEKSRVAILKANLRKSPVAKDVDLEFLAKMTNGFSGADLTEICQRACKLAIRESIESEIRRERERQTNPSAMEVEEDDPVPEIRRDHFEEAMRFARRSVSDNDIRKYEMFAQTLQQSRGFGSFRFPSGNQGGAGPSQGSGGGTGGSVYTEDNDDDLYGDISSSSSSSSSSSSSSSSSSSSSSSSSSSSSSSSSSSSSSSSSSGGSGGSGGGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 32 - DNA, 3864 bp, synthetic construct
  source 1..3864: mol_type = other DNA
atggaaggagcaaagccgacattgcagctcgtgtaccaggcagtgcaggcgctttaccacgacccagatcccagcggaaaggagcgcgcctctttttggcttggggagctgcagcgttcggttcatgcatgggagatctcagaccagttgttacagatccggcaggatgtggagtcatgctattttgctgcacagaccatgaaaatgaagattcagacctcattttatgagctccccacagactctcatgcctctttacgggactcattgctaacccatatccagaacttgaaagacttgtcacctgttattgtaacgcagctggctttagcaatagcagatcttgccctacagatgccttcctggaagggatgtgtgcaaacactggtggaaaaatacagcaatgatgtgacttctttgccttttttgctggagatccttacagtgttacctgaagaagtacatagtcgttccttacgaattggagctaatcggcgcacagaaattatagaagatttggccttctactctagtacagtagtatctctattgatgacctgtgtagaaaaagcaggaacagatgagaaaatgcttatgaaggtttttcgctgtttgggaagttggtttaacttgggagttttggacagtaacttcatggctaacaataaattactagcactcctttttgaggttttgcaacaggataagacctcgtctaacctacatgaagctgcttcggactgtgtatgctcagctctctatgccattgagaatgtggagactaacttgccattagccatgcaactttttcagggagtgctgacattggagactgcctatcatatggccgtggcacgtgaagatttagacaaagttctgaattactgccgtattttcactgaactatgtgaaacttttcttgaaaaaattgtttgtactccaggccaaggtcttggggaccttcgaactctggagctgctgcttatctgtgcaggccatcctcaatatgaggtagtagaaatttcatttaacttttggtaccgactgggggaacatttgtacaaaactaacgatgaagttattcatggcatcttcaaagcttacattcagaggctgcttcacgccttggctcgacactgccagctggaaccagaccatgagggggttcctgaggagactgatgactttggggagtttcgcatgagggtatcagacctggtaaaggacttgattttcttgatagggtctatggagtgttttgctcagttatattctactctgaaagaaggcaacccaccctgggaggtgacagaagcggttctctttatcatggctgctatagcaaagagtgttgatccggaaaacaatccaacacttgtggaagtcctagaaggagttgtccgcctcccggagaccgtacatacggctgtgcgatacaccagcattgaattggttggagagatgagtgaagtcgttgatcgaaatcctcagttccttgaccctgtgttgggctatttgatgaaaggcctgtgtgaaaagcccctggcttctgctgcagccaaagccattcataacatttgctctgtctgccgagatcacatggctcagcactttaatggactcctggagattgcccgctccctcgattccttcctgttgtctccagaagctgctgtgggcttgctaaaagggacagcacttgtcctagcccgattacctttggataagattaccgaatgtcttagtgaactatgttctgttcaggttatggcattgaaaaagctgttgtctcaagagcccagcaatggcatatcctcagatcccacagtgttcttagatcgccttgcagtgatatttaggcataccaatcccattgtggaaaatggacagactcatccgtgtcagaaagtcatacaggaaatatggccagttttatccgagactctaaataagcaccgagctgataatcggattgtagagcgttgttgcaggtgcctgcgctttgctgttcgctgtgtaggcaaaggatctgcagcactgctgcagccactagtcacacagatggtgaatgtgtaccacgtacatcagcattcctgcttcctgtaccttggcagtatccttgtggatgaatatggcatggaagaaggctgtcggcagggactgctagacatgctccaggcactgtgcatccccacctttcagctcctagaacagcagaatggtctccagaatcaccctgacactgtagatgacctgttccggctagccaccaggtttattcagcgtagccctgtcaccttgctgcggagccaagtggtcatccctatcttacagtgggccattgcctctactaccctggaccaccgggatgccaattgtagtgtcatgaggtttctacgagacctcattcatacaggggtagccaatgatcatgaagaagactttgaattacggaaagaactgattggacaggtgatgaaccagcttggacagcagcttgtcagccagctgctgcacacctgctgcttttgcctccccccctataccctaccagatgtggctgaagtgctctgggagatcatgcaggttgacagaccgactttttgtcgatggttagaaaattccttaaaaggtttgccaaaggaaacaaccgtgggagccgtcacagtgacacacaaacaacttacagacttccacaagcaagtcactagtgctgaggaatgtaaacaagtttgctgggccttgcgagacttcaccaggttgtttcgaggtaccgagctcggatccactagtccagtgtggtggaattctgcagatatctctagttcttcaagctccagctcaagtagttccagttcctctagtagctcctcatccagctcttcttcatcatccagcagctctagcagtagttcttccagcagctctagctcctcctcatcttccggggggtctggtggcagtggagggggatccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggttga

SEQ ID NO: 33 - protein, 1287 aa, synthetic construct
MEGAKPTLQLVYQAVQALYHDPDPSGKERASFWLGELQRSVHAWEISDQLLQIRQDVESCYFAAQTMKMKIQTSFYELPTDSHASLRDSLLTHIQNLKDLSPVIVTQLALAIADLALQMPSWKGCVQTLVEKYSNDVTSLPFLLEILTVLPEEVHSRSLRIGANRRTEIIEDLAFYSSTVVSLLMTCVEKAGTDEKMLMKVFRCLGSWFNLGVLDSNFMANNKLLALLFEVLQQDKTSSNLHEAASDCVCSALYAIENVETNLPLAMQLFQGVLTLETAYHMAVAREDLDKVLNYCRIFTELCETFLEKIVCTPGQGLGDLRTLELLLICAGHPQYEVVEISFNFWYRLGEHLYKTNDEVIHGIFKAYIQRLLHALARHCQLEPDHEGVPEETDDFGEFRMRVSDLVKDLIFLIGSMECFAQLYSTLKEGNPPWEVTEAVLFIMAAIAKSVDPENNPTLVEVLEGVVRLPETVHTAVRYTSIELVGEMSEVVDRNPQFLDPVLGYLMKGLCEKPLASAAAKAIHNICSVCRDHMAQHFNGLLEIARSLDSFLLSPEAAVGLLKGTALVLARLPLDKITECLSELCSVQVMALKKLLSQEPSNGISSDPTVFLDRLAVIFRHTNPIVENGQTHPCQKVIQEIWPVLSETLNKHRADNRIVERCCRCLRFAVRCVGKGSAALLQPLVTQMVNVYHVHQHSCFLYLGSILVDEYGMEEGCRQGLLDMLQALCIPTFQLLEQQNGLQNHPDTVDDLFRLATRFIQRSPVTLLRSQVVIPILQWAIASTTLDHRDANCSVMRFLRDLIHTGVANDHEEDFELRKELIGQVMNQLGQQLVSQLLHTCCFCLPPYTLPDVAEVLWEIMQVDRPTFCRWLENSLKGLPKETTVGAVTVTHKQLTDFHKQVTSAEECKQVCWALRDFTRLFRGTELGSTSPVWWNSADISSSSSSSSSSSSSSSSSSSSSSSSSSSSSSSSSSSSSSSSSSGGSGGSGGGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 34 - DNA, 4215 bp, synthetic construct
  source 1..4215: mol_type = other DNA
atggcttctggagccgattcaaaaggtgatgacctatcaacagccattctcaaacagaagaaccgtcccaatcggttaattgttgatgaagccatcaatgaggacaacagtgtggtgtccttgtcccagcccaagatggatgaattgcagttgttccgaggtgacacagtgttgctgaaaggaaagaagagacgagaagctgtttgcatcgtcctttctgatgatacttgttctgatgagaagattcggatgaatagagttgttcggaataaccttcgtgtacgcctaggggatgtcatcagcatccagccatgccctgatgtgaagtacggcaaacgtatccatgtgctgcccattgatgacacagtggaaggcattactggtaatctcttcgaggtataccttaagccgtacttcctggaagcgtatcgacccatccggaaaggagacatttttcttgtccgtggtgggatgcgtgctgtggagttcaaagtggtggaaacagatcctagcccttattgcattgttgctccagacacagtgatccactgcgaaggggagcctatcaaacgagaggatgaggaagagtccttgaatgaagtagggtatgatgacattggtggctgcaggaagcagctagctcagataaaggagatggtggaactgcccctgagacatcctgccctctttaaggcaattggtgtgaagcctcctagaggaatcctgctttacggacctcctggaacaggaaagaccctgattgctcgagctgtagcaaatgagactggagccttcttcttcttgatcaatggtcctgagatcatgagcaaattggctggtgagtctgagagcaaccttcgtaaagcctttgaggaggctgagaagaatgctcctgccatcatcttcattgatgagctagatgccatcgctcccaaaagagagaaaactcatggcgaggtggagcggcgcattgtatcacagttgttgaccctcatggatggcctaaagcagagggcacatgtgattgttatggcagcaaccaacagacccaacagcattgacccagctctacggcgatttggtcgctttgacagggaggtagatattggaattcctgatgctacaggacgcttagagattcttcagatccataccaagaacatgaagctggcagatgatgtggacctggaacaggtagccaatgagactcacgggcatgtgggtgctgacttagcagccctgtgctcagaggctgctctgcaagccatccgcaagaagatggatctcattgacctagaggatgagaccattgatgccgaggtcatgaactctctagcagttactatggatgacttccggtgggccttgagccagagtaacccatcagcactgcgggaaaccgtggtagaggtgccacaggtaacctgggaagacatcgggggcctagaggatgtcaaacgtgagctacaggagctggtccagtatcctgtggagcacccagacaaattcctgaagtttggcatgacaccttccaagggagttctgttctatggacctcctggctgtgggaaaactttgttggccaaagccattgctaatgaatgccaggccaacttcatctccatcaagggtcctgagctgctcaccatgtggtttggggagtctgaggccaatgtcagagaaatctttgacaaggcccgccaagctgccccctgtgtgctattctttgatgagctggattcgattgccaaggctcgtggaggtaacattggagatggtggtggggctgctgaccgagtcatcaaccagatcctgacagaaatggatggcatgtccactaaaaaaaatgtgttcatcattggcgctaccaaccggcctgacatcattgatcctgccatcctcagacctggccgtcttgatcagctcatctacatcccacttcctgatgagaagtcccgtgttgccatcctcaaggctaacctgcgcaagtccccagttgccaaggatgtggacttggagttcctggctaaaatgactaatggcttctctggagctgacctgacagagatttgccagcgtgcttgcaagctggccatccgtgaatccatcgagagtgagattaggcgagaacgagagaggcagacaaacccatcagccatggaggtagaagaggatgatccagtgcctgagatccgtcgagatcactttgaagaagccatgcgctttgcgcgccgttctgtcagtgacaatgacattcggaagtatgagatgtttgcccagacccttcagcagagtcggggctttggcagcttcagattcccttcagggaaccagggtggagctggccccagtcagggcagtggaggcggcacaggtggcagtgtatacacagaagacaatgatgatgacctgtatggcgatatccaatcccgttgtttgattgcccagaccacccctgtagcagggtctcagtccctttcctctggggcagtggcaacgaccacgtcctctgctggtgatcacaatggcatgctctctgtccctgcccctggggtgccccactctgatgtgggggagccacctgcctctactggggcccagcagccttctgcattagccgccctgcagccagcaaaggagcggcggagttcctcctcgtcgtcgtcgtcctctagctcctcctcttcttcatcatcgtcgtcgtcgtcctcctcctcctctggctccagttctagtgactcagagggctctagccttcctgtgcaacctgaggtggcactgaagagggtccccagccccaccccagccccaaaggaggctgttcgagagggacgtcctccggagccaaccccagccaaacggaagaggcgctctagcagttccagttccagctcctcctcttcatcttcctcctcctcctcctcctcctcttcttcctcctcctcttcctcttcttcttcttcctcctcatcttcctcctcctcgtcgtcttcctccccttcccctgctaagcctggccctcaggccttgcccaaacctgcaagccccaagaagccaccccctggcgagcggaggtcccgcagcccccggaagccaatagactccctcagggactctcggtccctcagctactcgcctgtggagcgtcgccgtccctcgccccagccctcaccacgggaccagcagagcagcagcagtgagcggggttcccggagaggccagcgtggggacagccgctcccccagccacaagcgcaggagggagacacctagccctcggcccatgagacaccgctccggggggtctggtggcagtggagggggatccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggttga

SEQ ID NO: 35 - protein, 1404 aa, synthetic construct
MASGADSKGDDLSTAILKQKNRPNRLIVDEAINEDNSVVSLSQPKMDELQLFRGDTVLLKGKKRREAVCIVLSDDTCSDEKIRMNRVVRNNLRVRLGDVISIQPCPDVKYGKRIHVLPIDDTVEGITGNLFEVYLKPYFLEAYRPIRKGDIFLVRGGMRAVEFKVVETDPSPYCIVAPDTVIHCEGEPIKREDEEESLNEVGYDDIGGCRKQLAQIKEMVELPLRHPALFKAIGVKPPRGILLYGPPGTGKTLIARAVANETGAFFFLINGPEIMSKLAGESESNLRKAFEEAEKNAPAIIFIDELDAIAPKREKTHGEVERRIVSQLLTLMDGLKQRAHVIVMAATNRPNSIDPALRRFGRFDREVDIGIPDATGRLEILQIHTKNMKLADDVDLEQVANETHGHVGADLAALCSEAALQAIRKKMDLIDLEDETIDAEVMNSLAVTMDDFRWALSQSNPSALRETVVEVPQVTWEDIGGLEDVKRELQELVQYPVEHPDKFLKFGMTPSKGVLFYGPPGCGKTLLAKAIANECQANFISIKGPELLTMWFGESEANVREIFDKARQAAPCVLFFDELDSIAKARGGNIGDGGGAADRVINQILTEMDGMSTKKNVFIIGATNRPDIIDPAILRPGRLDQLIYIPLPDEKSRVAILKANLRKSPVAKDVDLEFLAKMTNGFSGADLTEICQRACKLAIRESIESEIRRERERQTNPSAMEVEEDDPVPEIRRDHFEEAMRFARRSVSDNDIRKYEMFAQTLQQSRGFGSFRFPSGNQGGAGPSQGSGGGTGGSVYTEDNDDDLYGDIQSRCLIAQTTPVAGSQSLSSGAVATTTSSAGDHNGMLSVPAPGVPHSDVGEPPASTGAQQPSALAALQPAKERRSSSSSSSSSSSSSSSSSSSSSSSSSGSSSSDSEGSSLPVQPEVALKRVPSPTPAPKEAVREGRPPEPTPAKRKRRSSSSSSSSSSSSSSSSSSSSSSSSSSSSSSSSSSSSSSSSSSPSPAKPGPQALPKPASPKKPPPGERRSRSPRKPIDSLRDSRSLSYSPVERRRPSPQPSPRDQQSSSSERGSRRGQRGDSRSPSHKRRRETPSPRPMRHRSGGSGGSGGGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 36 - DNA, 4611 bp, synthetic construct
  source 1..4611: mol_type = other DNA
atggaaggagcaaagccgacattgcagctcgtgtaccaggcagtgcaggcgctttaccacgacccagatcccagcggaaaggagcgcgcctctttttggcttggggagctgcagcgttcggttcatgcatgggagatctcagaccagttgttacagatccggcaggatgtggagtcatgctattttgctgcacagaccatgaaaatgaagattcagacctcattttatgagctccccacagactctcatgcctctttacgggactcattgctaacccatatccagaacttgaaagacttgtcacctgttattgtaacgcagctggctttagcaatagcagatcttgccctacagatgccttcctggaagggatgtgtgcaaacactggtggaaaaatacagcaatgatgtgacttctttgccttttttgctggagatccttacagtgttacctgaagaagtacatagtcgttccttacgaattggagctaatcggcgcacagaaattatagaagatttggccttctactctagtacagtagtatctctattgatgacctgtgtagaaaaagcaggaacagatgagaaaatgcttatgaaggtttttcgctgtttgggaagttggtttaacttgggagttttggacagtaacttcatggctaacaataaattactagcactcctttttgaggttttgcaacaggataagacctcgtctaacctacatgaagctgcttcggactgtgtatgctcagctctctatgccattgagaatgtggagactaacttgccattagccatgcaactttttcagggagtgctgacattggagactgcctatcatatggccgtggcacgtgaagatttagacaaagttctgaattactgccgtattttcactgaactatgtgaaacttttcttgaaaaaattgtttgtactccaggccaaggtcttggggaccttcgaactctggagctgctgcttatctgtgcaggccatcctcaatatgaggtagtagaaatttcatttaacttttggtaccgactgggggaacatttgtacaaaactaacgatgaagttattcatggcatcttcaaagcttacattcagaggctgcttcacgccttggctcgacactgccagctggaaccagaccatgagggggttcctgaggagactgatgactttggggagtttcgcatgagggtatcagacctggtaaaggacttgattttcttgatagggtctatggagtgttttgctcagttatattctactctgaaagaaggcaacccaccctgggaggtgacagaagcggttctctttatcatggctgctatagcaaagagtgttgatccggaaaacaatccaacacttgtggaagtcctagaaggagttgtccgcctcccggagaccgtacatacggctgtgcgatacaccagcattgaattggttggagagatgagtgaagtcgttgatcgaaatcctcagttccttgaccctgtgttgggctatttgatgaaaggcctgtgtgaaaagcccctggcttctgctgcagccaaagccattcataacatttgctctgtctgccgagatcacatggctcagcactttaatggactcctggagattgcccgctccctcgattccttcctgttgtctccagaagctgctgtgggcttgctaaaagggacagcacttgtcctagcccgattacctttggataagattaccgaatgtcttagtgaactatgttctgttcaggttatggcattgaaaaagctgttgtctcaagagcccagcaatggcatatcctcagatcccacagtgttcttagatcgccttgcagtgatatttaggcataccaatcccattgtggaaaatggacagactcatccgtgtcagaaagtcatacaggaaatatggccagttttatccgagactctaaataagcaccgagctgataatcggattgtagagcgttgttgcaggtgcctgcgctttgctgttcgctgtgtaggcaaaggatctgcagcactgctgcagccactagtcacacagatggtgaatgtgtaccacgtacatcagcattcctgcttcctgtaccttggcagtatccttgtggatgaatatggcatggaagaaggctgtcggcagggactgctagacatgctccaggcactgtgcatccccacctttcagctcctagaacagcagaatggtctccagaatcaccctgacactgtagatgacctgttccggctagccaccaggtttattcagcgtagccctgtcaccttgctgcggagccaagtggtcatccctatcttacagtgggccattgcctctactaccctggaccaccgggatgccaattgtagtgtcatgaggtttctacgagacctcattcatacaggggtagccaatgatcatgaagaagactttgaattacggaaagaactgattggacaggtgatgaaccagcttggacagcagcttgtcagccagctgctgcacacctgctgcttttgcctccccccctataccctaccagatgtggctgaagtgctctgggagatcatgcaggttgacagaccgactttttgtcgatggttagaaaattccttaaaaggtttgccaaaggaaacaaccgtgggagccgtcacagtgacacacaaacaacttacagacttccacaagcaagtcactagtgctgaggaatgtaaacaagtttgctgggccttgcgagacttcaccaggttgtttcgaggtaccgagctcggatccactagtccagtgtggtggaattctgcagatatccaatcccgttgtttgattgcccagaccacccctgtagcagggtctcagtccctttcctctggggcagtggcaacgaccacgtcctctgctggtgatcacaatggcatgctctctgtccctgcccctggggtgccccactctgatgtgggggagccacctgcctctactggggcccagcagccttctgcattagccgccctgcagccagcaaaggagcggcggagttcctcctcgtcgtcgtcgtcctctagctcctcctcttcttcatcatcgtcgtcgtcgtcctcctcctcctctggctccagttctagtgactcagagggctctagccttcctgtgcaacctgaggtggcactgaagagggtccccagccccaccccagccccaaaggaggctgttcgagagggacgtcctccggagccaaccccagccaaacggaagaggcgctctagcagttccagttccagctcctcctcttcatcttcctcctcctcctcctcctcctcttcttcctcctcctcttcctcttcttcttcttcctcctcatcttcctcctcctcgtcgtcttcctccccttcccctgctaagcctggccctcaggccttgcccaaacctgcaagccccaagaagccaccccctggcgagcggaggtcccgcagcccccggaagccaatagactccctcagggactctcggtccctcagctactcgcctgtggagcgtcgccgtccctcgccccagccctcaccacgggaccagcagagcagcagcagtgagcggggttcccggagaggccagcgtggggacagccgctcccccagccacaagcgcaggagggagacacctagccctcggcccatgagacaccgctccggggggtctggtggcagtggagggggatccgaaatcggtactggctttccattcgacccccattatgtggaagtcctgggcgagcgcatgcactacgtcgatgttggtccgcgcgatggcacccctgtgctgttcctgcacggtaacccgacctcctcctacgtgtggcgcaacatcatcccgcatgttgcaccgacccatcgctgcattgctccagacctgatcggtatgggcaaatccgacaaaccagacctgggttatttcttcgacgaccacgtccgcttcatggatgccttcatcgaagccctgggtctggaagaggtcgtcctggtcattcacgactggggctccgctctgggtttccactgggccaagcgcaatccagagcgcgtcaaaggtattgcatttatggagttcatccgccctatcccgacctgggacgaatggccagaatttgcccgcgagaccttccaggccttccgcaccaccgacgtcggccgcaagctgatcatcgatcagaacgtttttatcgagggtacgctgccgatgggtgtcgtccgcccgctgactgaagtcgagatggaccattaccgcgagccgttcctgaatcctgttgaccgcgagccactgtggcgcttcccaaacgagctgccaatcgccggtgagccagcgaacatcgtcgcgctggtcgaagaatacatggactggctgcaccagtcccctgtcccgaagctgctgttctggggcaccccaggcgttctgatcccaccggccgaagccgctcgcctggccaaaagcctgcctaactgcaaggctgtggacatcggcccgggtctgaatctgctgcaagaagacaacccggacctgatcggcagcgagatcgcgcgctggctgtctactctggagatttccggttga

SEQ ID NO: 37 - protein, 1536 aa, synthetic construct
MEGAKPTLQLVYQAVQALYHDPDPSGKERASFWLGELQRSVHAWEISDQLLQIRQDVESCYFAAQTMKMKIQTSFYELPTDSHASLRDSLLTHIQNLKDLSPVIVTQLALAIADLALQMPSWKGCVQTLVEKYSNDVTSLPFLLEILTVLPEEVHSRSLRIGANRRTEIIEDLAFYSSTVVSLLMTCVEKAGTDEKMLMKVFRCLGSWFNLGVLDSNFMANNKLLALLFEVLQQDKTSSNLHEAASDCVCSALYAIENVETNLPLAMQLFQGVLTLETAYHMAVAREDLDKVLNYCRIFTELCETFLEKIVCTPGQGLGDLRTLELLLICAGHPQYEVVEISFNFWYRLGEHLYKTNDEVIHGIFKAYIQRLLHALARHCQLEPDHEGVPEETDDFGEFRMRVSDLVKDLIFLIGSMECFAQLYSTLKEGNPPWEVTEAVLFIMAAIAKSVDPENNPTLVEVLEGVVRLPETVHTAVRYTSIELVGEMSEVVDRNPQFLDPVLGYLMKGLCEKPLASAAAKAIHNICSVCRDHMAQHFNGLLEIARSLDSFLLSPEAAVGLLKGTALVLARLPLDKITECLSELCSVQVMALKKLLSQEPSNGISSDPTVFLDRLAVIFRHTNPIVENGQTHPCQKVIQEIWPVLSETLNKHRADNRIVERCCRCLRFAVRCVGKGSAALLQPLVTQMVNVYHVHQHSCFLYLGSILVDEYGMEEGCRQGLLDMLQALCIPTFQLLEQQNGLQNHPDTVDDLFRLATRFIQRSPVTLLRSQVVIPILQWAIASTTLDHRDANCSVMRFLRDLIHTGVANDHEEDFELRKELIGQVMNQLGQQLVSQLLHTCCFCLPPYTLPDVAEVLWEIMQVDRPTFCRWLENSLKGLPKETTVGAVTVTHKQLTDFHKQVTSAEECKQVCWALRDFTRLFRGTELGSTSPVWWNSADIQSRCLIAQTTPVAGSQSLSSGAVATTTSSAGDHNGMLSVPAPGVPHSDVGEPPASTGAQQPSALAALQPAKERRSSSSSSSSSSSSSSSSSSSSSSSSSGSSSSDSEGSSLPVQPEVALKRVPSPTPAPKEAVREGRPPEPTPAKRKRRSSSSSSSSSSSSSSSSSSSSSSSSSSSSSSSSSSSSSSSSSSPSPAKPGPQALPKPASPKKPPPGERRSRSPRKPIDSLRDSRSLSYSPVERRRPSPQPSPRDQQSSSSERGSRRGQRGDSRSPSHKRRRETPSPRPMRHRSGGSGGSGGGSEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG

SEQ ID NO: 38 - protein, 305 aa, Homo sapiens
DATGRLEILQIHTKNMKLADDVDLEQVANETHGHVGADLAALCSEAALQAIRKKMDLIDLEDETIDAEVMNSLAATMDDFRWALSQSNPSALRETVVEVPQVTWEDIGGLEDVKRELQELVQYPVEHPDKFLKFGMTPSKGVLFYGPPGCGKTLLAKAIANECQANFISIKGPELLTMWFGESEANVREIFDKARQAAPCVLFFDELDSIAKARGGNIGDGGGAADRVINQILTEMDGMSTKKNVFIIGATNRPDIIDPAILRPGRPDQLIYIPLPDEKSRVAILKANLRKSPVPRMWTWSSWLK

SEQ ID NO: 39 - protein, 923 aa, Homo sapiens
MEGAKPTLQLVYQAVQALYHDPDPSGKERASFWLGELQRSVHAWEISDQLLQIRQDVESCYFAAQTMKMKIQTSFYELPTDSHASLRDSLLTHIQNLKDLSPVIVTQLALAIADLALQMPSWKGCVQTLVEKYSNDVTSLPFLLEILTVLPEEVHSRSLRIGANRRTEIIEDLAFYSSTVVSLLMTCVEKAGTDEKMLMKVFRCLGSWFNLGVLDSNFMANNKLLALLFEVLQQDKTSSNLHEAASDCVCSALYAIENVETNLPLAMQLFQGVLTLETAYHMAVAREDLDKVLNYCRIFTELCETFLEKIVCTPGQGLGDLRTLELLLICAGHPQYEVVEISFNFWYRLGEHLYKTNDEVIHGIFKAYIQRLLHALARHCQLEPDHEGVPEETDDFGEFRMRVSDLVKDLIFLIGSMECFAQLYSTLKEGNPPWEVTEAVLFIMAAIAKSVDPENNPTLVEVLEGVVRLPETVHTAVRYTSIELVGEMSEVVDRNPQFLDPVLGYLMKGLCEKPLASAAAKAIHNICSVCRDHMAQHFNGLLEIARSLDSFLLSPEAAVGLLKGTALVLARLPLDKITECLSELCSVQVMALKKLLSQEPSNGISSDPTVFLDRLAVIFRHTNPIVENGQTHPCQKVIQEIWPVLSETLNKHRADNRIVERCCRCLRFAVRCVGKGSAALLQPLVTQMVNVYHVHQHSCFLYLGSILVDEYGMEEGCRQGLLDMLQALCIPTFQLLEQQNGLQNHPDTVDDLFRLATRFIQRSPVTLLRSQVVIPILQWAIASTTLDHRDANCSVMRFLRDLIHTGVANDHEEDFELRKELIGQVMNQLGQQLVSQLLHTCCFCLPPYTLPDVAEVLWEIMQVDRPTFCRWLENSLKGLPKETTVGAVTVTHKQLTDFHKQVTSAEECKQVCWALRDFTRLFR

SEQ ID NO: 40 - protein, 294 aa, Homo sapiens
SRCLIAQTTPVAGSQSLSSGAVATTTSSAGDHNGMLSVPAPGVPHSDVGEPPASTGAQQPSALAALQPAKERRSSSSSSSSSSSSSSSSSSSSSSSSSGSSSSDSEGSSLPVQPEVALKRVPSPTPAPKEAVREGRPPEPTPAKRKRRSSSSSSSSSSSSSSSSSSSSSSSSSSSSSSSSSSSSSSSSSSPSPAKPGPQALPKPASPKKPPPGERRSRSPRKPIDSLRDSRSLSYSPVERRRPSPQPSPRDQQSSSSERGSRRGQRGDSRSPSHKRRRETPSPRPMRHRSSRSP

SEQ ID NO: 41 - RNA, 19 nt, synthetic construct
  source 1..19: mol_type = other RNA
gcatcgaatttgcagaaca

SEQ ID NO: 42 - RNA, 19 nt, synthetic construct
  source 1..19: mol_type = other RNA
cgcacacgtagagacctta

SEQ ID NO: 43 - RNA, 19 nt, synthetic construct
  source 1..19: mol_type = other RNA
ggattacctttccttgtaa

SEQ ID NO: 44 - protein, 445 aa, Homo sapiens
MAAPEERDLTQEQTEKLLQFQDLTGIESMDQCRHTLEQHNWNIEAAVQDRLNEQEGVPSVFNPPPSRPLQVNTADHRIYSYVVSRPQPRGLLGWGYYLIMLPFRFTYYTILDIFRFALRFIRPDPRSRVTDPVGDIVSFMHSFEEKYGRAHPVFYQGTYSQALNDAKRELRFLLVYLHGDDHQDSDEFCRNTLCAPEVISLINTRMLFWACSTNKPEGYRVSQALRENTYPFLAMIMLKDRRMTVVGRLEGLIQPDDLINQLTFIMDANQTYLVSERLEREERNQTQVLRQQQDEAYLASLRADQEKERKKREERERKRRKEEEVQQQKLAEERRRQNLQEEKERKLECLPPEPSPDDPESVKIIFKLPNDSRVERRFHFSQSLTVIHDFLFSLKESPEKFQIEANFPRRVLPCIPSEEWPNPPTLQEAGLSHTEVLFVQDLTDE

SEQ ID NO: 45 - protein, 216 aa, Homo sapiens
MAAPEERDLTQEQTEKLLQFQDLTGIESMDQCRHTLEQHNWNIEAAVQDRLNEQEGVPSVFNPPPSRPLQVNTADHRIYSYVVSRPQPRGLLGWGYYLIMLPFRFTYYTILDIFRFALRFIPPEPSPDDPESVKIIFKLPNDSRVERRFHFSQSLTVIHDFLFSLKESPEKFQIEANFPRRVLPCIPSEEWPNPPTLQEAGLSHTEVLFVQDLTDE

SEQ ID NO: 46 - protein, 291 aa, synthetic construct
QSRCLIAQTTPVAGSQSLSSGAVATTTSSAGDHNGMLSVPAPGVPHSDVGEPPASTGAQQPSALAALQPAKERRSSSSSSSSSSSSSSSSSSSSSSSSSGSSSSDSEGSSLPVQPEVALKRVPSPTPAPKEAVREGRPPEPTPAKRKRRSSSSSSSSSSSSSSSSSSSSSSSSSSSSSSSSSSSSSSSSSSPSPAKPGPQALPKPASPKKPPPGERRSRSPRKPIDSLRDSRSLSYSPVERRRPSPQPSPRDQQSSSSERGSRRGQRGDSRSPSHKRRRETPSPRPMRHRS

SEQ ID NO: 47 - protein, 297 aa, synthetic construct
MAEIGTGFPFDPHYVEVLGERMHYVDVGPRDGTPVLFLHGNPTSSYVWRNIIPHVAPTHRCIAPDLIGMGKSDKPDLGYFFDDHVRFMDAFIEALGLEEVVLVIHDWGSALGFHWAKRNPERVKGIAFMEFIRPIPTWDEWPEFARETFQAFRTTDVGRKLIIDQNVFIEGTLPMGVVRPLTEVEMDHYREPFLNPVDREPLWRFPNELPIAGEPANIVALVEEYMDWLHQSPVPKLLFWGTPGVLIPPAEAARLAKSLPNCKAVDIGPGLNLLQEDNPDLIGSEIARWLSTLEISG